SEQUENCE LISTING (77 sequences)

SEQ ID NO: 1 - DNA, 231 bp, Avian infectious bronchitis virus
ctagcggtacgcgggtgtgtggaagtagcgcttcagacgtatcggttctgttgcgtgaaatacggggtcacctccccccacacgcctctaagggcttttgagcctagcgttgggctacgttctcgcacaaggtcggctatacgacgtttgtagggggtagtgccaaacaacccctgaggtgacaggttctggtggtgtttagtgagcagacatacaatagacagtgacaac

SEQ ID NO: 2 - DNA, 19863 bp, Avian infectious bronchitis virus
atggctacaagcctaaaacagggagtatctcccaaaccaagggatgtcattcttgttgccaaagacattcccgaacaactctgtgacgctttgttcttctatacgtcacataaccctaaggattacgctgaagcctttgccattaggcagaagtttgaccgtaaccttcagactgggaagcagtttaaatttgaaactgtgtgtggtctcttcctcttgaagggagttgacaaaataacacctggtgtcccagcaaaagtcttaaaagccacctctaagctggctgatttggaagacatctttggtgtctctcctcttgcacgtaaatatcgtgatttgttgaagacagcatgccagtggtctctcactgtggaaacactggatgcttgtgcgcaaactcttgatgaaatctttgacccaactgaaatactttggcttcaggtagcttcaaagatccaggtttcagctatggcaatgcgcagacttgttggagaagtaactgcaagagtcatggaatctctaggttcaaatttgagcgttctttttcaagttgttaaagaacaaatagtcagaatctttgaaaaggcactggctatttttgagagtgtgaatgatttaccacagcgtattgcagcactaaaaatggcttttgccaagtgtgcgaaatcaattacagttgtggttgttggaaagactcttcttgttaaagagtttgctggaacttgtcttgcaagcattaatggtgctattgcaaaggtttttgaagaactgccaactggctttatgggtgccaaaattttcacaacacttgccttctttaaagaagcagctgtgaaaattgtggaaaatataccaaatgcaccaagaggtactagaggttttgaagtcgttggtaacgccaagggaacgcaagttgttttgcgtggcatgcgaaatgatttaactctgctcgaccaaaaagctgatatttctgttgagaaagaaggttggtctgcaattcttgaaggacatctgtgttatgtctttaagagtggtgatcgtttttatgcggcacctctttctgggaattttgcattgcatgatgtgcattgttgtgagcgtgttgtctgtctgtctgatggtgtaacaccagagataaatgatggactcattctagcagcaatctattcatcttttagtgtctcagaactcatggcagcacttaaaaagggtgaaccattcaagttcttgggtcataaatttgtgtatgcgaaggatgcagcagtctctttcactcttgcaaaagcagccactattgcagatgtactgaagctgtttcaatcagctcgtgtgcaaatagaagatgtgtggtcttcatttactgaaaagtcttttgagttctggaagatcgcatatggaaaagtgcgcaatcttgaagaatttgttaagactcatttctgtaaggctcaaatgtcaattgatattctagcagcagtgcttggcgaaggcatttggcatcttgtttcacaggtcatctataaagtatgtggtctttttactaaagtcgttgacttttgtgaaaaacactggaagggtttctgtgcacaacttaaaaaggctaagctcgttgtcacagaaactttttgtgttcttaagggagtggcacagcattgttttcaactattgctggatgcaatacattctttgtatatgagttttaagaagtgtgcacttggtagaattcatggagacttactcttctggaaagggggtgtacacaaaattgttcaagatggcgatgaagtttggtttgacgccattgatagtattgatgttgaagatctgggtgttgtcaaagaaaaacccatagattttgaggtttgtgaagacgtaacacttccagaaaaccaacctggtcacatggttcaaatcgaggatgacggaaagaactatatgttcttccgctttaaaagggatgagaacatctactacacaccaatgtctcaacttggtgcaattaatgtactttgcaaagcaggcggtaaaaccgttacctttggagagactacagtgaaagagataccgccacctgatgttgtgcctattaaggttagcatagagtgttgtggtgaaccatggaatacaattttcaagaaagcttacaaagagcccattgaagttgaaactgacctcacagtagaacaattgctctctgtgatctatgagaaaatgtgcgacgacctcaagttgtttccagaggcaccagagccaccaccatttgagaatgtcgcacttgttgataaaaacggtaaagatttggattgcataaaatcatgccaccttatttaccgtgattgtgagagcgatgatgacatcgaggaagaagatgctgaggaatgtgacacagattcagctgatgctgaggagtgtgacatcgcctcagaaagtgaagaagaggacgaggacactaaagtgctggctcttttacaagaccctgcaagtaataaataccctcttcctcttgatgatgattatagcgtctttaatggatgtattgtacataaggacgcttttgacgtcgtaaatctaccatctggtgaagaaacctttgttgtcaacaactgctttgagggagctgtaaaaccactgcctcagaaagttgttgatgttttaggtgactggggtgaggctgttgatgcgcaagagcaaattgcacaaactacttcagaggaaacccctatcagtagtttggaggcaactattgagcaagttgttgttgaggaacagaaaataatttctgttgttgaagaagaacagcaggtggcggtctacacacctgcagacctacaagttgttgaagaaacactagatgagtttattcttattgctgatgtttccacagaagaaattgtgcctcatgaagagaaggagtcacagattgaacaggagcctattcaagttgttaaatcacaacgtgaaaagaaggctaaaaagttcaaggttaaatctactacatgtgagaaacccaaatttttggagtacacaacatgtgtgggtgacctaacggtagtgattgccaaagcattggatgagtttaaagagttctgcattgtaaatgctgctaatgagcatatgtctcacggtggcggcgttgctaaggcaattgcggatttttgtggacctgattttgtggagtattgtggggactatgttaagaaacatgggcctcaacaaagacttgtcacaccttcatttgtcaaaggcattcaatgtgtgaacaatgttgtaggacctcgccatggagacagtaacttgcatgataagcttgttgctgcttacaagaatgttcttgtagatggtgttgtcaattatgttgtgccagtcctctcatcaggaatttttggtgttgattttaagatgtctatagacgctatgcgcaaggcttttgaaggttgcgacatacgcgttcttcttttctccttgtctcaagaacacatcgattatttctatgttacttgtaaacagaagacaatttatcttacagaggacggtgttaaataccgctctgctactgtgaaaccaggtgactctttgagtcaatttggaccggtttttgctagaaacaagacagtctttacagcagacgatgttgaggacaaagaaattctcttcattcctactactgacaagactgtcctcgaatattatggtttggatgcgcaaaagtatgtaatatacttgcaaactcttgcacagaagtggaatgtccaatatagggacaattttgttatacttgagtggcgtgatggaaattgctggattaatgcagcagtagtgctccttcaagctgctaagattaggtttaaaggttttcttgcagaagcatgggcacaacttttgggtggagacccaactgattttgtagcctggtgctatgcaagttgcaatgctaatgttggtgagttttcagatgctaattggcttcttgctaatttggcagaatatttcgatgctgattacacgaatgcattccttaagaggcgtgtgtcatgtaactgtggggttaagaattgtgaagttagaggccttgaagcttgtattcaaccagtaaaggcacccaatcttcttcattttaagactcagtacacaaattgtacagtgtgtgatgcaaatagtgtggatgaggtggtagaagcctcactaccatatctgttgctctttgctactgatggtcctactacagtggattgtgatgaaaatgctgtagggaatgttgttttcattggctctactaacagtggccattgttatacacaagccattggtaaggcttttgacaatcttgctaaagatagaaaatttggaaagaagtctccttacattactgcaatgtatacacacttctctcttaagggtgaaaatcctttacctgttgttaaacagagtaaaggaaaaactaaagttgtaaaaaaggatgtttctaaccttgccactagttctaaagtcagttttgataatcttactgattttgaacagtggtatgatagtaacatctacaagtgtcttaaagtacaggaaatgcctgataattttggtgaatatgtgtcatttacaacaaaggaaaattctaagttgccattgacgcttaaagttagaggtattagatcagttgttgattttaaatcaaaggatggttttacttacaagttaacacctgatactgatgaaaattcaaaggcaccagtatactaccctgtcttagatgctattagtcttaaggcaatatgggttgaaggtactgcaaattttcttgttggtcatccaaactactatagtaaatctcttcgcattcccactttttgggaaaatgcagagagctttgttaaaataggagaaaaagttgatggtgtgactactggtctttggcgtgcagaacaccttaataaacctaacttggagagaattttcaacattgctaagaaagcaattgttgggaccagtgttgttactacacaatgtggtaaattacttagtaaagcagctacattcattgctgataaactaggtgggggtgtagttcgcaatattacagatagagttaagggtctttgtggattcacacgtgggcattttgaaagaaaattgtctccacaattcataaaaacacttatattcttcttcttttattttgtaaaggctagtgctaagagtgtcgccactagttataagcgtgtgttatgtaaggtggtctttactacgctatttatattatggtttatgtacacaagtaaaccagtaatctttactggaatacgtatattagactttttatttgaaggttctgtctgtggtccttataatgactatggtaaagatacttttgatgtactacgctattgtgaagatgattttacttgtcgtgtgtgtttacatgacagggattcactacacttatataagcatgcttatagcgtagaacaattttataaaagtgcagtttctggcattagttttaattggaattggctttatttggtctttctaattttatttgttaagccagtagcgggttttgttattatttgctattgtgttaagtatttggtactgagttcaactgtattacaaactggtgtaggttttctagactggtttgttcagacagttttcactcactttaatttcatgggtgcagggttttatttctggctcttttataagatatacatacaggttcatcatatactgtattgtaaggatataacatgtgaagtgtgcaagaaggttgtacgcagcaataggcaagaggttagcgttgtagttggtggacgcaagcaaatagtgcatgtctacactaactcaggctataatttttgtaagagacataactggtattgtagaaattgtgatgagtatggtcatcaaaacacatttatgtctcccgaagttgctggcgagctctccgataagcttaagcgtcatgttaaacctacagcatatgcttaccacgttgtggatgatgcgtgcgtagttgatggttttgttaatttaaaatacaaagctgcaattcctggcaaggatagtgcatctcctgccgttaagtgttttagtgttacagatttcttgaagaaagctgtttttcttaaggaagcattgaaatgtgaacaaatatccaatgatagttttatagtgtgtaatacacagagtgcgcatgcattagaggaagcaaaaaatgctgccatctattatgcgcaatgtctgtgtaagccaatacttatacttgaccaggcactttatgagcaattagtagtggaacctgtgtctaagtgtgttgtagacaaggtgtgtagcattttgtccaatataatatctgtagatactgcagccttaaattataaagcgggcacacttcgtgattgtctgctttctgttactaaagacgaagaagctgtagatatggctatcttctgtcataaccatgatgttgaatacactactgatggttttactaatgtgataccatcttatggtgtagacactgataggttgacacctcgtgatagaggatttttaataaatgcggatgcttctgttgctaatttacgagttaaaaattctccaccagtagtatggaagttttctgatcttattaagttgtctggcagttgccttaaatatttaatttcagctactgtcaagtcagggggtcgtttctttataacaaagtctggtgctaagcaagttttttcttgccacacccagaaattgttggtagagaaaaaggcaggtggtgtcactagtgatacttttaaatggttgaaaagctgttttaagtggctatttatcttttacatactttttacagcatgttgtttgagttactactatgtggagatgaatagaagttttgttcaccccatgtatgatgtaaactctacactgcatgttgaaggttttaaagttatagataaaggtgttattagggaaattgtgcctgaagatacatgtttctctaataaatacgttaattttgatgccttctggggtaaaccatatgaaaataatagaaactgtccaattgtcactgctgttatagatggtgatgggacagtagctgctggtgttcctggttttgtatcatgggttttggatggtgctatgtttgtacatatgacacagactgagagaaaaccgtggtacattcctacttggtttaatagagaaattgtaggttacactcaggattcaattattactgagggtagtttttatacatctatagctttattttcagctagatgtttgtacctgacagccagcaatacacctcaattgtattgctttaatggtgataatgatgcacctggagccttatcattcggtagtattattcctcatagggtttacttccaaccaaatggtgttaggcttatagttcctcaacaaatatttcacacaccatacatagtaaagtttgtatcagacagctattgtagaggtagtgtatgtgaatatactaaaccaggttactgtgtatcattaaactcccaatgggttttgtttaatgatgaatacacaagtaagccaggcgtgttttgtggttctactgttagagaacttatgtttaatatggttagtacatttttcacaggtgtcaaccctaatatttatatgcagctagcaatcatgtttttgatactagttgttgttgtattaatttttgcaatggtcataaagtttcaaggtgtttttaaagcttatgcaaccgttgtgtttacaataatgttagtttgggttattaatgcatttgttttgtgtgtacatagttataatagtgttttagctgttgtattactagtactttattgttatgcctcattggtaacaagccgcaatacttctattataatgcattgttggcttgtcttcacttttggtttaatagtacccacgtggctggcttgtgtttatttagggtttattctctatatgtatacaccactatttttctggtgttatggtactactaaaaatactcgtaaactgtatgatggcaacgagtttgttggtagttatgatcttgctgcgaagagcacttttgttattcgtggttctgaatttgttaagcttacgaatgagataggtgataaatttgaagcttaccttgcagcgtatgctagacttaaatattactcaggcactggcagtgagcaagactacttgcaagcctgtcgtgcatggttagcttatgctttggaccaatatagaaatagtggtgtggaaattgtgtataccccaccacgttattctattggtgttagtagattacaggctggttttaagaaactagtttttcctagtagtgccgttgaaaggtgcattgttagtgtttcttatagaggtaataatcttaatggactgtggctaggtgatactatctactgtccgcgacatgtcctaggcaagtttacaggcgatcaatggagtgatgtacttaaccttgctaataatcatgagtttgaggttgtaactcaaaatggtgttactttgaatgttgttagtagacggttgagaggcgcagttttaattttacaaactgctgtcgccaatgctgacactcctaagtataagtttgttaaagctaattgtggagatagtttcacaattgcttgttcttatggtggtacagttgtgggactctaccctgttgctatgcgttctaatggtaccattagagcttcttttcttgcaggagcttgtggctcagttggttttaatatagaaaagggtgtagttaatttcttttatatgcaccatcttgagttacctaatgcattacacaccggaacggacctacaaggtgagttctatggtggttatgtggatgaagaggttgcacaaagagtgccaccagataatttagttactaataatattgtagcatggctttatgcagcaattattagtgttaaggagagtagtttctcactacctaaatggttggagagtactacagttactgttgatgattataataagtgggctggtgataatggttttacaccgtttgctactagtactgccattactaaattaagtgccataacgggagtagatgtttgtaaactccttcgcactattattgtaaaaagtggccaatggggtagcgaccccattttaggacaatataattttgaagatgaattgacaccagagtctgtctttaaccagattggcggtgttagattacagtcttcgtttgtaagaaaggccacatcttggttttggagtaggtgtgtgttagcttgtttcttatttgtgttgtgtgctattgtcttatttacggcagtgccacttaggtattacgtacatgcagctgttattttgttagcagctgtactttttatttcttttactgttaagcatgttatggcatatatggatactttcctactgccaacattgcttacagtcattattggagtttgtgctgaagtacctttcatctacaacactctaattagtagaatagttgtttttgttagtcaatggtatgatcccgtagtctttgatactatggtaccatggatgttactgccattagtgttgtacactgcttttaaatgtgtacaaggttgctatatgaattctttcaatacttctttgttaatgctgtatcagtttatgaagttaggttttgttatttatacctcttcgaatactcttactgcatactcagaaggtaattgggagttgttctttgatttagttcatactactgtgttggctaattgtagtagcaattctttaataggtttgcttgtgtttaagttagctaagtggatgttgtattactgcaatgcaacatactttaataattatgtgttaatggcagtcatggttaatggcataggctggctttgtacttgttactttggattgtattggtggattaataaggtttttggtttaaccttaggtaaatacaattttaaagtttcagtagaccaatataggtatatgtgtttgcataagatagacccacctaaaactgtgtgggaagttttttcgacaaatatacttatacaaggaattggtggtgatcgtgtgttgcctattgctacagttcaatctaaattgagtgatgtaaagtgtacaactgttgttttaatgcagcttttgactaagcttaatgttgaagcaaattcaaaaatgcatgcttatcttgttgatttacacaataaaatccttgcatctgatgatgttggagagtgcatggataatttgttgggtatgcttattacactattttgtatagattctactattgatttgagtgagtattgtgatgatatacttaagaggtcaactgttttacagtcagttactcaagagttctcacacataccctcttatgctgaatatgaaagagctaagaatctttatgaaaaggttttagctgattctaaaaatggtggtgtaacacagcaagagcttgctgcatatcgtaaagctgccaatattgcaaagtcagtttttgatagagacttggctgttcaaaagaagttagacagcatggcagaacgtgctatgacaacaatgtataaagaggcgcgtgtaactgatagacgagcaaaattagtttcatcactacatgcgttactgttttcaatgctcaagaaaatagattctgaaaagcttaatgtattatttgatcaggctagtagtggtgttgtacctctagctactgttccaattgtttgtagtaataagcttacccttgtaataccagatccagaaacttgggtcaagtgtgtggaaggtatgcatgttacttactcaacagttgtctggaatatagacactgttattgatgctgatggcacagaattacatcctacttctacaggtagtggattgacatactgtgtaagtggtgataatatagcgtggcctttaaaggttagcttaaatagaaatgggcataatagagttgacgttgctttgcagaataatgagcttatgcctcatggtgtaaaaacaaaggcttgcgtagcaggtgtagatcaagcacattgtagcgtagagtctaaatgttattatacaaatattagtggcaattcaattgtagctgctattacttcttcaaatccaaatctgaaagtagcttccttcttgaacgaggcaggcaaccagatttatgtagacttagacccaccttgtaagtttggtatgaaagtgggtgataaggtagaggttgtttacctgtattttataaaaaatacaaggtctattgttaggggtatggtgcttggtgctatatccaatgttgttgttttacagtctaaagggcacgagacagaggaagtggatgctgttggcatactttcactttgctcatttgcagtagatccggcagacacgtattgtaaatatgtagctgcaggcaaccaacctttaggtaactgtgttaaaatgttgacagttcataatggcagtggcttcgctataacttcaaagccaagtccaacatctgatcaagattcatatggaggagcttctgtgtgtctttattgtagagcacacatagcacatcctggcggtgcaggaaatttagatggacgttgtcaatttaaaggttcctttgtgcaaataccaactacggagagagaccctgttggattctgtctacgtaataaggtttgcactgtctgccagtgttggattggttatggatgtcagtgcgatgcgcttagacaaccaaaaccttctgttcaatcagctgctggtgcaactgattttgataagaattatttaaacgggtacggggtagcagtgaggctcggctgataccccttgctaatggatgtgaccctgatgttgtgaagcgagcctttgatgtttgtaataaggaatcagccggtatgtttcgtaatttgaagcgtaactgtgcgcggttccaagaagtacgtgatactgaagatggaaatcttgagtattgtgattcgtactttgtggttaaacaaaccactcctagtaattatgaacatgaaaagtcttgttacgaagacttaaagtctgaagtaacagctggtcatgatttctttgtgttcaataagaacatttataatattagtaggcagaggcttactaagtatactatgatggatttttgctatgctttgaggcattttgacccaaaggattgtgaagatctcaaagaaatacttgtcacttatggttgtatagaagattatcaccctaagtggtttgaagagaataaggattggtacaacccaatagaaaacccaaaatattatgctatgttggcaaaaatggggcctattgtacgtcgtgctttattgaatgctattgagttcggaaaccttatggttgaaaaaggttatgttggtgttgttacactagataaccaagatcttaatggtaaattttatgactttggtgattttcacaaaacggcacctggtgctggtgttcctgtttttgatacatattattcttacatgatgcccatcatagccatgacggatgctttggcacctgaaaggtattttgaatatgatgtgcataagggttacaagtcttatgatctcctcaagtatgattatactgaggagaaacaagagttgttccaaaaatattttaagtattgggaccaggcttaccatcctaattgccgtgactgtattgatgacaggtgtttgatacattgtgcaaacttcaacttcttgttttctacattgatacctcccacttcttttggtaatttgtgtaaattagagtttgttgatggtgtaccttttatagctacttgtggctatcattctaaagaacttggtgttattatgaatcaagataacactatgtcgttctcaaaaatgggtttaagtcaactcatgcagtttgttggagatcctgccttgttagtgggaacatccaataatttagttgatcttagaacgtcttgttttagtgtttgtgcattagcgtttggtattactcatcaaacagtaaaaccaggtcactttaacaaggatttgtatgattttgcagagaaggctggtatgtttaaagagggttcttctataccacttaaacatttcttctacccacaaactggtaatgctgctataaacgattatgattactatcgttataacaggcctaccatgtttgatatacgtcaacttctattttgtttagaagtgacttctaaatactttgattgttatgaaggcggctgtataccagcaagccaagttgtagttaacaacttagataagagcgcaggctatccatttaataagtttgggaaggcccgtctctattatgaaatgagtctagatgaacaggaccaactctctgagagtacaaagaagaatgtcttgcctactataactcaaatgaatctaaaatatgccatatccgcgaaaaatagagcgagttcagtggcaggtgtgtctatcctttctactatgactaataggcaatttcatcagaagattcttaagtctatagtcaatactagaaatgctcctgtagttattggaacaaccaaattttatggcggttgggacaatatgttgaggaacctgattcaaggtgttgaagatcctattcttatgggttgggattatcctaagtgtgatagagcaatgccaaatttgctacgtatagcagcatccttggtacttgctcgcaaacacactaattgttgtacttggtctgaacgcatttataggttgtataatgaatgcgctcaggttttatcagaaactgttttagctacaggtggtatttatgtaaaacctggtggcactagcagtggtgatgccactactgcttatgcaaacagcgttttcaacataatacaagccacatctgctaatgttgcgcgtcttttgagtgttataacgcgtgatattgtttatgatgacattaagagcttgcagtatgaattataccagcaggtttataggcgagtcaatttcgatccggcctttgttgaaaagttttattattacttatgtaagaacttttcattgatgatcttgtctgatgatggtgttgtttgttataacaatacactagccaaacaaggtcttgtagcagatatttctggttttagagagattctctattaccaaaataatgtttatatggctgattccaagtgttgggttgaacccgacttagaaaaaggcccacatgaattttgttcacaacacacaatgttagtggaggttgatggtgagcctaaatacttgccatacccagacccttcacgcattttgggtgcatgtgtctttgtagatgaagtggataagacagaacctgtggctgttatggagcgttatatagctcttgccatagacgcttacccgctagtacatcatgaaaatgaggagtacaggaaggttttctttgtgcttctttcttatattagaaaactctatcaagagctttctcagaatatgcttatggactactcttttgtaatggatatagacaagggtagtaaattttgggaacaggagttctatgaaaatatgtatagagcccctacaaccttacagtcttgtggcgtttgtgtagtgtgcaatagtcaaactatactacgctgtggtaattgcattcgcaaaccatttttgtgttgtaaatgttgttatgaccatgtcatgcacacagaccacaaaaatgttttgtctataaatccatacatctgttcgcagcctggttgcggtgaagcagatgttactaaattataccttggaggtatgtcatacttctgtggtaatcataaaccaaaattgtcaataccgttagtatctaatggtaccgtttttggaatttacagagctaattgtgcaggtagtgaaaatgttgatgattttaatcaactagctactaccaattggtctactgtggaaccctatatattggcaaatcgctgtagtgattcattgagacgctttgctgcagaaacagtaaaggctacagaggaactacacaagcaacaatttgctagtgccgaagtgagagaagtactctcagatcgtgaattaattctatcctgggagccaggtaaaactaggcctcctttgaatagaaattatgttttcacaggctaccactttacaagaactagtaaggtgcagcttggtgactttacctttgaaaaaggtgaaggtaaagatgttgtctattatagagcaacatctactgctaaattgtctgtaggagacatttttgttttaacctcacacaatgttgtttctcttgtagcgccaacgttgtgtccacagcaaaccttttctaggtttgtgaatttaagacctaatgtaatggtaccggaatgttttgtaaataacattccactttaccatttagtaggtaaacagaagcgtactacagtacaaggtcctcctggcagtggtaaatctcactttgccataggccttgctgcgtactttagtaatgctcgtgttgtctttactgcatgttcacatgcagctgttgatgctttatgtgaaaaagcatttaagtttcttaaagttgacgattgcactcgtatagtacctcaaaggactactatcgattgcttctcaaagtttaaagctaatgacacaggcaaaaagtacattttcagtactataaacgccttgccagaagttagttgtgacattcttttggttgatgaggttagtatgttgaccaattacgaattgtcttttattaatggtaagataaattaccaatatgttgtatatgtaggtgacccagctcaattaccggcacctcgtaccttacttaatggttcactttcaccaaaggattataatgttgtcacaaaccttatggtttgtgttaaaccagacatattccttgcgaagtgttaccgttgtcctaaagaaattgtagacactgtgtctactctagtatatgatggaaagtttattgcaaataacccagaatcacgtcagtgtttcaaggttatagttaataatggtaattctgatgtaggacatgaaagtggttcagcctacaacacaactcaattagagtttgtgaaagattttgtttgtcgcaataaggaatggcgggaagcaacattcatttcaccgtacaatgctatgaaccagagagcctaccgtatgcttggacttaatgttcagacagtagactcgtctcaaggttcagagtatgattatgttattttctgtgtaactgcagattcaaatcatgcactgaatattaacagatttaatgtggcgcttacaagagctaagcgtggtatactagttgtcatgcgtcagcgtgatgaattgtattctgctcttaagtttacagagctagatagtgaagcaagtctgcaaggtacaggtttgtttaaaatttgcaacaaagaatttagtggtgtccatcctgcttacgcagtcacaactaaggctcttgctgcaacttacaaggttaatgatgaacttgctgcacttgttaatgtggaagcaggttcagaaataacatataaacatcttatttctcttttaggatttaagatgagtgttagcgttgaaggttgccataatatgtttataacacgtgatgaggcaattcgcaacgtaagaggttgggtaggtttcgatgtagaagccacacatgcttgtggcactaacattggtactaacttaccttttcaagtaggtttctcgactggtgcagattttgtagtcacacctgagggacttgtagacacttcaataggcaataattttgagcctgtgaattctaaagcacctccaggcgaacaatttaatcacttgagagctttatttaagagtgctaaaccttggcatgttataagaccaaggatagtgcaaatgttagcagataacctatgcaatgtttcagattgtgtagtgtttgttacgtggtgtcatggtctagaactaactaccttgcgctattttgttaaaataggtaaggaaaaaatttgttcatgtggttctagagctacgacttttaattctcatactcaagcttacgcgtgttggaagcactgtttgggttttgactttgtctataatccgcttttagtggatattcaacaatggggttactcaggtaatctgaaatttaaccatgatttgcattgtaatgtgcatggccacgctcatgtagcttcagcggatgctattatgacccgctgccttgcaattaataatgcattttgtaaagatgtcaactgggaactgcagtatcctcatattgcaaatgaggatgaagtcaattccagttgcaggtatctacaacgcatgtatcttaatgcgtgtgttgatgctcttaaagtcaacgttgtctatgatataggcaaccctaaaggtataaaatgtgttagacgtggggatgtcaatttcaggttctacgataagaacccaattgtacctaacgtcaaacagtttgagtatgactatagtcagcataaagataagtttgctgatggtctttgtatgttttggaattgtaatgtggattgttatcctgaaaattcattagtttgtaggtatgacacacgaaatttgagtgtgtttaatttacctggttgtaatggtggtagcctgtatgttaataaacatgcattccacacacctaagttcgatcgcattagctttcgtaatttgaaagctatgccattcttcttttatgactcatcgccctgcgaaactattcaagttgatggagtagcgcaagaccttgtgtcattagcaactaaagattgtatcacaaaatgcaacattggtggtgctgtttgtaagaaacatgctcaaatgtatgcagaatttgtggcttcttataatgcagctgttacagctggttttactttttgggttactaataattttaacccttacaacttgtggaagaatttttcagcactccagtctattgacaatattgcttttaatatgtataagggtgggcattacgatgctattgcaggcgaaatacccacagtcataactggagataaagtttttgttattgatcaaggtattgaaaaggcagtttttgttaatcaaacaactctgcctacatcagttgcatttgagttgtatgcaaagagaaacattcgcacactgccaaataacagaatattgagtggtttaggtgtagacgttacccatggttttgtaatctgggattacgctaaccaaacgccactgtatcgtaatactgttaaagtatgtgcatatacagacattgaacctaatggtttaatagttctgtatgatgatagatgcggtgattatcaatcttttcttgctgctgataatgctgttttagtgtctacacagtgttataagcggtatccttatgtagaaataccatcagctctgcttgttcagaatggtatgccactaaaagatggagcgaatctgtatgtttataagaggagtagtggggcgtttgtaacgctaccaaccacattaaacacacagggtcgtaattatgaaacttttgaacctcgtagtgatgttgagagagattttcttgacatgttggaggacgattttattgaaaagtatggtaaggacctaggtctacaacacatactttatggtgaagtagaaaaaccacaattgggtggtttacacactgttataggaatgtacagactcttgcgtgctaataaattagacgcgaagtctgtaaccaattcagattctgatgtcatgcaaagctattttgttttggcagacaatggttcttacaaacaggtttgcacagttgtagaccttttgcttgatgacttcttagaacttcttagaaacatacttaaagagtatggtactaataagtccaaagttgtaacagtgtctattgattaccatagcataagttttatgacttggtttgaagaaggcagtattaaaacatgttatccacagcttcagtcggcttggacatgtggttataatatgcctgaactttataaagttcagaactgtgttatggaaccttgtaatattccaaactatggtgttggaattacattgccaagtggtattatgatgaatgtggcaaagtatacacaactttgccaatacctctctaaaactacaatgtgtgtacctcataacatgcgagtaatgcattttggagctggaagtgataaaggagttgcccctggcagcactgttcttaaacagtggcttccagaaggaacactccttgttgataacgatattgtagattatgtatctgatgcacacgtgtctgtgctgtcagattgcaataactataaaacagagcacaagtttgatcttgtgatatctgatatgtatacagataatgattcaaaaagaaaacatgaaggcatagtagccaatgatggcaatgacgatgtcttcacttatctagctaattttttaaagaacaacttagctctgggtggtagttttgccatcaaattaacagagacaagttggcatgagagtctttatgatattgcacaggattgtgcatggtggacaatgttttgtactgcagtgaatgcatcttcttcagaagcattcctgattggtgttaattatttgggtgcaagtgcaaaggttaaagttagtggaaaaaccctgcatgcaaattatatattttggaggaattgtaattatttgcaaacctctgcctatagtatatttgacgttgctaagtttgagttgaagttaaaagcaacgccagttgttaatttgaaaactgaacaaaaaacagacctagtctttaatttaataagggcaggtaaattggtcgttagagatgtcggtactattactgttaggactgactcttttgtctgctttatgtgag

SEQ ID NO: 3 - DNA, 3486 bp, Avian infectious bronchitis virus
atgtcggtactattactgttaggactgactcttttgtctgctttatgtgagtgtagtatagtaggtgacaattacacttactattaccacagcgaatatagacctcctaatggttggcatcaacaaggcggagcttatcgtgttattaatactacttcccttagtgtgtctacttgttctttaggtggtgtacctcctctactaactgtaaatgcaccatctgttactatagtagcacccactaagttgccttggtctaaagctaattttacgacagctttttgccattttggtaatttttctgtgtttgtaactcattgtactactggcaactctcggaattgtacattaacaccacctattttagcaggttatattgtagttggtgctaatggttcaattacatataattacacactaagagcgattggtgatttttcacactttcattcttttcagtgtgttagtaattttacttctgtgtacttaaatgggaaactagtttttacgtctaacctttcacttggtgttttaagtgtaggtgcatgtttaaatgcgactggcgcagagctcaagttttctgagtattttaatgtcttagcttactttataaatggtacagcacatgatgttattttatgcgatgatagccctaagggggttttggcttgccagtataatactggtaattttacagatggtttttatccaatttctgttagtactaaggctgatgatgtttttaatgtctatagaactaactctgttttaagttatggttacttacgtaacattacactaagtaattgtactgatgttaaacctaatttagggggtgaactaacactctgtcaacaatatagtgtttcggaaggttatgttaattttaatttttcttttttggatagttttacatatgtaggaccagatctttataattttgactatggttctagtcattcaaattgtcattttacacctaatactattaataatcggttgtcgtttaatactattagtgttgctataggttatggtccaatagatggtgcctgtaagcaaattagatttaggaatggttttaacccatctcatacttgctgttatgcttacagacacccatccttaagtcatagttgtcatggagttactacaggtaagtatagtaatagttatgagtgtattcttatggttgttattagtagggattatgggtctcgtgtgcagacacaaacagaggtacctactagtttaacttatttttataataatattactttggatgtgtgtgttgattataatatttatggtacattaggccaaggttatgttactaatataactgatatggccttatccggtggttacttgggttttggtggattagtcatcctagactcgtctggagctattgacatttttcttgtagaaagtgaaaatggtcctaattattataaaattaatccatgcaatgatgtcaatcagcaatatgtagtgtcagggggtagtatagttggactcttaacatctaataatttaactggttctgaattattagaagatcagttttatgttaaattcactaataatgttcataggcgtagaaggtctattagtgaaaatgttacatcttgtccatatccaacttatggaaagttttgtattaaaccagatggtagtgttagcactatagtgccagaagaagttaaagactatacttctcttttgcttaataggactgattatgtgctcataccagacagttttaatttaactgttacagatgagttcatacagacacaaatgcagcaggttcagattaattgcattcagtatgtgtgtggtaattctatacaatgcaagcaacttttccgacagtatggtcctgtatgtgatagcatactttctatagttaatggcatagtacaacaagataatgctgaaatactccagttttactcttctactaagcctaaagggtttgatgaaagctcttttagtaattttacagctggtgagtttaatatttcgcttgtattacctaagaatggacgacccacagggcgttcgtttatagaggacttgttgtttgacaaagtagaatctttaggactacctggtgactcaacttaccaaaaatgtacgtctggccccctaggttttgttaaagatctagtgtgtgcgcagaagtataatggattacttgtgttgccacctattataacggctgaaatgcaagcaatgtatactagttcattagtagcctcaatggcttttggtgggataacagctgcaggagcaataccttttgcaactcaattgcaagcacgtataaaccatcttggtattactcagacggttttgcaaaagaatcaggagaaaattgctgcatcatttaataaagctatggtgcatatgcaggatggatttaaagccacatctcttgcattacaacaagtgcaagatgttgttaacgagcatggtgcaatacttcagcagactatgcattcacttaataagaattttggtgccataacacatgtcatccaagatatatataaacaactagatgcattagaagcaaatgcacaagttgatcgtttaattaatgggagactttcgtctctatctgtattagcttcttctaaacagttagagtatacaaaagtttcgcatcaacgcgaattagctaaagaaaagattaatgagtgtgtaaaatcacaatctagcaggtatggcttctgtggtgatggcatgcatataatgtccatcccccagagtgcacctaatgggattgtgttcttgcatttcacctacacacctgtatcctttcaaaatgtgacagcagttgtgggtttttgtgttaaacctgttaatggtagtgaatacggccttgtgcctgttgttggtagaggtatttttatagaagtaaatggtacatactatatcacttctcgtgatatgtacaacccaagagctattacagggggtgatgtagttaaactggtttcgtgtcaagctaactatcaaagtattaatagaactgtcattactacatttgtagatgaagatgactttaattttgaccatgagttgtctaagtggtggaatgaaactagccgtgattttccagatctcgaccagtttaactacaccattcctgtactaaatattagtaatgagattgataggattcaagaagttatacagggacttaatgactctctaatagaccttgaaacactatcaatactcaaaacttatattaagtggccttggtatgtttggcttgccatattctttgccattattatctttatccttatactaggatgggttttcttcatgactggatgttgtggttgttgttgtgggtgctttggcattattcctctaatgagtaagtgtggtaagaaatcttcttactacactacttttgataatgatgtggtaactgaacaatacagacctaaaaagtctgtttaa

SEQ ID NO: 4 - DNA, 677 bp, Avian infectious bronchitis virus
atgattcaaagccctacatcctttctaatagtactaattcttctttggtgtaaacttgcattaagttgttttaaagagtgtgttatagcactccagcaaataatacaagttttactccaaattattaatagtaaactacagtctagacttttgctttggcacagtctagactaatgttagattttgaagcaattattgaaactggtcagcaaataattgaacaaatcagttttaatttacagcacatttcaagtgtgttaagttctgaattatttgacccctttgaagtttgtttttacagaggaggtaattattgggaggtagattcagctgacgagttttcaggtgacgatgaatatagtgaataaatcgctagaggagaacggaagtttcttaacagcagtttacatatttgttgcatttgtagcactttatctattaggtagagcgcttcaagcttttgtacaagcggctgacgcttgttgtcgtttttggtatacatgggtggtagttcctggggctaagggtacagcctttgtgtataatcatacatatggtaaaaaacttaacaaaccggatttagaaacagttattgttaacgagtttccgaaaaacggttggaaatatgggtgataacaccactaattgtactcttggtactgaacaagcagttcagc

SEQ ID NO: 5 - DNA, 681 bp, Avian infectious bronchitis virus
atgggtgataacaccactaattgtactcttggtactgaacaagcagttcagctttttaaagaatataatttatttgtaactgcattcctgttgtttctaaccatactacttcagtacggctatgcaactaggagtctgattatttacatactgaaaatgatagtgttatggtgcttctggccccttaacattgcagtaggtgtaatttcatgtatatacccaccaaacacaggaggtcttgtcgcagcgataatacttactgtgtttgcgtgtctttcttttataggttattggatccagagctttaggctctttaagcggtgcagatcatggtggtcatttaacccagaatctaacgccgtaggttcaatactcttaactaatggtcaacaatgtaattttgctatagagagtgtgcccatggtgctttctcctattataaagcatggtgttctttattgtgagggtcagtggcttgctaaatgtgaaccagaccacttgcctaaagacatatttgtatgcacaccagatagacgtaatatctatcgtatggtgcagaaatacactggtgaccaaagcggaaataagaaaaggtttgctacatttgtctatgcgaagcagtcagtagatactggcgagctagaaagtgtaacaacagctggaagtaacctttacacataa

SEQ ID NO: 6 - DNA, 359 bp, Avian infectious bronchitis virus
atgtgtgtgtgtagagagtatttaaaattgttctttgacagtgcctctattttaagagcgcggaagagtattatctttgaggatattaatataaatcctctttgtttcatactctcttttcaagagttattatttaaaaagcagtttttccactcttttgtgccaaaaactattgttgttaacggtgtaatatttcaagtggataatggaaaagtctactacgaaggcacaccagttttccaaaaaggttgttgtaggatgtggtcctattataagaaagattaaaataattaaaccacccacaacacttatttttacaaatggcgttttaggttacaaacgcttaacaaatacggacg

SEQ ID NO: 7 - DNA, 443 bp, Avian infectious bronchitis virus
atgaaatggctgactagttttggaagagctttcatctcctgttataaatccctactactaactcaattaagattattagataggttaattttagatcacggacctaagcgcacattaacgtgtgctaggcgagtgcttttatttcaattagatttagtttacaagttggcttatacacccacccaatcgctggtatgaataatagtaaaaataatccttttcgcggagcaatagcaagaaaagcgcgaatttatctgagaggaggattagattgtgtttactttcttaacaaagcaggacaagcagagccttgtcccgcgtgtacctccctagtattccaagggaaaacttgtgaggaacactattataataacaatcttttgtcatggcaagcggtaaggcaactggaaagacagacgccccagcgccagtcatcaaactag

SEQ ID NO: 8 - DNA, 1230 bp, Avian infectious bronchitis virus
atggcaagcggtaaggcaactggaaagacagacgccccagcgccagtcatcaaactaggaggaccaaagccacctaaagtcggttcttctggtaatgcatcatggtttcaaccgataaaggccaagaagctaaattcacctgtgcctaaatttgaaggtagtggtgttcctgaaaatgaaaatcttaaaacaagccatcagcacggatactggagacgccaacaccggtataaaccaggtaaaggcggaagaaaaccagttccagatgcttggtacttttattacactggaacaggaccagccgctgacctggattggggtgataaccaagatggtatagtgtgggttgctgctaaaggtgctgatactaaatctagatctaaccagggtacaagagatcctgataagtttgaccaatatccactgcgattctcagacgggggaccggatggtaacttccgttgggatttcattcctataaatcgtggtaggagtggaagatcaacagtagcttcatctgctgcttctagtagagcaccatctcgtgaagggtcacgtggacgtagaagtggagctgaagatgatcttatagctcgtgcagcaaagattattcgggaccaacaaaagaagggttctcgcattaccaaggcaaaggctgatgaaatggctcatcgccggtattgcaagcgcactgttccacccggttataaggttgatcaagtatttggtccccgtactaaaggtaaggagggaaattttggtgatgacaagatgaatgaggaaggtattaaggatgggcgtgttacagcaatgctcaacctagtccctagcagccatgcttgtctttttggaagtagagtgacgcctaaacttcaaccagatgggctccacttgaaatttgagtttactactgtggtttcacgtgatgacccgcagtttgataattatgtgaaaatttgtgatcagtgtgtcgatggtgtaggaacgcgtccacaagacgatgaaccgagaccaaagtcacgcccaaattcaagacctgcaacaagaggaaattctcctgcgccaagacaacagcgccaaaagaaggagaaaaagccaaagaagcaggaagatgatgtagatagggcattgacctcagatgaagagaggaacaatgcacagcttgagtttgatgatgaacccaaagtgattaattggggagattcagctcttggggaaaatgaattgtaa

SEQ ID NO: 9 - DNA, 418 bp, Avian infectious bronchitis virus
taacataatggacttgctgcatttgctgtcacattttgttaaatactattttgttgctttcctatcaattattacaggcattgattgtgattatgtgcaatatttaagctacttttggttgctttttgcttgttgtattgttgctgtgctttttattattgtgattctcattagtttgctttatcgtagaagtgcaatagtaagagttaaggaagataggcatgtagcttgattacctacatgtctatcgccagggaaatgtctaatctgtctacttagtagcctggaaacgaacggtagacccttagattttaatttagtttaatttttagtttagtttaagttagtttagagtaggtataaagaagccagtgccggggccacgcggagtacgatcgagggtacagcactaggacgc

SEQ ID NO: 10 - DNA, 27227 bp, Avian infectious bronchitis virus
ctagcggtacgcgggtgtgtggaagtagcgcttcagacgtatcggttctgttgcgtgaaatacggggtcacctccccccacacgcctctaagggcttttgagcctagcgttgggctacgttctcgcacaaggtcggctatacgacgtttgtagggggtagtgccaaacaacccctgaggtgacaggttctggtggtgtttagtgagcagacatacaatagacagtgacaacatggctacaagcctaaaacagggagtatctcccaaaccaagggatgtcattcttgttgccaaagacattcccgaacaactctgtgacgctttgttcttctatacgtcacataaccctaaggattacgctgaagcctttgccattaggcagaagtttgaccgtaaccttcagactgggaagcagtttaaatttgaaactgtgtgtggtctcttcctcttgaagggagttgacaaaataacacctggtgtcccagcaaaagtcttaaaagccacctctaagctggctgatttggaagacatctttggtgtctctcctcttgcacgtaaatatcgtgatttgttgaagacagcatgccagtggtctctcactgtggaaacactggatgcttgtgcgcaaactcttgatgaaatctttgacccaactgaaatactttggcttcaggtagcttcaaagatccaggtttcagctatggcaatgcgcagacttgttggagaagtaactgcaagagtcatggaatctctaggttcaaatttgagcgttctttttcaagttgttaaagaacaaatagtcagaatctttgaaaaggcactggctatttttgagagtgtgaatgatttaccacagcgtattgcagcactaaaaatggcttttgccaagtgtgcgaaatcaattacagttgtggttgttggaaagactcttcttgttaaagagtttgctggaacttgtcttgcaagcattaatggtgctattgcaaaggtttttgaagaactgccaactggctttatgggtgccaaaattttcacaacacttgccttctttaaagaagcagctgtgaaaattgtggaaaatataccaaatgcaccaagaggtactagaggttttgaagtcgttggtaacgccaagggaacgcaagttgttttgcgtggcatgcgaaatgatttaactctgctcgaccaaaaagctgatatttctgttgagaaagaaggttggtctgcaattcttgaaggacatctgtgttatgtctttaagagtggtgatcgtttttatgcggcacctctttctgggaattttgcattgcatgatgtgcattgttgtgagcgtgttgtctgtctgtctgatggtgtaacaccagagataaatgatggactcattctagcagcaatctattcatcttttagtgtctcagaactcatggcagcacttaaaaagggtgaaccattcaagttcttgggtcataaatttgtgtatgcgaaggatgcagcagtctctttcactcttgcaaaagcagccactattgcagatgtactgaagctgtttcaatcagctcgtgtgcaaatagaagatgtgtggtcttcatttactgaaaagtcttttgagttctggaagatcgcatatggaaaagtgcgcaatcttgaagaatttgttaagactcatttctgtaaggctcaaatgtcaattgatattctagcagcagtgcttggcgaaggcatttggcatcttgtttcacaggtcatctataaagtatgtggtctttttactaaagtcgttgacttttgtgaaaaacactggaagggtttctgtgcacaacttaaaaaggctaagctcgttgtcacagaaactttttgtgttcttaagggagtggcacagcattgttttcaactattgctggatgcaatacattctttgtatatgagttttaagaagtgtgcacttggtagaattcatggagacttactcttctggaaagggggtgtacacaaaattgttcaagatggcgatgaagtttggtttgacgccattgatagtattgatgttgaagatctgggtgttgtcaaagaaaaacccatagattttgaggtttgtgaagacgtaacacttccagaaaaccaacctggtcacatggttcaaatcgaggatgacggaaagaactatatgttcttccgctttaaaagggatgagaacatctactacacaccaatgtctcaacttggtgcaattaatgtactttgcaaagcaggcggtaaaaccgttacctttggagagactacagtgaaagagataccgccacctgatgttgtgcctattaaggttagcatagagtgttgtggtgaaccatggaatacaattttcaagaaagcttacaaagagcccattgaagttgaaactgacctcacagtagaacaattgctctctgtgatctatgagaaaatgtgcgacgacctcaagttgtttccagaggcaccagagccaccaccatttgagaatgtcgcacttgttgataaaaacggtaaagatttggattgcataaaatcatgccaccttatttaccgtgattgtgagagcgatgatgacatcgaggaagaagatgctgaggaatgtgacacagattcagctgatgctgaggagtgtgacatcgcctcagaaagtgaagaagaggacgaggacactaaagtgctggctcttttacaagaccctgcaagtaataaataccctcttcctcttgatgatgattatagcgtctttaatggatgtattgtacataaggacgcttttgacgtcgtaaatctaccatctggtgaagaaacctttgttgtcaacaactgctttgagggagctgtaaaaccactgcctcagaaagttgttgatgttttaggtgactggggtgaggctgttgatgcgcaagagcaaattgcacaaactacttcagaggaaacccctatcagtagtttggaggcaactattgagcaagttgttgttgaggaacagaaaataatttctgttgttgaagaagaacagcaggtggcggtctacacacctgcagacctacaagttgttgaagaaacactagatgagtttattcttattgctgatgtttccacagaagaaattgtgcctcatgaagagaaggagtcacagattgaacaggagcctattcaagttgttaaatcacaacgtgaaaagaaggctaaaaagttcaaggttaaatctactacatgtgagaaacccaaatttttggagtacacaacatgtgtgggtgacctaacggtagtgattgccaaagcattggatgagtttaaagagttctgcattgtaaatgctgctaatgagcatatgtctcacggtggcggcgttgctaaggcaattgcggatttttgtggacctgattttgtggagtattgtggggactatgttaagaaacatgggcctcaacaaagacttgtcacaccttcatttgtcaaaggcattcaatgtgtgaacaatgttgtaggacctcgccatggagacagtaacttgcatgataagcttgttgctgcttacaagaatgttcttgtagatggtgttgtcaattatgttgtgccagtcctctcatcaggaatttttggtgttgattttaagatgtctatagacgctatgcgcaaggcttttgaaggttgcgacatacgcgttcttcttttctccttgtctcaagaacacatcgattatttctatgttacttgtaaacagaagacaatttatcttacagaggacggtgttaaataccgctctgctactgtgaaaccaggtgactctttgagtcaatttggaccggtttttgctagaaacaagacagtctttacagcagacgatgttgaggacaaagaaattctcttcattcctactactgacaagactgtcctcgaatattatggtttggatgcgcaaaagtatgtaatatacttgcaaactcttgcacagaagtggaatgtccaatatagggacaattttgttatacttgagtggcgtgatggaaattgctggattaatgcagcagtagtgctccttcaagctgctaagattaggtttaaaggttttcttgcagaagcatgggcacaacttttgggtggagacccaactgattttgtagcctggtgctatgcaagttgcaatgctaatgttggtgagttttcagatgctaattggcttcttgctaatttggcagaatatttcgatgctgattacacgaatgcattccttaagaggcgtgtgtcatgtaactgtggggttaagaattgtgaagttagaggccttgaagcttgtattcaaccagtaaaggcacccaatcttcttcattttaagactcagtacacaaattgtacagtgtgtgatgcaaatagtgtggatgaggtggtagaagcctcactaccatatctgttgctctttgctactgatggtcctactacagtggattgtgatgaaaatgctgtagggaatgttgttttcattggctctactaacagtggccattgttatacacaagccattggtaaggcttttgacaatcttgctaaagatagaaaatttggaaagaagtctccttacattactgcaatgtatacacacttctctcttaagggtgaaaatcctttacctgttgttaaacagagtaaaggaaaaactaaagttgtaaaaaaggatgtttctaaccttgccactagttctaaagtcagttttgataatcttactgattttgaacagtggtatgatagtaacatctacaagtgtcttaaagtacaggaaatgcctgataattttggtgaatatgtgtcatttacaacaaaggaaaattctaagttgccattgacgcttaaagttagaggtattagatcagttgttgattttaaatcaaaggatggttttacttacaagttaacacctgatactgatgaaaattcaaaggcaccagtatactaccctgtcttagatgctattagtcttaaggcaatatgggttgaaggtactgcaaattttcttgttggtcatccaaactactatagtaaatctcttcgcattcccactttttgggaaaatgcagagagctttgttaaaataggagaaaaagttgatggtgtgactactggtctttggcgtgcagaacaccttaataaacctaacttggagagaattttcaacattgctaagaaagcaattgttgggaccagtgttgttactacacaatgtggtaaattacttagtaaagcagctacattcattgctgataaactaggtgggggtgtagttcgcaatattacagatagagttaagggtctttgtggattcacacgtgggcattttgaaagaaaattgtctccacaattcataaaaacacttatattcttcttcttttattttgtaaaggctagtgctaagagtgtcgccactagttataagcgtgtgttatgtaaggtggtctttactacgctatttatattatggtttatgtacacaagtaaaccagtaatctttactggaatacgtatattagactttttatttgaaggttctgtctgtggtccttataatgactatggtaaagatacttttgatgtactacgctattgtgaagatgattttacttgtcgtgtgtgtttacatgacagggattcactacacttatataagcatgcttatagcgtagaacaattttataaaagtgcagtttctggcattagttttaattggaattggctttatttggtctttctaattttatttgttaagccagtagcgggttttgttattatttgctattgtgttaagtatttggtactgagttcaactgtattacaaactggtgtaggttttctagactggtttgttcagacagttttcactcactttaatttcatgggtgcagggttttatttctggctcttttataagatatacatacaggttcatcatatactgtattgtaaggatataacatgtgaagtgtgcaagaaggttgtacgcagcaataggcaagaggttagcgttgtagttggtggacgcaagcaaatagtgcatgtctacactaactcaggctataatttttgtaagagacataactggtattgtagaaattgtgatgagtatggtcatcaaaacacatttatgtctcccgaagttgctggcgagctctccgataagcttaagcgtcatgttaaacctacagcatatgcttaccacgttgtggatgatgcgtgcgtagttgatggttttgttaatttaaaatacaaagctgcaattcctggcaaggatagtgcatctcctgccgttaagtgttttagtgttacagatttcttgaagaaagctgtttttcttaaggaagcattgaaatgtgaacaaatatccaatgatagttttatagtgtgtaatacacagagtgcgcatgcattagaggaagcaaaaaatgctgccatctattatgcgcaatgtctgtgtaagccaatacttatacttgaccaggcactttatgagcaattagtagtggaacctgtgtctaagtgtgttgtagacaaggtgtgtagcattttgtccaatataatatctgtagatactgcagccttaaattataaagcgggcacacttcgtgattgtctgctttctgttactaaagacgaagaagctgtagatatggctatcttctgtcataaccatgatgttgaatacactactgatggttttactaatgtgataccatcttatggtgtagacactgataggttgacacctcgtgatagaggatttttaataaatgcggatgcttctgttgctaatttacgagttaaaaattctccaccagtagtatggaagttttctgatcttattaagttgtctggcagttgccttaaatatttaatttcagctactgtcaagtcagggggtcgtttctttataacaaagtctggtgctaagcaagttttttcttgccacacccagaaattgttggtagagaaaaaggcaggtggtgtcactagtgatacttttaaatggttgaaaagctgttttaagtggctatttatcttttacatactttttacagcatgttgtttgagttactactatgtggagatgaatagaagttttgttcaccccatgtatgatgtaaactctacactgcatgttgaaggttttaaagttatagataaaggtgttattagggaaattgtgcctgaagatacatgtttctctaataaatacgttaattttgatgccttctggggtaaaccatatgaaaataatagaaactgtccaattgtcactgctgttatagatggtgatgggacagtagctgctggtgttcctggttttgtatcatgggttttggatggtgctatgtttgtacatatgacacagactgagagaaaaccgtggtacattcctacttggtttaatagagaaattgtaggttacactcaggattcaattattactgagggtagtttttatacatctatagctttattttcagctagatgtttgtacctgacagccagcaatacacctcaattgtattgctttaatggtgataatgatgcacctggagccttatcattcggtagtattattcctcatagggtttacttccaaccaaatggtgttaggcttatagttcctcaacaaatatttcacacaccatacatagtaaagtttgtatcagacagctattgtagaggtagtgtatgtgaatatactaaaccaggttactgtgtatcattaaactcccaatgggttttgtttaatgatgaatacacaagtaagccaggcgtgttttgtggttctactgttagagaacttatgtttaatatggttagtacatttttcacaggtgtcaaccctaatatttatatgcagctagcaatcatgtttttgatactagttgttgttgtattaatttttgcaatggtcataaagtttcaaggtgtttttaaagcttatgcaaccgttgtgtttacaataatgttagtttgggttattaatgcatttgttttgtgtgtacatagttataatagtgttttagctgttgtattactagtactttattgttatgcctcattggtaacaagccgcaatacttctattataatgcattgttggcttgtcttcacttttggtttaatagtacccacgtggctggcttgtgtttatttagggtttattctctatatgtatacaccactatttttctggtgttatggtactactaaaaatactcgtaaactgtatgatggcaacgagtttgttggtagttatgatcttgctgcgaagagcacttttgttattcgtggttctgaatttgttaagcttacgaatgagataggtgataaatttgaagcttaccttgcagcgtatgctagacttaaatattactcaggcactggcagtgagcaagactacttgcaagcctgtcgtgcatggttagcttatgctttggaccaatatagaaatagtggtgtggaaattgtgtataccccaccacgttattctattggtgttagtagattacaggctggttttaagaaactagtttttcctagtagtgccgttgaaaggtgcattgttagtgtttcttatagaggtaataatcttaatggactgtggctaggtgatactatctactgtccgcgacatgtcctaggcaagtttacaggcgatcaatggagtgatgtacttaaccttgctaataatcatgagtttgaggttgtaactcaaaatggtgttactttgaatgttgttagtagacggttgagaggcgcagttttaattttacaaactgctgtcgccaatgctgacactcctaagtataagtttgttaaagctaattgtggagatagtttcacaattgcttgttcttatggtggtacagttgtgggactctaccctgttgctatgcgttctaatggtaccattagagcttcttttcttgcaggagcttgtggctcagttggttttaatatagaaaagggtgtagttaatttcttttatatgcaccatcttgagttacctaatgcattacacaccggaacggacctacaaggtgagttctatggtggttatgtggatgaagaggttgcacaaagagtgccaccagataatttagttactaataatattgtagcatggctttatgcagcaattattagtgttaaggagagtagtttctcactacctaaatggttggagagtactacagttactgttgatgattataataagtgggctggtgataatggttttacaccgtttgctactagtactgccattactaaattaagtgccataacgggagtagatgtttgtaaactccttcgcactattattgtaaaaagtggccaatggggtagcgaccccattttaggacaatataattttgaagatgaattgacaccagagtctgtctttaaccagattggcggtgttagattacagtcttcgtttgtaagaaaggccacatcttggttttggagtaggtgtgtgttagcttgtttcttatttgtgttgtgtgctattgtcttatttacggcagtgccacttaggtattacgtacatgcagctgttattttgttagcagctgtactttttatttcttttactgttaagcatgttatggcatatatggatactttcctactgccaacattgcttacagtcattattggagtttgtgctgaagtacctttcatctacaacactctaattagtagaatagttgtttttgttagtcaatggtatgatcccgtagtctttgatactatggtaccatggatgttactgccattagtgttgtacactgcttttaaatgtgtacaaggttgctatatgaattctttcaatacttctttgttaatgctgtatcagtttatgaagttaggttttgttatttatacctcttcgaatactcttactgcatactcagaaggtaattgggagttgttctttgatttagttcatactactgtgttggctaattgtagtagcaattctttaataggtttgcttgtgtttaagttagctaagtggatgttgtattactgcaatgcaacatactttaataattatgtgttaatggcagtcatggttaatggcataggctggctttgtacttgttactttggattgtattggtggattaataaggtttttggtttaaccttaggtaaatacaattttaaagtttcagtagaccaatataggtatatgtgtttgcataagatagacccacctaaaactgtgtgggaagttttttcgacaaatatacttatacaaggaattggtggtgatcgtgtgttgcctattgctacagttcaatctaaattgagtgatgtaaagtgtacaactgttgttttaatgcagcttttgactaagcttaatgttgaagcaaattcaaaaatgcatgcttatcttgttgatttacacaataaaatccttgcatctgatgatgttggagagtgcatggataatttgttgggtatgcttattacactattttgtatagattctactattgatttgagtgagtattgtgatgatatacttaagaggtcaactgttttacagtcagttactcaagagttctcacacataccctcttatgctgaatatgaaagagctaagaatctttatgaaaaggttttagctgattctaaaaatggtggtgtaacacagcaagagcttgctgcatatcgtaaagctgccaatattgcaaagtcagtttttgatagagacttggctgttcaaaagaagttagacagcatggcagaacgtgctatgacaacaatgtataaagaggcgcgtgtaactgatagacgagcaaaattagtttcatcactacatgcgttactgttttcaatgctcaagaaaatagattctgaaaagcttaatgtattatttgatcaggctagtagtggtgttgtacctctagctactgttccaattgtttgtagtaataagcttacccttgtaataccagatccagaaacttgggtcaagtgtgtggaaggtatgcatgttacttactcaacagttgtctggaatatagacactgttattgatgctgatggcacagaattacatcctacttctacaggtagtggattgacatactgtgtaagtggtgataatatagcgtggcctttaaaggttagcttaaatagaaatgggcataatagagttgacgttgctttgcagaataatgagcttatgcctcatggtgtaaaaacaaaggcttgcgtagcaggtgtagatcaagcacattgtagcgtagagtctaaatgttattatacaaatattagtggcaattcaattgtagctgctattacttcttcaaatccaaatctgaaagtagcttccttcttgaacgaggcaggcaaccagatttatgtagacttagacccaccttgtaagtttggtatgaaagtgggtgataaggtagaggttgtttacctgtattttataaaaaatacaaggtctattgttaggggtatggtgcttggtgctatatccaatgttgttgttttacagtctaaagggcacgagacagaggaagtggatgctgttggcatactttcactttgctcatttgcagtagatccggcagacacgtattgtaaatatgtagctgcaggcaaccaacctttaggtaactgtgttaaaatgttgacagttcataatggcagtggcttcgctataacttcaaagccaagtccaacatctgatcaagattcatatggaggagcttctgtgtgtctttattgtagagcacacatagcacatcctggcggtgcaggaaatttagatggacgttgtcaatttaaaggttcctttgtgcaaataccaactacggagagagaccctgttggattctgtctacgtaataaggtttgcactgtctgccagtgttggattggttatggatgtcagtgcgatgcgcttagacaaccaaaaccttctgttcaatcagctgctggtgcaactgattttgataagaattatttaaacgggtacggggtagcagtgaggctcggctgataccccttgctaatggatgtgaccctgatgttgtgaagcgagcctttgatgtttgtaataaggaatcagccggtatgtttcgtaatttgaagcgtaactgtgcgcggttccaagaagtacgtgatactgaagatggaaatcttgagtattgtgattcgtactttgtggttaaacaaaccactcctagtaattatgaacatgaaaagtcttgttacgaagacttaaagtctgaagtaacagctggtcatgatttctttgtgttcaataagaacatttataatattagtaggcagaggcttactaagtatactatgatggatttttgctatgctttgaggcattttgacccaaaggattgtgaagatctcaaagaaatacttgtcacttatggttgtatagaagattatcaccctaagtggtttgaagagaataaggattggtacaacccaatagaaaacccaaaatattatgctatgttggcaaaaatggggcctattgtacgtcgtgctttattgaatgctattgagttcggaaaccttatggttgaaaaaggttatgttggtgttgttacactagataaccaagatcttaatggtaaattttatgactttggtgattttcacaaaacggcacctggtgctggtgttcctgtttttgatacatattattcttacatgatgcccatcatagccatgacggatgctttggcacctgaaaggtattttgaatatgatgtgcataagggttacaagtcttatgatctcctcaagtatgattatactgaggagaaacaagagttgttccaaaaatattttaagtattgggaccaggcttaccatcctaattgccgtgactgtattgatgacaggtgtttgatacattgtgcaaacttcaacttcttgttttctacattgatacctcccacttcttttggtaatttgtgtaaattagagtttgttgatggtgtaccttttatagctacttgtggctatcattctaaagaacttggtgttattatgaatcaagataacactatgtcgttctcaaaaatgggtttaagtcaactcatgcagtttgttggagatcctgccttgttagtgggaacatccaataatttagttgatcttagaacgtcttgttttagtgtttgtgcattagcgtttggtattactcatcaaacagtaaaaccaggtcactttaacaaggatttgtatgattttgcagagaaggctggtatgtttaaagagggttcttctataccacttaaacatttcttctacccacaaactggtaatgctgctataaacgattatgattactatcgttataacaggcctaccatgtttgatatacgtcaacttctattttgtttagaagtgacttctaaatactttgattgttatgaaggcggctgtataccagcaagccaagttgtagttaacaacttagataagagcgcaggctatccatttaataagtttgggaaggcccgtctctattatgaaatgagtctagatgaacaggaccaactctctgagagtacaaagaagaatgtcttgcctactataactcaaatgaatctaaaatatgccatatccgcgaaaaatagagcgagttcagtggcaggtgtgtctatcctttctactatgactaataggcaatttcatcagaagattcttaagtctatagtcaatactagaaatgctcctgtagttattggaacaaccaaattttatggcggttgggacaatatgttgaggaacctgattcaaggtgttgaagatcctattcttatgggttgggattatcctaagtgtgatagagcaatgccaaatttgctacgtatagcagcatccttggtacttgctcgcaaacacactaattgttgtacttggtctgaacgcatttataggttgtataatgaatgcgctcaggttttatcagaaactgttttagctacaggtggtatttatgtaaaacctggtggcactagcagtggtgatgccactactgcttatgcaaacagcgttttcaacataatacaagccacatctgctaatgttgcgcgtcttttgagtgttataacgcgtgatattgtttatgatgacattaagagcttgcagtatgaattataccagcaggtttataggcgagtcaatttcgatccggcctttgttgaaaagttttattattacttatgtaagaacttttcattgatgatcttgtctgatgatggtgttgtttgttataacaatacactagccaaacaaggtcttgtagcagatatttctggttttagagagattctctattaccaaaataatgtttatatggctgattccaagtgttgggttgaacccgacttagaaaaaggcccacatgaattttgttcacaacacacaatgttagtggaggttgatggtgagcctaaatacttgccatacccagacccttcacgcattttgggtgcatgtgtctttgtagatgaagtggataagacagaacctgtggctgttatggagcgttatatagctcttgccatagacgcttacccgctagtacatcatgaaaatgaggagtacaggaaggttttctttgtgcttctttcttatattagaaaactctatcaagagctttctcagaatatgcttatggactactcttttgtaatggatatagacaagggtagtaaattttgggaacaggagttctatgaaaatatgtatagagcccctacaaccttacagtcttgtggcgtttgtgtagtgtgcaatagtcaaactatactacgctgtggtaattgcattcgcaaaccatttttgtgttgtaaatgttgttatgaccatgtcatgcacacagaccacaaaaatgttttgtctataaatccatacatctgttcgcagcctggttgcggtgaagcagatgttactaaattataccttggaggtatgtcatacttctgtggtaatcataaaccaaaattgtcaataccgttagtatctaatggtaccgtttttggaatttacagagctaattgtgcaggtagtgaaaatgttgatgattttaatcaactagctactaccaattggtctactgtggaaccctatatattggcaaatcgctgtagtgattcattgagacgctttgctgcagaaacagtaaaggctacagaggaactacacaagcaacaatttgctagtgccgaagtgagagaagtactctcagatcgtgaattaattctatcctgggagccaggtaaaactaggcctcctttgaatagaaattatgttttcacaggctaccactttacaagaactagtaaggtgcagcttggtgactttacctttgaaaaaggtgaaggtaaagatgttgtctattatagagcaacatctactgctaaattgtctgtaggagacatttttgttttaacctcacacaatgttgtttctcttgtagcgccaacgttgtgtccacagcaaaccttttctaggtttgtgaatttaagacctaatgtaatggtaccggaatgttttgtaaataacattccactttaccatttagtaggtaaacagaagcgtactacagtacaaggtcctcctggcagtggtaaatctcactttgccataggccttgctgcgtactttagtaatgctcgtgttgtctttactgcatgttcacatgcagctgttgatgctttatgtgaaaaagcatttaagtttcttaaagttgacgattgcactcgtatagtacctcaaaggactactatcgattgcttctcaaagtttaaagctaatgacacaggcaaaaagtacattttcagtactataaacgccttgccagaagttagttgtgacattcttttggttgatgaggttagtatgttgaccaattacgaattgtcttttattaatggtaagataaattaccaatatgttgtatatgtaggtgacccagctcaattaccggcacctcgtaccttacttaatggttcactttcaccaaaggattataatgttgtcacaaaccttatggtttgtgttaaaccagacatattccttgcgaagtgttaccgttgtcctaaagaaattgtagacactgtgtctactctagtatatgatggaaagtttattgcaaataacccagaatcacgtcagtgtttcaaggttatagttaataatggtaattctgatgtaggacatgaaagtggttcagcctacaacacaactcaattagagtttgtgaaagattttgtttgtcgcaataaggaatggcgggaagcaacattcatttcaccgtacaatgctatgaaccagagagcctaccgtatgcttggacttaatgttcagacagtagactcgtctcaaggttcagagtatgattatgttattttctgtgtaactgcagattcaaatcatgcactgaatattaacagatttaatgtggcgcttacaagagctaagcgtggtatactagttgtcatgcgtcagcgtgatgaattgtattctgctcttaagtttacagagctagatagtgaagcaagtctgcaaggtacaggtttgtttaaaatttgcaacaaagaatttagtggtgtccatcctgcttacgcagtcacaactaaggctcttgctgcaacttacaaggttaatgatgaacttgctgcacttgttaatgtggaagcaggttcagaaataacatataaacatcttatttctcttttaggatttaagatgagtgttagcgttgaaggttgccataatatgtttataacacgtgatgaggcaattcgcaacgtaagaggttgggtaggtttcgatgtagaagccacacatgcttgtggcactaacattggtactaacttaccttttcaagtaggtttctcgactggtgcagattttgtagtcacacctgagggacttgtagacacttcaataggcaataattttgagcctgtgaattctaaagcacctccaggcgaacaatttaatcacttgagagctttatttaagagtgctaaaccttggcatgttataagaccaaggatagtgcaaatgttagcagataacctatgcaatgtttcagattgtgtagtgtttgttacgtggtgtcatggtctagaactaactaccttgcgctattttgttaaaataggtaaggaaaaaatttgttcatgtggttctagagctacgacttttaattctcatactcaagcttacgcgtgttggaagcactgtttgggttttgactttgtctataatccgcttttagtggatattcaacaatggggttactcaggtaatctgaaatttaaccatgatttgcattgtaatgtgcatggccacgctcatgtagcttcagcggatgctattatgacccgctgccttgcaattaataatgcattttgtaaagatgtcaactgggaactgcagtatcctcatattgcaaatgaggatgaagtcaattccagttgcaggtatctacaacgcatgtatcttaatgcgtgtgttgatgctcttaaagtcaacgttgtctatgatataggcaaccctaaaggtataaaatgtgttagacgtggggatgtcaatttcaggttctacgataagaacccaattgtacctaacgtcaaacagtttgagtatgactatagtcagcataaagataagtttgctgatggtctttgtatgttttggaattgtaatgtggattgttatcctgaaaattcattagtttgtaggtatgacacacgaaatttgagtgtgtttaatttacctggttgtaatggtggtagcctgtatgttaataaacatgcattccacacacctaagttcgatcgcattagctttcgtaatttgaaagctatgccattcttcttttatgactcatcgccctgcgaaactattcaagttgatggagtagcgcaagaccttgtgtcattagcaactaaagattgtatcacaaaatgcaacattggtggtgctgtttgtaagaaacatgctcaaatgtatgcagaatttgtggcttcttataatgcagctgttacagctggttttactttttgggttactaataattttaacccttacaacttgtggaagaatttttcagcactccagtctattgacaatattgcttttaatatgtataagggtgggcattacgatgctattgcaggcgaaatacccacagtcataactggagataaagtttttgttattgatcaaggtattgaaaaggcagtttttgttaatcaaacaactctgcctacatcagttgcatttgagttgtatgcaaagagaaacattcgcacactgccaaataacagaatattgagtggtttaggtgtagacgttacccatggttttgtaatctgggattacgctaaccaaacgccactgtatcgtaatactgttaaagtatgtgcatatacagacattgaacctaatggtttaatagttctgtatgatgatagatgcggtgattatcaatcttttcttgctgctgataatgctgttttagtgtctacacagtgttataagcggtatccttatgtagaaataccatcagctctgcttgttcagaatggtatgccactaaaagatggagcgaatctgtatgtttataagaggagtagtggggcgtttgtaacgctaccaaccacattaaacacacagggtcgtaattatgaaacttttgaacctcgtagtgatgttgagagagattttcttgacatgttggaggacgattttattgaaaagtatggtaaggacctaggtctacaacacatactttatggtgaagtagaaaaaccacaattgggtggtttacacactgttataggaatgtacagactcttgcgtgctaataaattagacgcgaagtctgtaaccaattcagattctgatgtcatgcaaagctattttgttttggcagacaatggttcttacaaacaggtttgcacagttgtagaccttttgcttgatgacttcttagaacttcttagaaacatacttaaagagtatggtactaataagtccaaagttgtaacagtgtctattgattaccatagcataagttttatgacttggtttgaagaaggcagtattaaaacatgttatccacagcttcagtcggcttggacatgtggttataatatgcctgaactttataaagttcagaactgtgttatggaaccttgtaatattccaaactatggtgttggaattacattgccaagtggtattatgatgaatgtggcaaagtatacacaactttgccaatacctctctaaaactacaatgtgtgtacctcataacatgcgagtaatgcattttggagctggaagtgataaaggagttgcccctggcagcactgttcttaaacagtggcttccagaaggaacactccttgttgataacgatattgtagattatgtatctgatgcacacgtgtctgtgctgtcagattgcaataactataaaacagagcacaagtttgatcttgtgatatctgatatgtatacagataatgattcaaaaagaaaacatgaaggcatagtagccaatgatggcaatgacgatgtcttcacttatctagctaattttttaaagaacaacttagctctgggtggtagttttgccatcaaattaacagagacaagttggcatgagagtctttatgatattgcacaggattgtgcatggtggacaatgttttgtactgcagtgaatgcatcttcttcagaagcattcctgattggtgttaattatttgggtgcaagtgcaaaggttaaagttagtggaaaaaccctgcatgcaaattatatattttggaggaattgtaattatttgcaaacctctgcctatagtatatttgacgttgctaagtttgagttgaagttaaaagcaacgccagttgttaatttgaaaactgaacaaaaaacagacctagtctttaatttaataagggcaggtaaattggtcgttagagatgtcggtactattactgttaggactgactcttttgtctgctttatgtgagtgtagtatagtaggtgacaattacacttactattaccacagcgaatatagacctcctaatggttggcatcaacaaggcggagcttatcgtgttattaatactacttcccttagtgtgtctacttgttctttaggtggtgtacctcctctactaactgtaaatgcaccatctgttactatagtagcacccactaagttgccttggtctaaagctaattttacgacagctttttgccattttggtaatttttctgtgtttgtaactcattgtactactggcaactctcggaattgtacattaacaccacctattttagcaggttatattgtagttggtgctaatggttcaattacatataattacacactaagagcgattggtgatttttcacactttcattcttttcagtgtgttagtaattttacttctgtgtacttaaatgggaaactagtttttacgtctaacctttcacttggtgttttaagtgtaggtgcatgtttaaatgcgactggcgcagagctcaagttttctgagtattttaatgtcttagcttactttataaatggtacagcacatgatgttattttatgcgatgatagccctaagggggttttggcttgccagtataatactggtaattttacagatggtttttatccaatttctgttagtactaaggctgatgatgtttttaatgtctatagaactaactctgttttaagttatggttacttacgtaacattacactaagtaattgtactgatgttaaacctaatttagggggtgaactaacactctgtcaacaatatagtgtttcggaaggttatgttaattttaatttttcttttttggatagttttacatatgtaggaccagatctttataattttgactatggttctagtcattcaaattgtcattttacacctaatactattaataatcggttgtcgtttaatactattagtgttgctataggttatggtccaatagatggtgcctgtaagcaaattagatttaggaatggttttaacccatctcatacttgctgttatgcttacagacacccatccttaagtcatagttgtcatggagttactacaggtaagtatagtaatagttatgagtgtattcttatggttgttattagtagggattatgggtctcgtgtgcagacacaaacagaggtacctactagtttaacttatttttataataatattactttggatgtgtgtgttgattataatatttatggtacattaggccaaggttatgttactaatataactgatatggccttatccggtggttacttgggttttggtggattagtcatcctagactcgtctggagctattgacatttttcttgtagaaagtgaaaatggtcctaattattataaaattaatccatgcaatgatgtcaatcagcaatatgtagtgtcagggggtagtatagttggactcttaacatctaataatttaactggttctgaattattagaagatcagttttatgttaaattcactaataatgttcataggcgtagaaggtctattagtgaaaatgttacatcttgtccatatccaacttatggaaagttttgtattaaaccagatggtagtgttagcactatagtgccagaagaagttaaagactatacttctcttttgcttaataggactgattatgtgctcataccagacagttttaatttaactgttacagatgagttcatacagacacaaatgcagcaggttcagattaattgcattcagtatgtgtgtggtaattctatacaatgcaagcaacttttccgacagtatggtcctgtatgtgatagcatactttctatagttaatggcatagtacaacaagataatgctgaaatactccagttttactcttctactaagcctaaagggtttgatgaaagctcttttagtaattttacagctggtgagtttaatatttcgcttgtattacctaagaatggacgacccacagggcgttcgtttatagaggacttgttgtttgacaaagtagaatctttaggactacctggtgactcaacttaccaaaaatgtacgtctggccccctaggttttgttaaagatctagtgtgtgcgcagaagtataatggattacttgtgttgccacctattataacggctgaaatgcaagcaatgtatactagttcattagtagcctcaatggcttttggtgggataacagctgcaggagcaataccttttgcaactcaattgcaagcacgtataaaccatcttggtattactcagacggttttgcaaaagaatcaggagaaaattgctgcatcatttaataaagctatggtgcatatgcaggatggatttaaagccacatctcttgcattacaacaagtgcaagatgttgttaacgagcatggtgcaatacttcagcagactatgcattcacttaataagaattttggtgccataacacatgtcatccaagatatatataaacaactagatgcattagaagcaaatgcacaagttgatcgtttaattaatgggagactttcgtctctatctgtattagcttcttctaaacagttagagtatacaaaagtttcgcatcaacgcgaattagctaaagaaaagattaatgagtgtgtaaaatcacaatctagcaggtatggcttctgtggtgatggcatgcatataatgtccatcccccagagtgcacctaatgggattgtgttcttgcatttcacctacacacctgtatcctttcaaaatgtgacagcagttgtgggtttttgtgttaaacctgttaatggtagtgaatacggccttgtgcctgttgttggtagaggtatttttatagaagtaaatggtacatactatatcacttctcgtgatatgtacaacccaagagctattacagggggtgatgtagttaaactggtttcgtgtcaagctaactatcaaagtattaatagaactgtcattactacatttgtagatgaagatgactttaattttgaccatgagttgtctaagtggtggaatgaaactagccgtgattttccagatctcgaccagtttaactacaccattcctgtactaaatattagtaatgagattgataggattcaagaagttatacagggacttaatgactctctaatagaccttgaaacactatcaatactcaaaacttatattaagtggccttggtatgtttggcttgccatattctttgccattattatctttatccttatactaggatgggttttcttcatgactggatgttgtggttgttgttgtgggtgctttggcattattcctctaatgagtaagtgtggtaagaaatcttcttactacactacttttgataatgatgtggtaactgaacaatacagacctaaaaagtctgtttaatgattcaaagccctacatcctttctaatagtactaattcttctttggtgtaaacttgcattaagttgttttaaagagtgtgttatagcactccagcaaataatacaagttttactccaaattattaatagtaaactacagtctagacttttgctttggcacagtctagactaatgttagattttgaagcaattattgaaactggtcagcaaataattgaacaaatcagttttaatttacagcacatttcaagtgtgttaagttctgaattatttgacccctttgaagtttgtttttacagaggaggtaattattgggaggtagattcagctgacgagttttcaggtgacgatgaatatagtgaataaatcgctagaggagaacggaagtttcttaacagcagtttacatatttgttgcatttgtagcactttatctattaggtagagcgcttcaagcttttgtacaagcggctgacgcttgttgtcgtttttggtatacatgggtggtagttcctggggctaagggtacagcctttgtgtataatcatacatatggtaaaaaacttaacaaaccggatttagaaacagttattgttaacgagtttccgaaaaacggttggaaatatgggtgataacaccactaattgtactcttggtactgaacaagcagttcagctttttaaagaatataatttatttgtaactgcattcctgttgtttctaaccatactacttcagtacggctatgcaactaggagtctgattatttacatactgaaaatgatagtgttatggtgcttctggccccttaacattgcagtaggtgtaatttcatgtatatacccaccaaacacaggaggtcttgtcgcagcgataatacttactgtgtttgcgtgtctttcttttataggttattggatccagagctttaggctctttaagcggtgcagatcatggtggtcatttaacccagaatctaacgccgtaggttcaatactcttaactaatggtcaacaatgtaattttgctatagagagtgtgcccatggtgctttctcctattataaagcatggtgttctttattgtgagggtcagtggcttgctaaatgtgaaccagaccacttgcctaaagacatatttgtatgcacaccagatagacgtaatatctatcgtatggtgcagaaatacactggtgaccaaagcggaaataagaaaaggtttgctacatttgtctatgcgaagcagtcagtagatactggcgagctagaaagtgtaacaacagctggaagtaacctttacacataaatgtgtgtgtgtagagagtatttaaaattgttctttgacagtgcctctattttaagagcgcggaagagtattatctttgaggatattaatataaatcctctttgtttcatactctcttttcaagagttattatttaaaaagcagtttttccactcttttgtgccaaaaactattgttgttaacggtgtaatatttcaagtggataatggaaaagtctactacgaaggcacaccagttttccaaaaaggttgttgtaggatgtggtcctattataagaaagattaaaataattaaaccacccacaacacttatttttacaaatggcgttttaggttacaaacgcttaacaaatacggacgatgaaatggctgactagttttggaagagctttcatctcctgttataaatccctactactaactcaattaagattattagataggttaattttagatcacggacctaagcgcacattaacgtgtgctaggcgagtgcttttatttcaattagatttagtttacaagttggcttatacacccacccaatcgctggtatgaataatagtaaaaataatccttttcgcggagcaatagcaagaaaagcgcgaatttatctgagaggaggattagattgtgtttactttcttaacaaagcaggacaagcagagccttgtcccgcgtgtacctccctagtattccaagggaaaacttgtgaggaacactattataataacaatcttttgtcatggcaagcggtaaggcaactggaaagacagacgccccagcgccagtcatcaaactaggaggaccaaagccacctaaagtcggttcttctggtaatgcatcatggtttcaaccgataaaggccaagaagctaaattcacctgtgcctaaatttgaaggtagtggtgttcctgaaaatgaaaatcttaaaacaagccatcagcacggatactggagacgccaacaccggtataaaccaggtaaaggcggaagaaaaccagttccagatgcttggtacttttattacactggaacaggaccagccgctgacctggattggggtgataaccaagatggtatagtgtgggttgctgctaaaggtgctgatactaaatctagatctaaccagggtacaagagatcctgataagtttgaccaatatccactgcgattctcagacgggggaccggatggtaacttccgttgggatttcattcctataaatcgtggtaggagtggaagatcaacagtagcttcatctgctgcttctagtagagcaccatctcgtgaagggtcacgtggacgtagaagtggagctgaagatgatcttatagctcgtgcagcaaagattattcgggaccaacaaaagaagggttctcgcattaccaaggcaaaggctgatgaaatggctcatcgccggtattgcaagcgcactgttccacccggttataaggttgatcaagtatttggtccccgtactaaaggtaaggagggaaattttggtgatgacaagatgaatgaggaaggtattaaggatgggcgtgttacagcaatgctcaacctagtccctagcagccatgcttgtctttttggaagtagagtgacgcctaaacttcaaccagatgggctccacttgaaatttgagtttactactgtggtttcacgtgatgacccgcagtttgataattatgtgaaaatttgtgatcagtgtgtcgatggtgtaggaacgcgtccacaagacgatgaaccgagaccaaagtcacgcccaaattcaagacctgcaacaagaggaaattctcctgcgccaagacaacagcgccaaaagaaggagaaaaagccaaagaagcaggaagatgatgtagatagggcattgacctcagatgaagagaggaacaatgcacagcttgagtttgatgatgaacccaaagtgattaattggggagattcagctcttggggaaaatgaattgtaagtaacataatggacttgctgcatttgctgtcacattttgttaaatactattttgttgctttcctatcaattattacaggcattgattgtgattatgtgcaatatttaagctacttttggttgctttttgcttgttgtattgttgctgtgctttttattattgtgattctcattagtttgctttatcgtagaagtgcaatagtaagagttaaggaagataggcatgtagcttgattacctacatgtctatcgccagggaaatgtctaatctgtctacttagtagcctggaaacgaacggtagacccttagattttaatttagtttaatttttagtttagtttaagttagtttagagtaggtataaagaagccagtgccggggccacgcggagtacgatcgagggtacagcactaggacgc

SEQ ID NO: 11 - protein, 3942 aa, Avian infectious bronchitis virus
MATSLKQGVSPKPRDVILVAKDIPEQLCDALFFYTSHNPKDYAEAFAIRQKFDRNLQTGKQFKFETVCGLFLLKGVDKITPGVPAKVLKATSKLADLEDIFGVSPLARKYRDLLKTACQWSLTVETLDACAQTLDEIFDPTEILWLQVASKIQVSAMAMRRLVGEVTARVMESLGSNLSVLFQVVKEQIVRIFEKALAIFESVNDLPQRIAALKMAFAKCAKSITVVVVGKTLLVKEFAGTCLASINGAIAKVFEELPTGFMGAKIFTTLAFFKEAAVKIVENIPNAPRGTRGFEVVGNAKGTQVVLRGMRNDLTLLDQKADISVEKEGWSAILEGHLCYVFKSGDRFYAAPLSGNFALHDVHCCERVVCLSDGVTPEINDGLILAAIYSSFSVSELMAALKKGEPFKFLGHKFVYAKDAAVSFTLAKAATIADVLKLFQSARVQIEDVWSSFTEKSFEFWKIAYGKVRNLEEFVKTHFCKAQMSIDILAAVLGEGIWHLVSQVIYKVCGLFTKVVDFCEKHWKGFCAQLKKAKLVVTETFCVLKGVAQHCFQLLLDAIHSLYMSFKKCALGRIHGDLLFWKGGVHKIVQDGDEVWFDAIDSIDVEDLGVVKEKPIDFEVCEDVTLPENQPGHMVQIEDDGKNYMFFRFKRDENIYYTPMSQLGAINVLCKAGGKTVTFGETTVKEIPPPDVVPIKVSIECCGEPWNTIFKKAYKEPIEVETDLTVEQLLSVIYEKMCDDLKLFPEAPEPPPFENVALVDKNGKDLDCIKSCHLIYRDCESDDDIEEEDAEECDTDSADAEECDIASESEEEDEDTKVLALLQDPASNKYPLPLDDDYSVFNGCIVHKDAFDVVNLPSGEETFVVNNCFEGAVKPLPQKVVDVLGDWGEAVDAQEQIAQTTSEETPISSLEATIEQVVVEEQKIISVVEEEQQVAVYTPADLQVVEETLDEFILIADVSTEEIVPHEEKESQIEQEPIQVVKSQREKKAKKFKVKSTTCEKPKFLEYTTCVGDLTVVIAKALDEFKEFCIVNAANEHMSHGGGVAKAIADFCGPDFVEYCGDYVKKHGPQQRLVTPSFVKGIQCVNNVVGPRHGDSNLHDKLVAAYKNVLVDGVVNYVVPVLSSGIFGVDFKMSIDAMRKAFEGCDIRVLLFSLSQEHIDYFYVTCKQKTIYLTEDGVKYRSATVKPGDSLSQFGPVFARNKTVFTADDVEDKEILFIPTTDKTVLEYYGLDAQKYVIYLQTLAQKWNVQYRDNFVILEWRDGNCWINAAVVLLQAAKIRFKGFLAEAWAQLLGGDPTDFVAWCYASCNANVGEFSDANWLLANLAEYFDADYTNAFLKRRVSCNCGVKNCEVRGLEACIQPVKAPNLLHFKTQYTNCTVCDANSVDEVVEASLPYLLLFATDGPTTVDCDENAVGNVVFIGSTNSGHCYTQAIGKAFDNLAKDRKFGKKSPYITAMYTHFSLKGENPLPVVKQSKGKTKVVKKDVSNLATSSKVSFDNLTDFEQWYDSNIYKCLKVQEMPDNFGEYVSFTTKENSKLPLTLKVRGIRSVVDFKSKDGFTYKLTPDTDENSKAPVYYPVLDAISLKAIWVEGTANFLVGHPNYYSKSLRIPTFWENAESFVKIGEKVDGVTTGLWRAEHLNKPNLERIFNIAKKAIVGTSVVTTQCGKLLSKAATFIADKLGGGVVRNITDRVKGLCGFTRGHFERKLSPQFIKTLIFFFFYFVKASAKSVATSYKRVLCKVVFTTLFILWFMYTSKPVIFTGIRILDFLFEGSVCGPYNDYGKDTFDVLRYCEDDFTCRVCLHDRDSLHLYKHAYSVEQFYKSAVSGISFNWNWLYLVFLILFVKPVAGFVIICYCVKYLVLSSTVLQTGVGFLDWFVQTVFTHFNFMGAGFYFWLFYKIYIQVHHILYCKDITCEVCKKVVRSNRQEVSVVVGGRKQIVHVYTNSGYNFCKRHNWYCRNCDEYGHQNTFMSPEVAGELSDKLKRHVKPTAYAYHVVDDACVVDGFVNLKYKAAIPGKDSASPAVKCFSVTDFLKKAVFLKEALKCEQISNDSFIVCNTQSAHALEEAKNAAIYYAQCLCKPILILDQALYEQLVVEPVSKCVVDKVCSILSNIISVDTAALNYKAGTLRDCLLSVTKDEEAVDMAIFCHNHDVEYTTDGFTNVIPSYGVDTDRLTPRDRGFLINADASVANLRVKNSPPVVWKFSDLIKLSGSCLKYLISATVKSGGRFFITKSGAKQVFSCHTQKLLVEKKAGGVTSDTFKWLKSCFKWLFIFYILFTACCLSYYYVEMNRSFVHPMYDVNSTLHVEGFKVIDKGVIREIVPEDTCFSNKYVNFDAFWGKPYENNRNCPIVTAVIDGDGTVAAGVPGFVSWVLDGAMFVHMTQTERKPWYIPTWFNREIVGYTQDSIITEGSFYTSIALFSARCLYLTASNTPQLYCFNGDNDAPGALSFGSIIPHRVYFQPNGVRLIVPQQIFHTPYIVKFVSDSYCRGSVCEYTKPGYCVSLNSQWVLFNDEYTSKPGVFCGSTVRELMFNMVSTFFTGVNPNIYMQLAIMFLILVVVVLIFAMVIKFQGVFKAYATVVFTIMLVWVINAFVLCVHSYNSVLAVVLLVLYCYASLVTSRNTSIIMHCWLVFTFGLIVPTWLACVYLGFILYMYTPLFFWCYGTTKNTRKLYDGNEFVGSYDLAAKSTFVIRGSEFVKLTNEIGDKFEAYLAAYARLKYYSGTGSEQDYLQACRAWLAYALDQYRNSGVEIVYTPPRYSIGVSRLQAGFKKLVFPSSAVERCIVSVSYRGNNLNGLWLGDTIYCPRHVLGKFTGDQWSDVLNLANNHEFEVVTQNGVTLNVVSRRLRGAVLILQTAVANADTPKYKFVKANCGDSFTIACSYGGTVVGLYPVAMRSNGTIRASFLAGACGSVGFNIEKGVVNFFYMHHLELPNALHTGTDLQGEFYGGYVDEEVAQRVPPDNLVTNNIVAWLYAAIISVKESSFSLPKWLESTTVTVDDYNKWAGDNGFTPFATSTAITKLSAITGVDVCKLLRTIIVKSGQWGSDPILGQYNFEDELTPESVFNQIGGVRLQSSFVRKATSWFWSRCVLACFLFVLCAIVLFTAVPLRYYVHAAVILLAAVLFISFTVKHVMAYMDTFLLPTLLTVIIGVCAEVPFIYNTLISRIVVFVSQWYDPVVFDTMVPWMLLPLVLYTAFKCVQGCYMNSFNTSLLMLYQFMKLGFVIYTSSNTLTAYSEGNWELFFDLVHTTVLANCSSNSLIGLLVFKLAKWMLYYCNATYFNNYVLMAVMVNGIGWLCTCYFGLYWWINKVFGLTLGKYNFKVSVDQYRYMCLHKIDPPKTVWEVFSTNILIQGIGGDRVLPIATVQSKLSDVKCTTVVLMQLLTKLNVEANSKMHAYLVDLHNKILASDDVGECMDNLLGMLITLFCIDSTIDLSEYCDDILKRSTVLQSVTQEFSHIPSYAEYERAKNLYEKVLADSKNGGVTQQELAAYRKAANIAKSVFDRDLAVQKKLDSMAERAMTTMYKEARVTDRRAKLVSSLHALLFSMLKKIDSEKLNVLFDQASSGVVPLATVPIVCSNKLTLVIPDPETWVKCVEGMHVTYSTVVWNIDTVIDADGTELHPTSTGSGLTYCVSGDNIAWPLKVSLNRNGHNRVDVALQNNELMPHGVKTKACVAGVDQAHCSVESKCYYTNISGNSIVAAITSSNPNLKVASFLNEAGNQIYVDLDPPCKFGMKVGDKVEVVYLYFIKNTRSIVRGMVLGAISNVVVLQSKGHETEEVDAVGILSLCSFAVDPADTYCKYVAAGNQPLGNCVKMLTVHNGSGFAITSKPSPTSDQDSYGGASVCLYCRAHIAHPGGAGNLDGRCQFKGSFVQIPTTERDPVGFCLRNKVCTVCQCWIGYGCQCDALRQPKPSVQSAAGATDFDKNYLNGYGVAVRLG

SEQ ID NO: 12 - protein, 2652 aa, Avian infectious bronchitis virus
MFRNLKRNCARFQEVRDTEDGNLEYCDSYFVVKQTTPSNYEHEKSCYEDLKSEVTAGHDFFVFNKNIYNISRQRLTKYTMMDFCYALRHFDPKDCEDLKEILVTYGCIEDYHPKWFEENKDWYNPIENPKYYAMLAKMGPIVRRALLNAIEFGNLMVEKGYVGVVTLDNQDLNGKFYDFGDFHKTAPGAGVPVFDTYYSYMMPIIAMTDALAPERYFEYDVHKGYKSYDLLKYDYTEEKQELFQKYFKYWDQAYHPNCRDCIDDRCLIHCANFNFLFSTLIPPTSFGNLCKLEFVDGVPFIATCGYHSKELGVIMNQDNTMSFSKMGLSQLMQFVGDPALLVGTSNNLVDLRTSCFSVCALAFGITHQTVKPGHFNKDLYDFAEKAGMFKEGSSIPLKHFFYPQTGNAAINDYDYYRYNRPTMFDIRQLLFCLEVTSKYFDCYEGGCIPASQVVVNNLDKSAGYPFNKFGKARLYYEMSLDEQDQLSESTKKNVLPTITQMNLKYAISAKNRASSVAGVSILSTMTNRQFHQKILKSIVNTRNAPVVIGTTKFYGGWDNMLRNLIQGVEDPILMGWDYPKCDRAMPNLLRIAASLVLARKHTNCCTWSERIYRLYNECAQVLSETVLATGGIYVKPGGTSSGDATTAYANSVFNIIQATSANVARLLSVITRDIVYDDIKSLQYELYQQVYRRVNFDPAFVEKFYYYLCKNFSLMILSDDGVVCYNNTLAKQGLVADISGFREILYYQNNVYMADSKCWVEPDLEKGPHEFCSQHTMLVEVDGEPKYLPYPDPSRILGACVFVDEVDKTEPVAVMERYIALAIDAYPLVHHENEEYRKVFFVLLSYIRKLYQELSQNMLMDYSFVMDIDKGSKFWEQEFYENMYRAPTTLQSCGVCVVCNSQTILRCGNCIRKPFLCCKCCYDHVMHTDHKNVLSINPYICSQPGCGEADVTKLYLGGMSYFCGNHKPKLSIPLVSNGTVFGIYRANCAGSENVDDFNQLATTNWSTVEPYILANRCSDSLRRFAAETVKATEELHKQQFASAEVREVLSDRELILSWEPGKTRPPLNRNYVFTGYHFTRTSKVQLGDFTFEKGEGKDVVYYRATSTAKLSVGDIFVLTSHNVVSLVAPTLCPQQTFSRFVNLRPNVMVPECFVNNIPLYHLVGKQKRTTVQGPPGSGKSHFAIGLAAYFSNARVVFTACSHAAVDALCEKAFKFLKVDDCTRIVPQRTTIDCFSKFKANDTGKKYIFSTINALPEVSCDILLVDEVSMLTNYELSFINGKINYQYVVYVGDPAQLPAPRTLLNGSLSPKDYNVVTNLMVCVKPDIFLAKCYRCPKEIVDTVSTLVYDGKFIANNPESRQCFKVIVNNGNSDVGHESGSAYNTTQLEFVKDFVCRNKEWREATFISPYNAMNQRAYRMLGLNVQTVDSSQGSEYDYVIFCVTADSNHALNINRFNVALTRAKRGILVVMRQRDELYSALKFTELDSEASLQGTGLFKICNKEFSGVHPAYAVTTKALAATYKVNDELAALVNVEAGSEITYKHLISLLGFKMSVSVEGCHNMFITRDEAIRNVRGWVGFDVEATHACGTNIGTNLPFQVGFSTGADFVVTPEGLVDTSIGNNFEPVNSKAPPGEQFNHLRALFKSAKPWHVIRPRIVQMLADNLCNVSDCVVFVTWCHGLELTTLRYFVKIGKEKICSCGSRATTFNSHTQAYACWKHCLGFDFVYNPLLVDIQQWGYSGNLKFNHDLHCNVHGHAHVASADAIMTRCLAINNAFCKDVNWELQYPHIANEDEVNSSCRYLQRMYLNACVDALKVNVVYDIGNPKGIKCVRRGDVNFRFYDKNPIVPNVKQFEYDYSQHKDKFADGLCMFWNCNVDCYPENSLVCRYDTRNLSVFNLPGCNGGSLYVNKHAFHTPKFDRISFRNLKAMPFFFYDSSPCETIQVDGVAQDLVSLATKDCITKCNIGGAVCKKHAQMYAEFVASYNAAVTAGFTFWVTNNFNPYNLWKNFSALQSIDNIAFNMYKGGHYDAIAGEIPTVITGDKVFVIDQGIEKAVFVNQTTLPTSVAFELYAKRNIRTLPNNRILSGLGVDVTHGFVIWDYANQTPLYRNTVKVCAYTDIEPNGLIVLYDDRCGDYQSFLAADNAVLVSTQCYKRYPYVEIPSALLVQNGMPLKDGANLYVYKRSSGAFVTLPTTLNTQGRNYETFEPRSDVERDFLDMLEDDFIEKYGKDLGLQHILYGEVEKPQLGGLHTVIGMYRLLRANKLDAKSVTNSDSDVMQSYFVLADNGSYKQVCTVVDLLLDDFLELLRNILKEYGTNKSKVVTVSIDYHSISFMTWFEEGSIKTCYPQLQSAWTCGYNMPELYKVQNCVMEPCNIPNYGVGITLPSGIMMNVAKYTQLCQYLSKTTMCVPHNMRVMHFGAGSDKGVAPGSTVLKQWLPEGTLLVDNDIVDYVSDAHVSVLSDCNNYKTEHKFDLVISDMYTDNDSKRKHEGIVANDGNDDVFTYLANFLKNNLALGGSFAIKLTETSWHESLYDIAQDCAWWTMFCTAVNASSSEAFLIGVNYLGASAKVKVSGKTLHANYIFWRNCNYLQTSAYSIFDVAKFELKLKATPVVNLKTEQKTDLVFNLIRAGKLVVRDVGTITVRTDSFVCFM

SEQ ID NO: 13 - protein, 1161 aa, Avian infectious bronchitis virus
MSVLLLLGLTLLSALCECSIVGDNYTYYYHSEYRPPNGWHQQGGAYRVINTTSLSVSTCSLGGVPPLLTVNAPSVTIVAPTKLPWSKANFTTAFCHFGNFSVFVTHCTTGNSRNCTLTPPILAGYIVVGANGSITYNYTLRAIGDFSHFHSFQCVSNFTSVYLNGKLVFTSNLSLGVLSVGACLNATGAELKFSEYFNVLAYFINGTAHDVILCDDSPKGVLACQYNTGNFTDGFYPISVSTKADDVFNVYRTNSVLSYGYLRNITLSNCTDVKPNLGGELTLCQQYSVSEGYVNFNFSFLDSFTYVGPDLYNFDYGSSHSNCHFTPNTINNRLSFNTISVAIGYGPIDGACKQIRFRNGFNPSHTCCYAYRHPSLSHSCHGVTTGKYSNSYECILMVVISRDYGSRVQTQTEVPTSLTYFYNNITLDVCVDYNIYGTLGQGYVTNITDMALSGGYLGFGGLVILDSSGAIDIFLVESENGPNYYKINPCNDVNQQYVVSGGSIVGLLTSNNLTGSELLEDQFYVKFTNNVHRRRRSISENVTSCPYPTYGKFCIKPDGSVSTIVPEEVKDYTSLLLNRTDYVLIPDSFNLTVTDEFIQTQMQQVQINCIQYVCGNSIQCKQLFRQYGPVCDSILSIVNGIVQQDNAEILQFYSSTKPKGFDESSFSNFTAGEFNISLVLPKNGRPTGRSFIEDLLFDKVESLGLPGDSTYQKCTSGPLGFVKDLVCAQKYNGLLVLPPIITAEMQAMYTSSLVASMAFGGITAAGAIPFATQLQARINHLGITQTVLQKNQEKIAASFNKAMVHMQDGFKATSLALQQVQDVVNEHGAILQQTMHSLNKNFGAITHVIQDIYKQLDALEANAQVDRLINGRLSSLSVLASSKQLEYTKVSHQRELAKEKINECVKSQSSRYGFCGDGMHIMSIPQSAPNGIVFLHFTYTPVSFQNVTAVVGFCVKPVNGSEYGLVPVVGRGIFIEVNGTYYITSRDMYNPRAITGGDVVKLVSCQANYQSINRTVITTFVDEDDFNFDHELSKWWNETSRDFPDLDQFNYTIPVLNISNEIDRIQEVIQGLNDSLIDLETLSILKTYIKWPWYVWLAIFFAIIIFILILGWVFFMTGCCGCCCGCFGIIPLMSKCGKKSSYYTTFDNDVVTEQYRPKKSV

SEQ ID NO: 14 - protein, 57 aa, Avian infectious bronchitis virus
MIQSPTSFLIVLILLWCKLALSCFKECVIALQQIIQVLLQIINSKLQSRLLLWHSLD

SEQ ID NO: 15 - protein, 64 aa, Avian infectious bronchitis virus
MLDFEAIIETGQQIIEQISFNLQHISSVLSSELFDPFEVCFYRGGNYWEVDSADEFSGDDEYSE

SEQ ID NO: 16 - protein, 93 aa, Avian infectious bronchitis virus
MNIVNKSLEENGSFLTAVYIFVAFVALYLLGRALQAFVQAADACCRFWYTWVVVPGAKGTAFVYNHTYGKKLNKPDLETVIVNEFPKNGWKYG

SEQ ID NO: 17 - protein, 226 aa, Avian infectious bronchitis virus
MGDNTTNCTLGTEQAVQLFKEYNLFVTAFLLFLTILLQYGYATRSLIIYILKMIVLWCFWPLNIAVGVISCIYPPNTGGLVAAIILTVFACLSFIGYWIQSFRLFKRCRSWWSFNPESNAVGSILLTNGQQCNFAIESVPMVLSPIIKHGVLYCEGQWLAKCEPDHLPKDIFVCTPDRRNIYRMVQKYTGDQSGNKKRFATFVYAKQSVDTGELESVTTAGSNLYT

SEQ ID NO: 18 - protein, 65 aa, Avian infectious bronchitis virus
MKWLTSFGRAFISCYKSLLLTQLRLLDRLILDHGPKRTLTCARRVLLFQLDLVYKLAYTPTQSLV

SEQ ID NO: 19 - protein, 82 aa, Avian infectious bronchitis virus
MNNSKNNPFRGAIARKARIYLRGGLDCVYFLNKAGQAEPCPACTSLVFQGKTCEEHYYNNNLLSWQAVRQLERQTPQRQSSN

SEQ ID NO: 20 - protein, 409 aa, Avian infectious bronchitis virus
MASGKATGKTDAPAPVIKLGGPKPPKVGSSGNASWFQPIKAKKLNSPVPKFEGSGVPENENLKTSHQHGYWRRQHRYKPGKGGRKPVPDAWYFYYTGTGPAADLDWGDNQDGIVWVAAKGADTKSRSNQGTRDPDKFDQYPLRFSDGGPDGNFRWDFIPINRGRSGRSTVASSAASSRAPSREGSRGRRSGAEDDLIARAAKIIRDQQKKGSRITKAKADEMAHRRYCKRTVPPGYKVDQVFGPRTKGKEGNFGDDKMNEEGIKDGRVTAMLNLVPSSHACLFGSRVTPKLQPDGLHLKFEFTTVVSRDDPQFDNYVKICDQCVDGVGTRPQDDEPRPKSRPNSRPATRGNSPAPRQQRQKKEKKPKKQEDDVDRALTSDEERNNAQLEFDDEPKVINWGDSALGENEL

SEQ ID NO: 21 - DNA, 18 bp, Artificial Sequence
  IBV-specific primer for reverse transcription
tttagtaaaaagaccacc

SEQ ID NO: 22 - DNA, 16 bp, Artificial Sequence
  IBV-specific primer for reverse transcription
catacttttgcgcatc

SEQ ID NO: 23 - DNA, 16 bp, Artificial Sequence
  IBV-specific primer for reverse transcription
agaaaacctacaccag

SEQ ID NO: 24 - DNA, 18 bp, Artificial Sequence
  IBV-specific primer for reverse transcription
gtaaagaatgtactaacc

SEQ ID NO: 25 - DNA, 20 bp, Artificial Sequence
  IBV-specific primer for reverse transcription
atagtatcaaagactacagg

SEQ ID NO: 26 - DNA, 16 bp, Artificial Sequence
  IBV-specific primer for reverse transcription
ctccataagaatcctg

SEQ ID NO: 27 - DNA, 17 bp, Artificial Sequence
  IBV-specific primer for reverse transcription
taaaacttggttgttcc

SEQ ID NO: 28 - DNA, 18 bp, Artificial Sequence
  IBV-specific primer for reverse transcription
ttcacataaagcatcaac

SEQ ID NO: 29 - DNA, 16 bp, Artificial Sequence
  IBV-specific primer for reverse transcription
gtcatactcaaactgc

SEQ ID NO: 30 - DNA, 17 bp, Artificial Sequence
  IBV-specific primer for reverse transcription
caaaatgcattactcgc

SEQ ID NO: 31 - DNA, 18 bp, Artificial Sequence
  IBV-specific primer for reverse transcription
catatcttctttttgacc

SEQ ID NO: 32 - DNA, 19 bp, Artificial Sequence
  IBV-specific primer for reverse transcription
tttgaatcattaaacagac

SEQ ID NO: 33 - DNA, 16 bp, Artificial Sequence
  IBV-specific primer for reverse transcription
accaactttaggtggc

SEQ ID NO: 34 - DNA, 17 bp, Artificial Sequence
  IBV-specific primer for reverse transcription
ttgctctaactctatac

SEQ ID NO: 35 - DNA, 27 bp, Artificial Sequence
  Primer for genome amplification
gcgctagatttccaacttaacaaaacg

SEQ ID NO: 36 - DNA, 27 bp, Artificial Sequence
  Primer for genome amplification
gacttgcgaaacaagatgccaaatgcc

SEQ ID NO: 37 - DNA, 26 bp, Artificial Sequence
  Primer for genome amplification
tggaggcttgcatatggaaaagtgcg

SEQ ID NO: 38 - DNA, 35 bp, Artificial Sequence
  Primer for genome amplification
ggaatgaagagaatttctttatcctcaacatcatc

SEQ ID NO: 39 - DNA, 23 bp, Artificial Sequence
  Primer for genome amplification
cggaggatggtgttaaataccgc

SEQ ID NO: 40 - DNA, 36 bp, Artificial Sequence
  Primer for genome amplification
caaataatattagaaagaccaaataaagccaattcc

SEQ ID NO: 41 - DNA, 31 bp, Artificial Sequence
  Primer for genome amplification
gattcttttgatgtgttacgctattgtgcag

SEQ ID NO: 42 - DNA, 29 bp, Artificial Sequence
  Primer for genome amplification
cctggtttagtatactcacatacactacc

SEQ ID NO: 43 - DNA, 26 bp, Artificial Sequence
  Primer for genome amplification
cctaatggtgttaggcttatagttcc

SEQ ID NO: 44 - DNA, 29 bp, Artificial Sequence
  Primer for genome amplification
gtatcaaagactacaggatcataccattg

SEQ ID NO: 45 - DNA, 27 bp, Artificial Sequence
  Primer for genome amplification
cagttattattggagtttgtgctgaag

SEQ ID NO: 46 - DNA, 27 bp, Artificial Sequence
  Primer for genome amplification
gaatcctgatccggagttggacttggc

SEQ ID NO: 47 - DNA, 26 bp, Artificial Sequence
  Primer for genome amplification
gtggcagcaggtaatcaacctttagg

SEQ ID NO: 48 - DNA, 30 bp, Artificial Sequence
  Primer for genome amplification
ataaaacttggttgttccaataactacagg

SEQ ID NO: 49 - DNA, 31 bp, Artificial Sequence
  Primer for genome amplification
gtgtctatcctttctactatgactaataggc

SEQ ID NO: 50 - DNA, 27 bp, Artificial Sequence
  Primer for genome amplification
cacataaagcatcaacagctgcatgag

SEQ ID NO: 51 - DNA, 25 bp, Artificial Sequence
  Primer for genome amplification
ggcaagcagaagcgtactacagtac

SEQ ID NO: 52 - DNA, 28 bp, Artificial Sequence
  Primer for genome amplification
ctgcttgacattgggtactattggattc

SEQ ID NO: 53 - DNA, 30 bp, Artificial Sequence
  Primer for genome amplification
cgttgtctatgatataggcaaccctaaagg

SEQ ID NO: 54 - DNA, 28 bp, Artificial Sequence
  Primer for genome amplification
gtattgacagagttgtgtatactttgcc

SEQ ID NO: 55 - DNA, 27 bp, Artificial Sequence
  Primer for genome amplification
gtaacagtgtcaattgattaccatagc

SEQ ID NO: 56 - DNA, 28 bp, Artificial Sequence
  Primer for genome amplification
tccatacgcgtttgtatgtactcatctg

SEQ ID NO: 57 - DNA, 25 bp, Artificial Sequence
  Primer for genome amplification
cataggcgtagaaggtctattagtg

SEQ ID NO: 58 - DNA, 28 bp, Artificial Sequence
  Primer for genome amplification
ttatttgctggagtgctataacacactc

SEQ ID NO: 59 - DNA, 27 bp, Artificial Sequence
  Primer for genome amplification
cattatgcctctaatgagtaagtgtgg

SEQ ID NO: 60 - DNA, 24 bp, Artificial Sequence
  Primer for genome amplification
aactttaggtggctttggtcctcc

SEQ ID NO: 61 - DNA, 28 bp, Artificial Sequence
  Primer for genome amplification
gaaaagcgcgaatttatctgagagaagg

SEQ ID NO: 62 - DNA, 39 bp, Artificial Sequence
  Primer for genome amplification
catagccaattaaacttaacttaaactaaaatttagctc

SEQ ID NO: 63 - DNA, 26 bp, Artificial Sequence
  Sequencing primer located in the middle of the PCR product
cctaaggattacgctgaagcctttgc

SEQ ID NO: 64 - DNA, 24 bp, Artificial Sequence
  Sequencing primer located in the middle of the PCR product
cacaccaatgtctcaacttggtgc

SEQ ID NO: 65 - DNA, 26 bp, Artificial Sequence
  Sequencing primer located in the middle of the PCR product
tccaatgctttggcaatcactaccgt

SEQ ID NO: 66 - DNA, 25 bp, Artificial Sequence
  Sequencing primer located in the middle of the PCR product
gtagaagcctcactaccatatctgt

SEQ ID NO: 67 - DNA, 25 bp, Artificial Sequence
  Sequencing primer located in the middle of the PCR product
gttaaacctacagcatatgcttacc

SEQ ID NO: 68 - DNA, 24 bp, Artificial Sequence
  Sequencing primer located in the middle of the PCR product
gtatgatggcaacgagtttgttgg

SEQ ID NO: 69 - DNA, 26 bp, Artificial Sequence
  Sequencing primer located in the middle of the PCR product
tccttgcatctgatgatgttggagag

SEQ ID NO: 70 - DNA, 25 bp, Artificial Sequence
  Sequencing primer located in the middle of the PCR product
tgacccaaaggattgtgaagatctc

SEQ ID NO: 71 - DNA, 26 bp, Artificial Sequence
  Sequencing primer located in the middle of the PCR product
caaggtcttgtagcagatatttctgg

SEQ ID NO: 72 - DNA, 24 bp, Artificial Sequence
  Sequencing primer located in the middle of the PCR product
catgaaagtggttcagcctacaac

SEQ ID NO: 73 - DNA, 26 bp, Artificial Sequence
  Sequencing primer located in the middle of the PCR product
tgcaagtgcaaaggttaaagttagtg

SEQ ID NO: 74 - DNA, 26 bp, Artificial Sequence
  Sequencing primer located in the middle of the PCR product
gtaagcataacagcaagtatgagatg

SEQ ID NO: 75 - DNA, 26 bp, Artificial Sequence
  Sequencing primer located in the middle of the PCR product
ctagttcattagtagcctcaatggct

SEQ ID NO: 76 - DNA, 25 bp, Artificial Sequence
  Sequencing primer located in the middle of the PCR product
cttggtactgaacaagcagttcagc

SEQ ID NO: 77 - DNA, 25 bp, Artificial Sequence
  Sequencing primer located in the middle of the PCR product
tcgtgcagcaaagattattcgggac

(19) United States
(12) Patent Application Publication
(10) Pub. No.: US 2023/0190909 A1
(43) Pub. Date: Jun. 22, 2023

(54) INFECTIOUS BRONCHITIS (IB) VIRUS VARIANTS AND RELATED COMPOSITIONS, USES AND METHOD

(71) Applicant: ANICON LABOR GMBH, Höltinghausen (DE)

(72) Inventors: Martin LIMAN, Hatten (DE); Theresa MENKE, Herßum (DE); Jennifer HANEKE, Cloppenburg (DE); Wiebke BIELENBERG, Syke (DE); Natalie ß VOGEL, Diepholz (DE); Swaantje RÖNCHEN, Vechta (DE); Diana PETZOLDT, Cloppenburg (DE); Klaus-Peter BEHR, Großenkneten (DE)

(21) Appl. No.: 16/324,744

(22) PCT Filed: Aug. 14, 2017
(86) PCT No.: PCT/EP2017/070616

§ 371 Date: Feb. 11, 2019

(30) Foreign Application Priority Data
Aug. 12, 2016 (DE) 10 2016 215 118.5

(51) Int. Cl. A61K 39/12; A61P 31/14; C07K 16/10; C12N 7/00

(52) U.S. Cl. CPC A61K 39/12; A61P 31/14; C07K 16/10; C12N 7/00; A61K 2039/5254

(57) ABSTRACT

The invention relates to agents, compositions, uses and methods of a new variant of the infectious bronchitis-virus (IB-virus).

The invention relates to agents, compositions, uses and methods of a new variant of the infectious bronchitis-virus (IB-virus). The new variant is also described herein as “IB80”.

### DECLARATION OF DEPOSIT

The invention provides the isolated infectious bronchitis-virus variants relating birds, shortly described as IB-viruses, as they were deposited particularly at the National Collection of Pathogenic Viruses (NCPV), Culture Collections, Public Health England, (Porton Down, Salisbury, SP4 0JG, UK) on 16 Jun. 2016 with the deposit number 16061601 and on 7 Jul. 2016 with the deposit number 16070701 according to the Budapest treaty on the international recognition of the deposit of microorganisms for the purpose of patent matters. Samples of the deposits with the mentioned numbers 16061601 and 16070701 will be provided to authorized institutions for 30 years starting from the time of deposit and for the whole life of a patent, which is granted based on this application or claims the priority of this application, in the scope of the regulations of the Budapest treaty on the international recognition of the deposit of microorganisms for the purpose of patent matters.

### BACKGROUND OF THE INVENTION

Within the previous years, significant declines in egg production were often observed in chicken flocks, which were not or only insignificantly lessened by health-promoting measures. By comprehensive observation, the suspicion was made that the decline in egg production could result from a virus, a virus which causes the infectious bronchitis.

The infectious bronchitis (IB) is a viral disease of birds, which mainly occurs in chickens and pheasants. The pathogen is the infectious bronchitis-virus (IBV), a coronavirus.

Viruses are infectious particles, which spread as virions outside of cells (extracellularly) by transmission, but are capable of reproduction as viruses only within a suitable host cell (intracellularly). All viruses have the genetic information (for their reproduction and spread), however, neither have the capability for own replication nor an own metabolism and are thus dependent on the metabolism of a host cell. Thus, virologists broadly agree to not consider viruses as living beings. Currently, approximately 3,000 virus types are identified. Viruses infect cells of eukaryotes (plants, fungi, animals, humans) and prokaryotes (bacteria and archaea).

The coronavirus is a member of the family of the coronaviridae, which is a family within the order nidovirales. The family coronaviridae is split into two sub-families and the genera Alpha-, Beta- and Gamma-, (formerly groups 1, 2 and 3 of the old genus coronavirus) and deltacoronavirus as well as torovirus and bafinovirus. The coronavirus is attributed to the sub-family coronavirinae, therein to the genus gamma-coronavirus. The species name according to ICTV (report 2015) is called: Avian coronavirus. It comprises, in addition to the infectious bronchitis virus (IBV), also the pathogen of the infectious bronchitis, for example also the former species turkey-coronavirus (TCoV), pheasant-coronavirus (PhCoV), duck-coronavirus, goose-coronavirus and pigeon-coronavirus.

Coronaviridae, with a genome length of more than 30.000 nucleotides, belong to the RNA viruses with the largest known genomes. Contrary to the typically high error rate of the RNA-polymerase of other RNA-viruses, causing a limitation of the genome length to approximately 10.000 nucleotides, a relatively high genetic stability is achieved in coronaviridae among others by a 3′-5′-exoribonuclease function of the protein Nsp-14. The single strand RNA-genome of the coronaviruses has a length of approximately 27.6000 to 31.0000 nucleotides (nt), by which coronaviruses have the longest genome of all known RNA-viruses. A 5 #-cap structure and a non-coding region (UTR, untranslated region) of approximately 200 to 400 nt, which contains a short 65 to 98 nt leader-sequence are located at the 5′-end. At the 3′-end there is a further UTR of 200 to 500 nt, which ends in a poly(A)-tail. The genome of the coronaviruses contains 6 to 14 open reading frames (ORF), of which the two biggest, the genes for the non-structure proteins 1a and 1b, are close to the 5′-end and thus slightly overlap with different reading frame. The overlap forms a hairpin structure, which enables a shift in the reading frame at the translation at the ribosomes in 20 to 30% of the reading cycles and thus leads to the synthesis of smaller amounts of the 1b-protein.

On a morphological view, the 120 to 160 nm sized viriones have a viral envelope, in which 3 or 4 different membrane proteins are incorporated: The big, glycosylated S-protein (180 to 220 kDa) as a trimer with its clubbed spikes (peplomeres) protruding to the outside for approximately 20 nm is responsible for the characteristic, wreath-like shape of the coronaviruses (lat. corona: wreath, crown). In smaller amounts, a second membrane protein E 89 to 12 kDa) is present. Within the human coronavirus OC43 and the coronaviruses of the group 2, there is additionally the HE-protein (haemagglutin-esterase-protein 65 kDa). The M-protein (23 to 35 kDa) also anchored to the envelope is directed inside and coats the inner side of the viral envelope (matrix protein). In the inside, there is a helical nucleoprotein complex. This complex consists of the nucleoprotein N (50 to 60 kDa), which is complexed with a strand of a single-strand RNA with positive polarity. Certain amino acid residues, protruding to the outside of the N-protein, interact with the matrix protein M such that the capside is associated with the inner side of the membrane.

The representatives of the virus family coronaviridae cause very different diseases in different vertebrates such as mammals, rodents, fish and birds. Coronaviruses are genetically highly variable and single virus species can also infect several host species by overcoming the species barrier.

In birds, the secretion of the virus is achieved by secretes and excretes. Entry sites of the virus are particularly the conjunctiva and mucous membranes of the upper intestine and the respiratory system. The transfer is particularly achieved by droplet infection, wherein dust particles and droplets loaded with viruses may reach long distances. The virus colonizes the ciliated epithelium of the respiratory system, but also the reproductive system and the kidneys can be affected. In poultry, the infectious bronchitis (IB) quickly spreads and particularly young animals show a high frequency of disease. The mortality varies with the virus variants. The incubation time is 18 to 36 hours. Clinically, shortness of breath, nasal discharge, stertorous breath noises, sneezing and conjunctivitis can emerge. Additionally, general disturbances with feed aversion and retention in the water absorption can be observed. Fallopian tube infections later lead to disturbances in egg production such as pale or deformed eggs with a thin shell, wind eggs, significantly reduced or completely missing laying activity (“false layers”) and reduced hatching rate. The duration of the disease as well as the clinical symptoms can vary. Often, the disease is further complicated by bacterial secondary infections. With some virus strains, a kidney infection may follow, which can lead to a high mortality by kidney failure or sepsis (toxinaemia). In case of superficial respiratory problems, chickens die due to a tracheal occlusion because of aggregation of mucus and inflammation products.

The quick spread in the stock and the clinical appearance allow a suspected diagnosis. The diagnosis can be achieved by means of pathological examination of dead birds as well as by molecular biological, serological and virological methods. Differential diagnostically, all respiratory diseases or, respectively, pathogens infecting the urogenital tract are possible. Viral infections such as infectious laryngotracheitis, Newcastle-disease and the egg-drop-syndrome but also bacterial infections such as mycoplasmosis or the avian infectious coryza as well as non-infectious diseases (e.g. feed mistakes, management mistakes) are to be distinguished. A treatment of the infectious bronchitis can only be obtained symptomatically. The control is thus based on the prophylactic vaccination which may already be performed immediately after hatching of the chicks (broilers) or, respectively, in the first weeks of life of the animals. Depending on the infection pressure, the vaccination in risk areas is to be repeated periodically according to vaccine manuals, where necessary also by means of different vaccine variants.

Currently, the strategy of control and prophylaxis seems to be improvable due to frequently occurring infections with the infectious bronchitis virus, also in vaccine stocks. The keeping of poultry in the rearing and production phase is still a big challenge due to the presence of the infectious bronchitis. The infection of a stock is of particular relevance with regard to the animal health and thus the general wellbeing of the animals as well as with regard to economical aspects. As described previously, the infectious bronchitis (IB) is an acute and highly infectious disease of the respiratory system of chickens. The disease is caused by the infectious bronchitis virus (IBV), a coronavirus, and is characterized by respiratory symptoms and symptoms relating to the urogenital tract, including gasping, sneezing, tracheal rattling and nasal discharge. In young chickens, severe shortness of breath and nephritis may occur. In laying hens, shortness of breath, (severe) redecrease of egg production and the loss of the inner egg quality (aqueous protein) and the outer egg quality (pale eggs (from brown layers), eggs with thin shell, soft, deformed, rough shalles, missing shells (wind eggs)) can be observed.

With regard to the aetiology of the infectious bronchitis virus it is to be mentioned that IBV is the first mentioned coronavirus and strongly varies in a genetic and phenotypical manner, with hundreds of described serotypes and strains. Coronaviruses have the biggest known viral RNA-genome according to the nucleotide number, with approximately 30.000 bases. The RNA forms a single strand and a single element. The IBV diversity is based on a mistake of the transcription, which is of high relevance, if it occurs in genomic sequences which encode proteins which are involved in the attachment to the host cell or in inducing of immune responses. Due to transcriptional mistakes, variants may arise which have an evolutional advantage for this variant in chickens prone to disease. Large genomic changes may occur, e.g. with a complete gene exchange, by reassortment during replication, wherein multiple subgenomic mRNAs are produced and enable reassortment during co-infections.

The present IBV problem can be summarized as follows:

The infectious bronchitis virus (IBV) is the pathogen of an acute and highly infectious disease which infects chickens of any age and represents a large economical burden. The virus has a broad spectrum of antigenetically and genetically different virus types, which complicates prevention and control of this pathogen. The IB-virus is mainly found in chicks. Further, the IB-virus or IBV similar and other avian coronaviruses can be present in pets and wild animals, including domestic fowl, partridges, geese, pigeons, guinea hens, teals, ducks and peacocks (Cavanagh, D., 2005, Coronaviruses in poultry and other birds, Avian Pathol. 34 (6), 439-448; Cavanagh, D., 2007, Coronavirus avian infection bronchitis virus, Vet. Res. 38(2), 281-297).

The IBV is a single-strand positively oriented RNA-virus of the family of coronaviridae, genus gammacoronavirus (Cavanagh and Naqi, 2003; International Committee on Taxonomy von Viren, http://www.ictvonline.org/virustaxonomy.asp). The viral genome has two untranslated regions (UTRs) at the 5′- and 3′-end (Boursnell et al, 1987; Ziebuhr et al, 2000), two overlapping open reading frames (ORFs) coding for the polyproteins 1a and 1ab and regions which encode the important structural proteins—spike (s), envelope (e), membrane (m) and nucleocapside (n) (Spaan et al, 1988; Sutou et al., 1988). Further, two accessory genes, ORF3 and ORF5, coding for the proteins 3a and 3b were described (Casais et al, 2005; Hodgson et al, 2006; Lai and Cavanagh, 1997). The S-protein (-3462 nt) localized in the surface of the viral membrane, is the main trigger for neutralizing antibodies (Cavanagh and Naqi, 1997; Winter et al., 2008) and is responsible for the viral binding and the invasion into the host cells (Cavanagh et al, 1986a; Koch et al, 1990; Niesters et al, 1987). It is posttranslationally cleaved at a cleavage site with multiple alkaline amino acids (Cavanagh et al., 1986b) into the amino-terminal S1- (-535 amino acids) and the carboxy-terminal S2 subunits (-627 amino acids).

The observation that IB serotypes can differ in the genomic area in 20% to 25% and in up to 50% in the amino acids of the S1-protein (Cavanagh et al., 2005) has drawn much attention (Cavanagh and Gelb 2008). Such variability can lead to important biological differences between strains and new serotype variants can arise as a result of a restricted amount of amino acid changes in the Spike protein. Nucleotide-heterogeneity is widely distributed in the S1-part of the S-gene and is largely present within three different hypervariable regions (HVRS) of the S1-gene (aa 38-67, 91-141 and 274-387) (Cavanagh et al, 1988; häufigsten Moore et al., 1997). Likely, the analysis of the complete or partial S1 gene nucleotide sequence was typically used to determine viral genetic types.

Currently, more than 50 different antigenetic and genetic types of IBV are recognized, several with severe economical effects on animal farming and some restricted to certain geographic regions (de Wit et al, 2011a; Jackwood, 2012). Effective monitoring is mainly based on the identification of the disease causing virus type (Jackwood and de Wit, 2013). A plurality of methods was developed to differentiate IBV-strains. Systems examining the antigenetic or genetic properties of an isolate allow the description of serotypes and genotypes, while methods directed to the immune response of chickens against an IBV-strain lead to the definition of protector types (Lohr, 1988). However, it is of particular relevance that the approaches based on genotype, serotype or protector type do not always classify the IB-viruses in the same way. Due to the lack of quick and suitable biological assays for classification of IBV, analyses of the S1-sequence data is the most used method to classify IBV-strains to groups, which are currently and apparently arbitrarily classified into genetic types, genotypes, classes and clusters.

The infectious bronchitis virus (IBV) is the pathogen of a highly infectious disease in the global poultry industry, which causes severe economical loss. The virus exists in a plurality of genetically different virus types. Phylogenetic analysis as well as measurements of the pairwise similarity between nucleotide or amino acid sequences were used to classify IBV-strains. Up to recently, there was no consent on the method with which IBV-stains are to be compared. Heterogenic designations of genetic groups which are incompatible with phylogenetic history were accepted which led to a confusing coexistence of several systems of genotyping.

The acceptance of an international recognized viral nomenclature is, however, of particular meaning for the performance of sound studies and to obtain and evaluate new results regarding epidemiology and evolution of IBV. To solve the problem of the prior art as previously described, Valastro et al. have developed a classification scheme published in 2016 (Valastro V., Holmes E. C., Britton P., Fusaro A., Jackwood M. W., Cattoli G., Monne I., S1 gene-based phylogeny of infectious bronchitis virus: An attempt to harmonize virus classification, Infection, Genetics and Evolution, 39 (2016), 349-364) and based on phylogenetic relations (based on the S1-gene), which can be updated and revised as soon as new S1-sequences are present. Valastro et al. describe a simple and reproducible phylogeny-based classification system, combined with an explicit and rational nomenclature of the line of ancestry for the allocation of IBV-strains. By using complete S1-gene sequences, these could be compared with each other and percentage similarities and divergences to each other could be determined. The sequence analyses resulted in a classification into 6 genotypes wherein a limit of approximately 30% difference in sequence between the single IBV-strains was determined in the S1-gene, from which the strains were allocated to a new genotype. The large majority of IBV-strains belongs to the genotype I and in turn forms sub-clusters within this genotype, which are denoted as lines within a genotype. For genotype I, 27 lines in total were described, for the genotypes II-VI a single line was described for each. In addition to the genotypes and lines, single S1-gene sequences of IBV-strains are called UV (unique variant). These, could not be allocated to a defined line. Due to the high rate of variability within the IB-viruses, Valastro et al. suggest that the conclusion on the phylogenetic relations alone is a criterion which is better suited for the sequence classification than pairwise sequence comparisons, as the classification scheme presented by Valastro et al. can be updated and revised when finding new S1-sequences.

With regard to the problems caused by the infectious bronchitis virus when keeping poultry in prior art, particularly in the rearing and production phase as particularly also due to the infections often also occurring in vaccinated stocks, the object of the present invention was to provide a basis for the strategy of verification, control or, respectively, prophylaxis, wherein particularly the new IBV-variant (IB80) as disclosed herein shall be affected as pathogen.

The present invention thus relates to a significant progress in the solution of the special problems caused by the IB80-virus in keeping poultry, with the aim to provide faster, more reliable and more effective agents for control, particularly diagnosis, prevention and/or treatment of infectious bronchitis in birds.

### BACKGROUND OF THE INVENTION

The object of the present invention is solved by the provision of the agent mentioned in the claims, such as the IB-virus (IB80) according to the invention, the nucleic acid according to the invention, the protein according to the invention, the vector, antibody, vaccine according to the invention, the use of the IB-virus according to the invention mentioned in the claims and the pharmaceutical mixture according to the invention.

Accordingly, the described new agents, the uses, the mixtures and the methods according to the invention are directed to a new variant IB80 and its closest relatives, wherein the agents, uses, mixtures and methods according to the invention are suitable for the diagnosis and prevention of infections with the IB80-virus, including the involved development of in-vitro-diagnostics, vaccines and for the prevention of infectious bronchitis and thus symptoms and diseases associated therewith as described further down.

The present invention is based on the isolation and identification of a new IB-virus (IB80) in birds, particularly chickens. This IB-virus (including the variants according to the invention) is also described as “IB80” within this text. IB80 was isolated from the material obtained from a chicken stock which was clinically inconspicuous in that time during a monitoring approach. For culturing, a pool of caecal tonsils was used. This organ material was homogenized in 1×PBS with gentamycin and amphotericin B at room temperature, subsequently centrifuged and the supernatant was filtered with a 0.45 μm filter. Subsequently an incubation of the filtrate at room temperature was performed. Corresponding methods and the conditions for isolation of virus material to be applied are known to the person skilled on this field.

The pre-isolated virus is a member of the family of coronaviridae, a family of RNA-viruses with the largest known genomes. Accordingly, the invention relates to the isolated IB-virus, which morphologically and phylogenetically belongs known members of the sub-family coronavirinae and, therein to the genus gamma-coronavirus, of the species avian coronavirus. As described previously, the infectious bronchitis (IB) is a viral disease mainly infecting chickens and pheasants and wherein the pathogen is the infectious bronchitis-virus (IBV), a coronavirus. For further illustration in the scope of the present invention, it is referred to the terms and descriptions to “virus”, “coronavirus”, “genome”, “RNA-viruses”, “morphology”, “infectious bronchitis”, “aetiology of the infectious bronchitis virus”, “IB serotypes”, “genotype, serotype or protector-type based approaches for classification of IB-viruses”, “viral nomenclature”, “phylogeny-based classification system” as well as “S1-gene” or, respectively, “S1-gene sequences”, on which for easier comprehension it is explicitly referred.

### Short Description of the Sequences According to the Invention:

| Sequence | |
|---|---|
| ID NO: | Sequence |
| 1 | 5′UTR |
| 2 | Gene 1ab |
| 3 | Gene S |
| 4 | Gene 3 |
| 5 | Gene M |
| 6 | Non-coding region |
| 7 | Gene 5 |
| 8 | Gene N |
| 9 | 3′UTR |
| 10 | Virus complete |
| 11 | Prot 1a |
| 12 | Prot 1b |
| 13 | Prot S |
| 14 | Prot 3a |
| 15 | Prot 3b |
| 16 | Prot 3c |
| 17 | Prot M |
| 18 | Prot 5a |
| 19 | Prot 5b |
| 20 | Prot N |

In one aspect, the invention thus relates to an isolated IB-virus affecting birds and particularly chickens, as described and defined herein. Particularly, the invention relates to an isolated IB-virus, corresponding to the deposit number 16061601 (16 Jun. 2016) or the deposit number 16070701 (7 Jul. 2016), wherein these deposits, as mentioned at “declaration of deposit” above, was made at the National Collection of Pathogenic Viruses (NCPV), Culture Collections, Public Health England, (Porton Down, Salisbury, SP4 0JG, UK).

In a further aspect, the invention relates to an isolated IB-virus comprising
a) one or more nucleic acid sections selected from the group consisting of nucleic acids of the sequences SEQ ID NOs: 1, 2, 3, 4, 5, 6, 7, 8 and 9
c) one or more proteins, comprising or consisting of an amino acid chain selected from the group of the amino acid sequences SEQ ID NOs: 11, 12, 13, 14, 15, 16, 17, 18, 19 and 20,
d) a nucleic acid, comprising or consisting of a nucleic acid section with an identity of ≥85% to SEQ ID NO: 3 or with an identity of ≥98% to one of the SEQ ID NOs: 1, 2, 4, 5, 6, 7, 8, 9 or 10 and/or
e) one or more proteins, comprising or consisting of an amino acid chain with an identity of ≥85% to the amino acid sequence SEQ ID NO: 13 or to an amino acid sequence selected from the group of amino acid sequences with an identity of ≥98% to one of the amino acid sequences SEQ ID NOs: 11, 12, 14, 15, 16, 17, 18, 19 or 20.

In the previous aspect, the invention also relates to an isolated IB-virus preferably comprising as d) a nucleic acid comprising or consisting of a nucleic acid section with an identity of ≥90%, further preferably of ≥95%, even further preferably of ≥98% and especially preferably of ≥99% to SEQ ID NO: 3, and/or preferably a nucleic acid comprising or consisting of a nucleic acid section with an identity of ≥99% to one of the SEQ ID NOs: 1, 2, 4, 5, 6, 7, 8, 9 or 10.

In the previous section, the invention also relates to an isolated IB-virus preferably comprising as e) one or more proteins comprising or consisting of an amino acid chain with an identity of ≥90%, further preferably of ≥95%, even further preferably of ≥98% and especially preferably of ≥99% to the amino acid sequence SEQ ID NO: 13, and/or preferably to an amino acid sequence selected from the group of amino acid sequences with an identity of ≥99% to one of the amino acid sequences SEQ ID NOs: 11, 12, 14, 15, 16, 17, 18, 19 or 20.

In one embodiment of the invention, an isolated IB-virus as described as preferred above can be dead or attenuated (weakened) or vital.

In a further aspect the invention relates to the complete genomic sequence of the isolated IB-virus according to the invention affecting birds and particularly chickens.

In a related aspect, the invention also relates to nucleic acid molecules as defined previously and isolated from the IB-virus according to the invention, or fragments thereof.

In a further aspect the invention also relates to, as mentioned previously, proteins or polypeptides isolated from the IB-virus according to the invention, as defined previously, and/or encoded by a nucleic acid as defined above, including viral proteins which are isolated from cells infected with the IB-virus, but not present in comparable non-infected cells; or fragments thereof.

In a further aspect the invention relates to a vector comprising or consisting of a nucleic acid section as defined above.

Furthermore, the invention relates in one aspect to an isolated antibody or an isolated antigen binding fragment thereof which binds immunospecifically to a virus, a protein or an nucleic acid as these are defined above.

Further the invention relates in one aspect to a vaccine comprising a therapeutically and prophylactically active amount of an IB-virus as defined above, or a therapeutically and/or prophylactically active amount of one or more nucleic acids as defined above and/or one or more proteins as defined above and a pharmaceutically acceptable carrier.

In the scope of the present invention, the term “vaccine” comprises, in addition to the biological, therapeutic and/or preventive function of vaccines, particularly their whole regulatory range as for example registered vaccines, vaccines which do not require registration, including particularly so-called autogenous and/or stock-specific vaccines and/or vaccines within the development and registration procedure.

A vaccination, also called protective vaccination, is the administration of a vaccine with the aim to protect from an (infectious) disease. It serves to activate the immune system towards specific substances. Vaccines were e.g. developed as precautious measure against infectious diseases. A preventive measure against an infectious disease is based on a specific, active immunisation against the pathogen and is sometimes thus also called active vaccination. Aim of the active vaccination is to enable the endogenous immune system to react to an infection with the pathogen as quickly and effective such that no or only a mild infectious disease arises therefrom. It is distinguished between attenuated vaccines (live vaccines; weakened vaccines) ad inactive vaccines (dead vaccines); toxoid vaccines belong to the latter of these. Contrarily, the passive immunisation (also healing vaccination) is only a passive immunisation by application of antibodies.

The mechanism and effectiveness of vaccines shall be quickly described in the following. Depending on the vaccine and mode of immunisation (passive or active immunisation), different application methods are applied for the administration of vaccines: Active vaccinations are administered parenterally (circumventing the gastrointestinal tract) with a syringe. It is to be distinguished between intradermal (into the skin) and intramuscular (into the muscle) injections.

The intradermal vaccination can also be performed with a lancet or a vaccination gun. For a few immunisations, the vaccine was or, respectively, is administered orally (into the mouth, oral vaccination) or nasally (into the nose) or also transdermally with a skin patch. The majority of active vaccinations is administered intramuscularly. In the economic poultry keeping, the application of living vaccines is achieved via the drinking water, via a spray (aerosol) or via the administration of eye drops (eye drop approach). Inactive vaccines are mostly applied intramuscularly.

The active vaccination corresponds to the vaccination in the medical sense and is based on an active immunisation. Therein, the immune system is stimulated to form an immune competence specific for the pathogen, without having to suffer the infectious disease itself. Live or dead vaccines are used for this purpose. The live vaccine contains attenuated but still reproducible pathogens which do not trigger the disease in the immunocompetent vaccinated subject. In case of a dead vaccine, the pathogens were killed; or are only present as fragments of the pathogen. After the vaccine entering the body, its proteins and/or sugar molecules are recognized as exogenous antigens by lymphocytes circulating in the blood and/or by tissue lymphocytes. A primary immune response by pathogen specific imprints of immunocompetent lymphocytes in form of long-living memory cells succeeds. Decisively for the protection in case of a later infection is that the antigens of the vaccination mostly equal those of the pathogen to the body. In case of an infection, the memory cells recognize the antigens of the earlier vaccine on the invaded pathogen and effect that lymphocytes differentiate one the one side to short-living plasma cells producing antibodies and on the other side to T-lymphocytes and NK-cells which represent the cellular response. The vaccination shall effect the maintenance of an immunity against the pathogen such that after infection an infectious disease is not arising, due to a specific and fast induced immune response. Toxoid vaccinations, which only contain the biologically inactive component (toxoid) of the toxin of a pathogen (as e.g. the tetanus toxoid) also belong to the dead vaccines. These do not reduce the propagation of the pathogens in the body. In case of an infection, they do not interrupt the infection, however, prevent the outbreak of the clinical disease in the vaccinated subject in such a way that the toxins of the pathogen do not become effective. Different living vaccines can be administered either simultaneously or with a distance of several weeks. For dead vaccines, also in combination with live vaccines, it applies similarly. The administrations should be based on the manufacturer's protocol.

A passive immunisation is induced if there is the danger of suffering from a severe infectious disease when contacted with the pathogen, however, the immune system of the affected subject could not deal with the pathogen neither with regard to a previous immunisation nor by an earlier infection (also without symptoms, “stille Feiung”) and thus the infected subject is not protected. In this case, an immune serum is injected to the affected subject, which contains antibodies against the pathogen in high concentrations. As the immune system, in this case, does not actively produce own antibodies, but these are applied from the outside, it is a passive immunisation of the vaccinated subject. Therefore, preferably monoclonal antibodies genetically engineered on cell cultures or if such are not present, extracts from the blood of subjects which already suffered from the corresponding infectious disease or from the blood of animals which were directly infected with the pathogen (reconvalescent serum). The passive immunisation is thus an emergency procedure in the sense of a post-exposition prophylaxis.

The advantage of immune sera is the fast-onset protection: The antibodies do not need to be produced in one to two weeks but are directly present after the injection of the immune serum for repelling the infection. The disadvantage is that the protection only lasts for weeks. Afterwards, the administered antibodies are degraded by the recipient and its organism is again at the risk of another infection with the same pathogen. This is traced back to the fact that the immune system is not stimulated by the administration of immune sera to produce an own immune memory towards the pathogen by forming memory cells. In case the immune serum is from an animal or a human, there is as further disadvantage that apart from the desired antibodies, traces of exogenous protein or polysaccharides of the donor may be present. The immune system of the recipient then evokes a cascade of immunological reactions against these exogenous components. This effects that the antibodies enriched in the immune serum are removed faster and are thus effective shorter than desired. In case of repeated administration of exogenous serum, especially of the same animal type, additionally, undesired allergic reactions of the recipient on form of a serum disease or of an allergic shock may occur. Thus, such immune sear are re placed by monoclonal antibodies if possible.

In another aspect, the invention relates to the use of an IB-virus (IB80) according to the invention as defined above, or of one or more nucleic acids as defined above and/or of one or more proteins as defined above, for the manufacture of a vaccine for prevention of symptoms or diseases caused by a virus as defined above. The symptoms or clinical disease outcome relate to the entry sites or, respectively, replication sites of the virus, thus especially conjunctiva and mucous membranes of the upper gastrointestinal and respiratory tract as well as the ciliated epithelium of the respiratory tract, the reproduction system and the kidney. Clinically, shortness of breath, nasal discharge, rattling breathing sounds, sneezing and conjunctivitis are to be mentioned. Additionally, the symptoms of the disease comprise general disturbances with feed aversion, retention in the water absorption, fallopian tube infections, laying disturbances as pale deformed eggs with thin shell, wind eggs, significantly reduced or completely missing laying activity (“false layers”) and reduced hatching rate. A possible kidney infection can, due to renal failure or sepsis resulting therefrom, lead to the death of the affected animals, whereas in case of superficial severe breathing problems, a tracheal occlusion due to aggregation of mucus and further inflammation products can be the cause of death.

In a further aspect the invention relates to the use of an IB-virus as defined above or of one of more nucleic acids as defined above and/or of one or more proteins as defined above for the production of monoclonal or polyclonal antibodies.

Finally, the invention also relates to a pharmaceutical mixture comprising a therapeutically and/prophylactically active amount of an antibody or of an isolated antigen binding fragment thereof, for therapeutic or prophylactic treatment of symptoms or diseases caused by a virus as defined above.

Further aspects relating to the invention shall be explained subsequently to further illustrate the scope and meaning of the present invention.

In other aspects, the invention relates to the sue of the isolated IB80 for diagnostic and therapeutic methods. In one embodiment, the invention provides a method for detecting an antibody in a biological sample, which is immunospecific for the genus gamma-coronavirus and the species avian coronavirus, particularly for the infectious bronchitis-virus (IBV) and especially for the variant IB80, wherein among the subject-matter of the invention described herein, at least one isolated IB-virus according to the invention or one of the proteins or polypeptides according to the invention, as described herein, are used.

In a further specific embodiment, the invention provides a method for screening an anti body, which immunospecifically binds and neutralises the variant infectious bronchitis-virus (IB80). Such an antibody is suitable for a passive immunisation of immune therapy of a subject infected with IB80.

In a further aspect, the invention provides isolated antibodies or antigen binding fragments thereof, which immunospecifically bind to the infectious bronchitis-virus (IB80) as described above.

In a further aspect, the invention relates to methods for detecting the presence or the activity of the expression of IB80, or fragments thereof, in a biological material such as cells, blood, saliva, urate, faeces etc.

In a related aspect the invention relates to a method for detecting the infectious bronchi tis-virus (IB80) according to the invention to determine its present in a biological sample, wherein the method comprises: (a) contacting the sample with an agent selectively binding to an infectious bronchitis-virus (IB80); and (b) detecting whether the agent has bound to the infectious bronchitis-virus (IB80) in the sample.

In a further aspect the invention relates to a method for determining the presence of a polypeptide according to the invention in a biological sample, wherein the method comprises: (a) contacting the sample with an agent selectively binding to an infectious bronchitis-virus (IB80); and (b) detecting whether the agent has bound to the polypeptide in the sample. In a further aspect the invention relates to a method for determining the presence of a nucleic acid molecule derived from the IB80 according to the invention in a biological sample, wherein the method comprises: (a) contacting the sample with an agent selectively binding to an infectious bronchitis-virus (IB80); and (b) detecting whether the agent has bound to the nucleic acid in the sample.

In a further aspect the invention relates to a method for propagating the infectious bronchitis-virus (IB80) according to the invention in host cells, cell lines and/or egg-systems as e.g. hatching eggs, with the infectious bronchitis-virus (IB80) according to the invention, cultivating the host cells, cell lines and/or egg-systems as e.g. hatching eggs, to enable the virus to replicate and the harvesting of the resulting virions. The present invention thus also relates to host cells, cell lines and/or egg-systems as e.g. hatching eggs infected with the infectious bronchitis-virus (IB80) according to the invention. The infectious bronchitis-virus (IB80) according to the invention is the deposit at NCPV at which infectious virus (IB80) according to the invention has been deposited. Host cells or, respectively, suitable cell lines are generally available via cell banks, egg-systems such as e.g. hatching eggs are generally available via the respective producers.

In a further aspect the invention relates to a method for detecting the presence of an antibody immunospecifically binding to IB80 in a biological sample, wherein the method comprises: (a) contacting the biological sample with the host cell according to the invention; and (b) detecting the antibody bound to the host cell.

In a further aspect the invention relates to vaccine preparations comprising the infectious bronchitis-virus (IB80) according to the invention, including recombinant and chimeric forms of the virus, the nucleic acid molecules comprised by the virus or protein sub-units of the virus. The invention also relates to a vaccine formulation comprising a therapeutically or prophylactically active amount of the infectious bronchitis virus (IB80) according to the invention and a pharmaceutically acceptable carrier. In one embodiment the invention relates to a vaccine formulation comprising a therapeutically or prophylactically effective amount of a protein extract of the infectious bronchitis-virus (IB80) according to the invention or a sub-unit thereof and a pharmaceutically acceptable carrier. In a further aspect the invention relates to a vaccine formulation comprising a therapeutically or prophylactically effective amount of a nucleic acid molecule comprising one or more nucleic acid sections selected from the group consisting of nucleic acids of the sequences SEQ ID NOs: 1, 2, 3, 4, 5, 6, 7, 8 and 9; or comprising a nucleic acid of SEQ ID NO: 10; or comprising a nucleic acid comprising or consisting of a nucleic acid sequence with an identity of ≥85% to SEQ ID NO: 3 or with an identity of ≥98% to one of the SEQ ID NOs: 1, 2, 4, 5, 6, 7, 8, 9 or 10; or each a complement thereof, and a pharmaceutically acceptable carrier. Particularly for the previous SEQ ID NOs, the preferred %-identities indicated above also apply.

In a further aspect the present invention relates to a vaccine formulation with a therapeutically or prophylactically active amount of a nucleic acid molecule comprising any of the nucleic acid sequences of a complement thereof, and a pharmaceutically acceptable carrier.

In a related aspect the invention relates to an immunogenic formulation comprising an immunogenically effective amount of the infectious bronchitis-virus (IB80) according to the invention and a pharmaceutically acceptable carrier. In another related aspect the invention relates to an immunogenic formulation comprising an immunogenically effective amount of a protein extract of the infectious bronchitis-virus (IB80) according to the invention or a sub-unit thereof and a pharmaceutically acceptable carrier.

In another related aspect the invention relates to an immunogenic formulation comprising an immunogenically effective amount of a nucleic acid molecule comprising one or more nucleic acid sections selected from the group consisting of nucleic acids of the sequences SEQ IN NOs: 1, 2, 3, 4, 5, 6, 7, 8 and 9; or comprising a nucleic acid of the SEQ IF NO: 10; or comprising a nucleic acid comprising or consisting of a nucleic acid section with an identity of ≥85% to SEQ ID NO: 3 or with an identity of ≥98% to one of the SEQ ID NOs: 1, 2, 4, 5, 6, 7, 8, 9 or 10; or each a complement thereof, and a pharmaceutically acceptable carrier. In a further related aspect the invention relates to an immunogenic formulation comprising an immunogenically effective amount of a nucleic acid molecule comprising one of the other nucleic acid sequences according to the invention or a complement thereof, and a pharmaceutically acceptable carrier. In another related aspect the invention relates to an immunogenic formulation comprising an immunogenically effective amount of one of the polypeptides according to the invention.

In another aspect the present invention relates to pharmaceutical compositions comprising antiviral agents according to the present invention and a pharmaceutically acceptable carrier. In a specific embodiment the antiviral agent of the invention is an antibody immunospecifically binding to the infectious bronchitis-virus (IB80). In another specific embodiment the antiviral agent is a polypeptide or a protein of the present invention or a nucleic acid molecule of the present invention.

In a related aspect the invention relates to a pharmaceutical composition comprising a prophylactically or therapeutically effective amount of an anti-infectious bronchitis-virus (IB80) and a pharmaceutically acceptable carrier.

The invention also relates to kits containing compositions and formulations of the present invention. Thus the invention relates in another aspect a kit comprising a container containing the immunogenic formulation according to the invention. In a further aspect the invention relates to a kit comprising a container containing the vaccine formulation ac cording to the invention. In another aspect the invention relates to a kit comprising a container containing the pharmaceutical composition according to the invention. In another aspect the invention relates to a kit comprising a container containing the vaccine formulation according to the invention. In another aspect the invention relates to a method for identifying a subject infected with the infectious bronchitis-virus (IB80) according to the invention comprising: (a) obtaining the total RNA of a sample obtained from the subject; (b) reverse transcription of the total RNA to obtain cDNA; and (c) amplifying the cDNA by use of a set of primers derived from a nucleic acid sequence of the infectious bronchitis-virus (IB80) according to the invention.

The invention further relates to the use of the sequence information of the isolated infectious bronchitis-virus (IB80) according to the invention for diagnostic and therapeutic methods.

In another aspect the present invention relates to methods for screening of antiviral agents which inhibit the infection ability or the replication of infectious bronchitis-viruses (IB80) or further variants thereof.

The invention further relates to methods for producing recombinant or chimeric forms of the infectious bronchitis-virus (IB80).

### DETAILED DESCRIPTION OF THE PREFERRED EMBODIMENTS

Of course, the present invention is not restricted to certain described embodiments as such can of course vary. Of course, the terminology as used herein is only thought for the purpose of describing certain embodiments and not as a restriction.

Due to the sequence diversity of IB80 relative to all previously recorded avian viruses, the present invention is suitable for the design of diagnostic assays to monitor IB80-diseases in animal subjects, particularly birds, and to develop effective virostatic agents and vaccines.

In the S1 gene, the IB-virus of the present invention (IB80) is genetically different for ≥20% from all other known IB variants. The unique nature of this IB-virus provided challenges for the isolation traditional molecular-based diagnostic assays and approaches for the genome sequencing. However, it was completely sequenced by the inventors.

The definitions provided herein apply to the whole description of the invention disclosed herein, including the summary of the invention above.

The term “an antibody or an (antigen binding) fragment of an antibody, immunospecifically binding to a polypeptide of the invention” as used herein, describes and antibody or a fragment thereof that immunospecifically binds to a virus according to the invention, a protein according to the invention, or to the polypeptide encoded by an (IBV) nucleotide sequence, comprising one or more nucleic acid sections selected from the group consisting of nucleic acids of the sequences SEQ ID NOs: 1, 2, 3, 4, 5, 6, 7, 8 and 9; or comprising a nucleic acid of SEQ ID NO: 10; or comprising a nucleic acid comprising or consisting of a nucleic acid section with an identity of ≥85% to SEQ ID NO: 3 or with an identity of ≥98% to one of the SEQ ID NOs: 1, 2, 4, 5, 6, 7, 8, 9 or 10. The proteins associated with the term “antibody” or “(antigen binding) fragment of an antibody” according to the invention comprise one or more proteins comprising or consisting of an amino acid chain selected from the group of the amino acid sequences SEQ ID NOs: 11, 12, 13, 14, 15, 16, 17, 18, 19 ad 20; or one or more proteins comprising or consisting of an amino acid chain with an identity of ≥85% to the amino acid sequence SEQ ID NO: 13 or to an amino acid sequence selected from the group of amino acid sequences with an identity to ≥98% to one of the amino acid sequences SEQ ID NOs: 11, 12, 14, 15, 16, 17, 18, 19 or 20. Particularly, the indicated preferred %-identities also apply for the previous SEQ ID NOs. An antibody or a fragment thereof immunospecifically binding to the polypeptide according to the invention can cross-react with other antigens. Preferably, an antibody or a fragment thereof immunospecifically binding to the polypeptide according to the invention does not cross-react with other antigens. An antibody or a fragment thereof immunospecifically binding to the polypeptide according to the invention can for example be identified by immunoassays or other methods known to the person skilled on this field or by other ways as described herein.

The term “immunospecific” or, respectively the term “immunospecificity” is a term known to the skilled person and which is frequently used in connection with antibodies or, respectively antigens. Thus the term “immunospecific” or, respectively the term “immunospecificity” generally describes the ability of a substance or compound including nucleic acids, peptides, polypeptides, fragments thereof, to specifically react with an antibody molecule directed against it in the sense of and antibody-antigen-binding. This binding can be detected with immunological methods. The mentioned terms are thus in connection with the specific immune response which follows the contact with e.g. an antigen in an affected subject; the immune response herein contains, where applicable, e.g. the formation of specific T-lymphocytes in an animal subject, which specifically react with the mentioned substance or compound including nucleic acids, peptides, polypeptides, fragments thereof or, respectively, with the antigen. Preferably, the presence of an immunospecific binding is examined with an experiment as in example 5.

An “isolated” or “purified” peptide or protein is substantially free of cellular material or other contaminating proteins of the cell or the tissue source from which the protein originates, or substantially free of chemical precursors or other chemicals when synthesized chemically. The term “substantially free of cellular material” comprises preparations from a polypeptide/protein in which the polypeptide/protein is separated from cellular components of the cell out of which is was isolated or in which it was recombinantly produced. Thus a polypeptide/protein being substantially free of cellular material comprises preparations of the polypeptide/protein with less than approximately 30%, 20%, 10%, 5%, 2.5% or, respectively, 1% (depending on the dry weight) of contaminating protein. If the polypeptide/protein is produced recombinantly, it is preferably also substantially free of culture medium, i.e. the culture medium amounts to less than approximately 20%, 10% or 5% of the volume of the protein preparation. If a polypeptide/protein is chemically synthesized, it is preferably substantially free of chemical precursors or other chemicals, i.e. it is separated from chemical precursors or other chemicals, which were included in the synthesis of the protein. Accordingly, such preparations of the polypeptide/protein have less than approximately 30%, 20%, 10%, 5% (depending on the dry weight) of chemical precursors or other compounds than the polypeptide/protein of interest. In a preferred embodiment of the present invention, the polypeptides/proteins are isolated or purified.

The term “isolated” as used herein relates to a determined biological or synthetic material being separated from its natural or synthetic environment and being purified, particularly a virus, a viral component, a nucleic acid, RNA, DNA, cDNA, fragments thereof, nucleic acid sections, a protein, an amino acid sequence, fragments thereof, an antibody or an antigen binding fragment thereof, and similar material, wherein the mentioned determined material is preferably substantially free of other accompanying materials of any nature, e.g. accompanying nucleic acids, proteins, lipids, carbohydrates or other material with which the determined isolated material can generally be associated at natural or synthetic conditions, e.g. by association with another cellular material, culture medium or in a synthesis medium.

For example, an “isolated” nucleic acid molecule or a fragment thereof or a nucleic acid section is thus e.g. each one that is separated and purified from other nucleic acids molecules present in the natural source of the nucleic acid molecule. Furthermore, an “isolated” nucleic acid molecule, such as an RNA molecule, a cDNA molecule, can substantially be free of other cellular material or culture medium if it is produced by recombinant techniques or is substantially free of chemical precursors or other chemicals if chemically synthesized. In a preferred embodiment of the invention, the nucleic acid molecules coding for the polypeptides/proteins of the invention are isolated or purified. The term “isolated” nucleic acid molecule does not comprise nucleic acids and the like which are a member of a library but not separated and purified from the other nucleic acid molecules in the library.

The term “part” or “fragment” as used herein includes the mentioned fragment lengths and all numbers in between, including the endpoints of a determined range and including an arbitrary length up to the complete length of a protein, polypeptide or nucleic acid.

The term “with a biological activity of the protein” or “with biological activities of the polypeptides of the invention” relates to the attribute of the polypeptides or proteins which have a common biological activity, a similar or identical structural domain and7or a sufficient amino acid identity to the polypeptide encoded by the nucleic acid sequence comprising one or more nucleic acid sections selected from the group consisting of nucleic acids of the sequences SEQ ID NOs: 1, 2, 3, 4, 5, 6, 7, 8 and 9; or comprising a nucleic acid of SEQ ID NO: 10; or comprising a nucleic acid comprising or consisting of a nucleic acid section with an identity of ≥85% to SEQ ID NO: 3 or with an identity of ≥98% to one of the SEQ ID NOs: 1, 2, 4, 5, 6, 7, 8, 9 or 10. The proteins according to the invention being related to the term “with a biological activity of the protein” or “with biological activities of the polypeptides of the invention” comprise one or more proteins comprising or consisting of an amino acid chain selected from the group of the amino acid sequences SEQ ID NOs: 11, 12, 13, 14, 15, 16, 17, 18, 19 ad 20; or one or more proteins comprising or consisting of an amino acid chain with an identity of ≥85% to the amino acid sequence SEQ ID NO: 13 or to an amino acid sequence selected from the group of amino acid sequences with an identity to ≥98% to one of the amino acid sequences SEQ ID NOs: 11, 12, 14, 15, 16, 17, 18, 19 or 20. Particularly, the preferred %-identities described above also apply to the previous SEQ ID NOs. Such common biological activities of the polypeptides of the invention comprise antigenicity and immunogenicity.

The term “under stringent conditions” relates to the hybridization and washing conditions at which nucleotide sequences which have an identity of at least 70%, at least 75%, at least 80%, at least 85%, at least 85%, at least 90% or at least 95% sty hybridised to each other. Such hybridization conditions are e.g. described but not limited to Current Protocols in Molecular Biology, John Wiley & Sons, NY (1989), 6.3.1-6.3.6; Basic Methods in Molecular Biology, Elsevier Science Publishing Co., Inc., New York (1986), p. 75 78 and 84-87. and Molecular Cloning, Cold Spring Harbor Laboratory, NY (1982), p. 387-389) and are well known to the skilled person in this field. A preferred not limiting example for stringent hybridization conditions is the hybridization in 6× Sodium chloride/sodium citrate (SSC), 0.5% SDS at approximately 68° C., followed by one or more washing steps in 2×SSC, 0.5% SDS at room temperature. A further preferred but not limiting example for stringent hybridization conditions is a hybridization in 6×SSC at approximately 45° C. followed by one or more washing steps in 0.2×SSC, 0.1% SDS at approximately 50-65° C.

The term “variant” as used herein either relates to a naturally occurring genetic mutant of IB80 or to a recombinantly produced variation of this IBV-variant, of which each contains one or more mutations in its genome compared to the IBV which comprises one or more nucleic acid sections selected from the group consisting of nucleic acids of the sequences SEQ ID NOs: 1, 2, 3, 4, 5, 6, 7, 8 and 9; or comprising a nucleic acid of SEQ ID NO: 10; or comprising a nucleic acid comprising or consisting of a nucleic acid section with an identity of ≥85% to SEQ ID NO: 3 or with an identity of ≥98% to one of the SEQ ID NOs: 1, 2, 4, 5, 6, 7, 8, 9 or 10. Particularly, the preferred %-identities as mentioned above also apply here (as well as in the further text) for the previous SEQ ID NOs. The term “variant” can also relate to either a naturally occurring variant of a given peptide or to a recombinantly produced variation of a given peptide or protein, in which one or more amino acid residues were modified by amino acid substitution, addition or deletion.

The term “homology” relates to the sequence similarity or alternatively to the sequence identity between two or more polynucleotide sequences or two or more polypeptide sequences.

The term “percent-identity” and “%-identity” as applied to polynucleotide sequences relate to the percentage of the identical nucleotide-match between at least two polynucleotide sequences being aligned with each other with a standardised algorithm (“alignment”). Such an algorithm can, in a standardised and reproducible way, introduce gaps into the compared sequences to optimize the alignment between the two sequences and thus achieve a more meaningful comparison of the two sequences.

The percentage identity between polynucleotide sequences can be determined., by using one or more computer algorithms or programs known in the prior art and described herein. For example, the percentage identity can be determined by using the default parameters of the CLUSTAL V-algorithm as in version 3.12e as integrated into the MEGALIGN sequence alignment program. This program is part of the LASERGENE software package, a range of molecularbiological analysis programs (DNASTAR, Madison, Wis.). CLUSTAL V is described in Higgins, D. G. and P. M. Sharp (1989; CABIOS 5:151-153) and in Higgins, D. G. et al. (1992; CABIOS 8:189-191). For pairwise alignments of polynucleotide sequences, the standard parameters are set as follows: Ktuple=2, 5 Gap Penalty=5, Window=4 and “Diagonals Saved”=4. The weighted residuum weight table is selected as default setting. Particularly the CLUSTAL W-algorithm may be used.

The Clustal W algorithm is a widely spread alignment program which is suitable for alignments of more than two sequences (multi-sequence alignment). The Clustal W program uses a progressive alignment by using the neighbour-joining algorithm for generating a multiple sequence alignment.

Alternatively hereto, a series of generally used and freely accessible sequence comparison algorithms which can be used is the Basic Local Alignment Search Tool (BLAST) (Altschul, S. F. et al. (1990) J. Mol. Biol. 215:403-410) provided by the National Center for Biotechnology Information (NCBI) which is accessible from various sources including the NCBI, Bethesda, Md. And on the NCBI world wide web site in the internet. The BLAST-software-series contains various programs including the sequence analysis “blastn” which is used for aligning a known polynucleotide sequence with other polynucleotide sequences from a plurality of databases. Also accessible is a tool called “BLAST 2 sequences” which is used for the direct pairwise comparison of two nucleotide sequences. “BLAST 2 Sequences” can also be interactively accessed and used by the NCBI world wide web site in the internet. The “BLAST 2 Sequences” tool cans be used for both, blastn and blastp (discussed below). BLAST programs are typically used with gap and other parameters in default settings. For example, for comparing two nucleotide sequences, blastn can be used with the “BLAST 2 Sequences” tool version 2.0.12 (21 Apr. 2000), which is set to default settings. Such standard parameters can be for example: Matrix: BLOSUM62; Reward for Match: 1; Penalty for Mismatch: −2; Open Gap: 5 and Extension-Gap: 2 Penalties; Gap x Drop-off: 50; Expect: 10; Word Size: 11; Filter: on.

Percentage identity can be measured over the length of a total defined sequence, as e.g. by a determined SEQ ID-Number, or can be measured over a shorter length e.g. over the length of a fragment which is taken from a larger defined sequence, e.g. a fragment of at least 20, at least 30, at least 40, at least 50, at least 70, at least 100 or at least 200 sub sequent nucleotides. Such lengths are only for example and of course any fragment length based on the sequences shown in here in tables, figures or sequence lists can be used to describe a length over which the percentage identity can be measured.

The term “percent-identity” and “%-identity” as applied to polynucleotide sequences relate to the percentage of the identical nucleotide-match between at least two polynucleotide sequences being aligned with each other with a standardised algorithm. Methods for the polypeptide sequence alignment are well known. Several alignment methods take into account the conservative amino acid substitutions. Such conservative substitutions as described above generally conserve the charge and hydrophobicity at the position of the substitution by which the structure (and thus the function) of the polypeptide is maintained. The term “percent-identity” and “%-identity” as applied to polynucleotide sequences refer to the percentage of the match of the residues including identical residue matches and conservative substitutions between at least two aligned polypeptide sequences by using a standardised algorithm. Contrarily, conservative substitutions are not included in the calculation of the percentage identity between polypeptide sequences.

The percent identity between polypeptide sequences can be determined by using the default parameters of the CLUSTAL V algorithm as integrated into version 3.12e of the MEGALIGN sequence align program (as described and referred above). For pairwise alignment of polypeptide sequences by using CLUSTAL V the standard parameters are set as follows: Ktuple=1, Gap Penalty=3, Window=5, and “Diagonals Saved”=5. The PAM250 matrix is selected as standard residue weight table.

Alternatively, the NCB BLAST software-series can be used. For example for a pairwise comparison of two polypeptide sequences, the “BLAST 2 Sequences” tool of the version 2.0.12 (21 Apr. 2000) can be set to standard parameters with blastp. Such standard parameters can be for example: Matrix: BLOSUM62; Open Gap: 11 and extension-Gap: 1 Sanction; Gap x Drop-off: 50; Expect: 10; Word-Size: 3; Filter: on.

Percentage identity can be measured over the length of a total defined sequence, as e.g. by a determined SEQ ID-Number, or can be measured over a shorter length e.g. over the length of a fragment which is taken from a larger defined sequence, e.g. a fragment of at least 20, at least 30, at least 40, at least 50, at least 70, at least 100 or at least 200 sub sequent residues. Such lengths are only exemplary and of course each fragment length is based on the sequences, tables, figures or sequence lists shown herein to be used for describing a length over which the percentage identity is measured.

The term “agent” comprises a chemical, biochemical or biological molecule; as e.g. small molecules, proteins, polypeptides, antibodies, nucleic acid molecules including DNA or RNA and the like.

### Methods and Compositions Related to the IBV (IB80) According to the Invention

The present invention is based on the isolation and identification of a new variant of an infectious bronchitis-virus, IB80, and its sequencing. IB80 was isolated from chickens with infectious bronchitis. The isolated virus has an RNA single-strand and is a member of the family of Coronaviridae, a family of positively oriented RNA-viruses of the genus gamma-coronavirus, of the species avian coronavirus. Accordingly, the invention relates to the isolated IB-virus which is morphologically and phylogenetically related to known members of the coronaviridae and particularly the gammacoronaviruses.

In a further aspect the invention relates to an isolated IB-virus containing a nucleic acid molecule with a nucleotide sequence which is hybridised under stringent conditions with one of the defined sequences according to the invention, wherein the sequences according to the invention are preferably selected from the group consisting of:
one or more nucleic acid sections selected from the group consisting of nucleic acids of the sequences SEQ ID NOs: 1, 2, 3, 4, 5, 6, 7, 8 and 9;
a nucleic acid of the sequence SEQ ID NO: 10; or
a nucleic acid comprising or consisting of a nucleic acid section with an identity of ≥85% to SEQ ID NO: 3 or with an identity of ≥98% to one of the SEQ ID NOs: 1, 2, 4, 5, 6, 7, 8, 9 or 10;
   or wherein the nucleic acid molecule containing isolated IB-virus has a nucleotide sequence which has an identity of at least 70%, 75%, 80%, 85%, 90%, 95%, 96%, 97%, 98% or 99%, with the previously mentioned SEQ ID NOs.

In one embodiment of the present invention the IB-virus is killed. In another embodiment, the IB-virus is attenuated. In a further embodiment the infectivity of the attenuated IB-virus is reduced. In another, the infectivity is reduced by 5-fold, 10-fold, 25-fold, 50-fold, 100-fold, 250-fold, 500-fold or 10.000-fold. In other embodiments the replication ability of the attenuated IB-virus is reduced. In another, the replication ability of the attenuated IB-virus is reduced by 5-fold, 10-fold, 25-fold, 50-fold, 100-fold, 250-fold, 500-fold, 1.000-fold or 10.000-fold. In another, the ability of the attenuated IB-virus for protein synthesis is reduced. In another, the ability of the attenuated IB-virus for protein synthesis is reduced by 5-fold, 10-fold, 25-fold, 50-fold, 100-fold, 250-fold, 500-fold, 1.000-fold or 10.000-fold.

In another, the ability of the attenuated IB-virus for assembly is reduced. In another, the ability of the attenuated IB-virus for assembly is reduced by 5-fold, 10-fold, 25-fold, 50-fold, 100-fold, 250-fold, 500-fold, 1.000-fold or 10.000-fold. In another, the cytopathic effect of the attenuated IB-virus for protein synthesis is reduced. In another, the cytopathic effect of the attenuated IB-virus for protein synthesis is reduced by 5-fold, 10-fold, 25-fold, 50-fold, 100-fold, 250-fold, 500-fold, 1.000-fold or 10.000-fold.

In a further aspect, the invention provides the complete genomic sequence of the IB-virus. In a special embodiment, the IB-virus comprises a nucleotide sequence of SEQ ID NO: 10, which corresponds to the complete virus.

In a related aspect, the invention relates to nucleic acid molecule isolated from IBV or fragments thereof. In one embodiment of the present invention, the isolated nucleic acid molecule comprises one or more nucleic acid sections selected from the group consisting of nucleic acids of the sequences SEQ ID NOs: 1, 2, 3, 4, 5, 6, 7, 8 and 9; or comprising a nucleic acid of SEQ ID NO: 10; or comprising a nucleic acid comprising or consisting of a nucleic acid section with an identity of ≥85% to SEQ ID NO: 3 or with an identity of ≥98% to one of the SEQ ID NOs: 1, 2, 4, 5, 6, 7, 8, 9 or 10; or a complement thereof. Particularly, the %-identities mentioned above also apply for the previous SEQ ID NOs. In another, the nucleic acid molecule comprises a nucleic acid sequence with at least 4, 5, 10, 15, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1500, 2000, 2500, 3000, 3500, 4000, 4500, 4600, 4700, 4800 or 4900 connected nucleotides of the nucleotide sequence of SEQ ID NO: 10 or a complement thereof; or at least 5000, 5500, 5600, 5700, 5800, 5900, 6000, 6100, 6200, 6300, 6400, 6500 or 6600 be neighboured nucleotides of the nucleotide sequence of SEQ ID NO: 10 or a complement thereof. In a further embodiment, the isolated nucleic acid molecule comprises a nucleotide sequence encodes the IBV-amino acid sequence of one or more proteins comprising or consisting of an amino acid chain selected from the group of the amino acid sequences SEQ ID NOs: 11, 12, 13, 14, 15, 16, 17, 18, 19 ad 20; or one or more proteins comprising or consisting of an amino acid chain with an identity of ≥85% to the amino acid sequence SEQ ID NO: 13 or to an amino acid sequence selected from the group of amino acid sequences with an identity to ≥98% to one of the amino acid sequences SEQ ID NOs: 11, 12, 14, 15, 16, 17, 18, 19 or 20; or a complement of the nucleotide sequence which encodes the IBV-amino acid sequences of the previously mentioned SEQ ID NOs. In another, the isolated nucleic acid molecule hybridises under stringent conditions to a nucleic acid molecule with the nucleotide sequence of SEQ ID NOs: comprising or consisting of one or more nucleic acid sections selected from the group consisting of nucleic acids of the sequences SEC ID NOs: 1, 2, 3, 4, 5, 6, 7, 8 and 9; or comprising a nucleic acid of SEQ ID NO: 10; or comprising a nucleic acid comprising or consisting of a nucleic acid section with an identity of ≥85% to SEQ ID NO: 3 or with an identity of ≥98% to one of the SEQ ID NOs: 1, 2, 4, 5, 6, 7, 8, 9 or 10; or a complement thereof, wherein the hybridising nucleic acid molecule encodes an amino acid sequence which has a biological activity which is shown by a polypeptide which is encoded by a nucleotide sequence of the previous SEQ ID NOs. Particularly, the above mentioned %-identities also apply for the previous SEQ ID NOs. In another, the nucleic acid molecule is RNA. In another, the nucleic acid molecule is DNA.

In a further aspect, the invention relates to proteins or polypeptides including viral proteins which are isolated from cells infected with the IS-virus such as host cells, cell lines and/or egg-systems such as hatching eggs, however which are not present in comparable non-infected cells such as host cells, cell lines and/or egg-systems such as hatching eggs. In one embodiment of the present invention, the amino acid sequences of one or more proteins comprising or consisting of an amino acid chain selected from the group of the amino acid sequences SEQ ID NOs: 11, 12, 13, 14, 15, 16, 17, 18, 19 ad 20; or one or more proteins comprising or consisting of an amino acid chain with an identity of ≥85% to the amino acid sequence SEQ ID NO: 13 or to an amino acid sequence selected from the group of amino acid sequences with an identity to ≥98% to one of the amino acid sequences SEQ ID NOs: 11, 12, 14, 15, 16, 17, 18, 19 or 20; or a fragment thereof are provided. In one embodiment, the polypeptides or proteins of the present invention have a biological activity of the protein (including antigenicity and/or immunogenicity) which is encoded by the sequence comprising one or more nucleic acid sections selected from the group consisting of nucleic acids of the sequences SEC ID NOs: 1, 2, 3, 4, 5, 6, 7, 8 and 9; or comprising a nucleic acid of SEQ ID NO: 10; or comprising a nucleic acid comprising or consisting of a nucleic acid section with an identity of ≥85% to SEQ ID NO: 3 or with an identity of ≥98% to one of the SEQ ID NOs: 1, 2, 4, 5, 6, 7, 8, 9 or 10. In another, the polypeptides or proteins of the present invention have a biological activity of at least one protein with the amino acid sequence (including antigenicity and/or immunogenicity) as this amino acid sequence or a fragment thereof is presented herein. Particularly, the above mentioned %-identities also apply for the previous SEQ ID NOs.

In a related aspect, the present invention relates to an isolated polypeptide which is encoded by the nucleic acid molecule of the invention des cribbed above. In one embodiment of the present invention, the isolated polypeptide comprises the amino acid sequence selected from the group consisting of a) one or more proteins comprising or consisting of an amino acid chain selected from the group of the amino acid sequences SEQ ID NOs: 11, 12, 13, 14, 15, 16, 17, 18, 19 ad 20; or one or more proteins comprising or consisting of an amino acid chain with an identity of ≥85% to the amino acid sequence SEQ ID NO: 13 or to an amino acid sequence selected from the group of amino acid sequences with an identity to ≥98% to one of the amino acid sequences SEQ ID NOs: 11, 12, 14, 15, 16, 17, 18, 19 or 20; and b) an amino acid sequence which has an identity of at least 70%, 75%, 80%, 85%, 90%, 95%, 96%, 97%, 98% or 99%, with the amino acid sequence according to a). In another, the isolated polypeptide comprises the amino acid sequence with at least 5, 10, 15, 20, 25, 30, 35, 40, 45, 50, 60, 70, 80, 90, 100, 150, 200, 210, 220, 230, 240, 250, 260, 270, 280, 290, 300, 310, 320, 330, 340, 350, 360, 370, 380, 390, 400, 450, 500, 550, 600, 610, 620, 630, 640, 650, 660, 670, 700, 710, 720, 730, 750, 800, 850, 900, 950, 1000, 1050, 1100, 1150, 1200, 1250, 1300, 1350, 1400, 1450, 1500, 1550, 1600, 1650, 1700, 1750, 1800 1850, 1900, 1950, 2000, 2050, 2100, 2150, 2160, 2170, 2180, 2190 or 2200 connected amino acid residues of the amino acid sequence of SEQ ID NOs presented previously.

In other aspects, the invention relates to the use of an isolated IB-virus for diagnostic and therapeutic methods. In one embodiment, the invention provides a method for detecting an antibody which is immunospecific in a biological sample for the IB-virus, by use of the isolated IB-virus as described above and according to the invention or for any of the proteins or polypeptides described herein and according to the invention. In another specific embodiment, the invention relates to a method for screening for an antibody which binds immunospecifically and neutralises IBV or a combination of IBVs. Such an antibody is suitable for a passive immunisation of immunotherapy of a subject infected with IBV.

In a further aspect, the invention relates to an isolated antibody or an antigen binding fragment thereof, which immunospecifically binds to one of the IB-virus-genera of the invention as described above. In one embodiment of the present invention, the isolated antibody or an antigen binding fragment thereof neutralises a genus of the IB-virus. In another, the isolated antibody or an antigen binding fragment thereof immunospecifically binds to the polypeptide according to the invention described above. The invention further relates to antibodies, which specifically bind to a polypeptide of the invention, which is encoded by the nucleotide sequence comprising one or more nucleic acid sections selected from the group consisting of nucleic acids of the sequences SEC ID NOs: 1, 2, 3, 4, 5, 6, 7, 8 and 9; or comprising a nucleic acid of SEQ ID NO: 10; or comprising a nucleic acid comprising or consisting of a nucleic acid section with an identity of ≥85% to SEQ ID NO: 3 or with an identity of ≥98% to one of the SEQ ID NOs: 1, 2, 4, 5, 6, 7, 8, 9 or 10, a fragment thereof or which is encoded by a nucleic acid comprising a nucleotide sequence which hybridises under stringent conditions to one of the previously described nucleotide sequences and/or to any IB80 epitope with one or more biological activities of a polypeptide of the invention. These polypeptides comprise the proteins according to the invention, i.e. one or more proteins comprising or consisting of an amino acid chain selected from the group of the amino acid sequences SEQ ID NOs: 11, 12, 13, 14, 15, 16, 17, 18, 19 ad 20; or one or more proteins comprising or consisting of an amino acid chain with an identity of ≥85% to the amino acid sequence SEQ ID NO: 13 or to an amino acid sequence selected from the group of amino acid sequences with an identity to ≥98% to one of the amino acid sequences SEQ ID NOs: 11, 12, 14, 15, 16, 17, 18, 19 or 20. Particularly, the above mentioned %-identities also apply for the previous SEQ ID NOs. Such antibodies include, but are not restricted thereon, polyclonal, monoclonal, bispecific, multispecific, human, humanized, chimeric antibodies, single chain antibodies, Fab-fragments, F(ab′)₂-fragments, disulfide connected Fvs, intrabodies and fragments containing either a VL or VH domain or even a complementarity determining region, which specifically binds to a polypeptide of the invention.

In other aspects, the invention relates to methods for detecting the presence of the activity or the expression of the IB-virus of the invention in a biological material such as cells, blood, saliva, urine, and the like. The increased or reduced activity or expression of the IB-virus in a sample, relative to a control sample, can be determined by contacting the biological material to an agent which can directly or indirectly recognise the presence, the activity or the expression of the IB-virus. In one embodiment of the present invention, the antibodies or nucleic acid molecules of the invention are selected as such detection agents.

In a related aspect, the invention relates to a method for detecting the presence of the IB-virus according to the invention and described above in a biological sample, wherein the method comprises: (a) contacting the sample with an agent, which selectively binds to the IB-virus; and (b) detecting whether the agent binds to the IB-virus in the sample. In one embodiment of the present invention, the biological sample is selected from the group consisting of cells; blood; serum; plasma; faeces; tracheal, choanal or cloacal and con junctiva smears. In another, the agent which binds to the IB-virus is an antibody. In an other, the agent which binds to the IB-virus is a nucleic acid molecule comprising the nucleotide sequence comprising one or more nucleic acid sections selected from the group consisting of nucleic acids of the sequences SEC ID NOs: 1, 2, 3, 4, 5, 6, 7, 8 and 9; or comprising a nucleic acid of SEQ ID NO: 10; or comprising a nucleic acid comprising or consisting of a nucleic acid section with an identity of ≥85% to SEQ ID NO: 3 or with an identity of ≥98% to one of the SEQ ID NOs: 1, 2, 4, 5, 6, 7, 8, 9 or 10; or a complement thereof. Particularly, the above mentioned %-identities also apply for the previous SEQ ID NOs. In another, the agent binding to the IB-virus is a nucleic acid molecule comprising a nucleotide sequence with at least 4, 5, 10, 15, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1500, 2000, 2500, 3000, 3500, 4000, 4500, 4600, 4700, 4800, 4900, 5000, 5500, 5600, 5700, 5800, 5900, 6000, 6100, 6200, 6300, 6400, 6500 or 6600 neighboured nucleotides of the nucleotide sequence of one of the previously mentioned SEQ ID NOs.

In a further aspect, the invention relates to a method for determining the presence of the polypeptide according to the invention and as described above in a biological sample, wherein the method comprises: (a) contacting the sample with an agent, which selectively binds to the IB-virus; and (b) detecting whether the agent binds to the IB-virus in the sample. In one embodiment of the present invention, the biological sample is selected from the group consisting of cells; blood; serum; plasma; faeces; tracheal, choanal or cloacal and conjunctiva smears. In another, the agent which binds to the IB-virus is an antibody or an antigen binding fragment thereof.

In a further aspect, the invention relates to a method for detecting the presence of a first nucleic acid molecule which is derived from the IB-virus according to the invention and as described above in a biological sample, wherein the method comprises: (a) contacting the sample with an agent, which selectively binds to the IB-virus; and (b) detecting whether the agent binds to the IB-virus in the sample.

In one embodiment of the present invention, the agent binding to the first nucleic acid molecule is a second nucleic acid molecule comprising the nucleotide sequence comprising one or more nucleic acid sections selected from the group consisting of nucleic acids of the sequences SEC ID NOs: 1, 2, 3, 4, 5, 6, 7, 8 and 9; or comprising a nucleic acid of SEQ ID NO: 10; or comprising a nucleic acid comprising or consisting of a nucleic acid section with an identity of ≥85% to SEQ ID NO: 3 or with an identity of ≥98% to one of the SEQ ID NOs: 1, 2, 4, 5, 6, 7, 8, 9 or 10; or a complement thereof. Particularly, the above mentioned %-identities also apply for the previous SEQ ID NOs. In another, the second nucleic acid molecule comprises at least 4, 5, 10, 15, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1500, 2000, 2500, 3000, 3500, 4000, 4500, 4600, 4700, 4800, 4900, 5000, 5500, 5600, 5700, 5800, 5900, 6000, 6100, 6200, 6300, 6400, 6500 or 6600 neighboured nucleotides of the nucleotide sequence of one of the previously mentioned SEQ ID NOs or a complement thereof.

In a further aspect the invention relates to a method for propagating the IB80 in host cells, comprising the infection of host cells, cell lines and/or egg-systems such as hatching eggs with an isolated infectious bronchitis-virus as described above and according to the invention, the cultivating of the host cells, cell lines and/or egg-systems such as hatching eggs to enable the virus to replicate and the harvesting of resulting virions. Additionally, the invention relates to host cells, cell lines and/or egg-systems such as hatching eggs which are infected with the virus according to the invention and described above. In one embodiment of the present invention, the host cell is a bird cell; herein all bird species are possible according to the invention, particularly chickens. In one embodiment of the present invention, the egg-system particularly relates to hatching eggs. In one embodiment of the present invention, the egg-system particularly relates to embryonised eggs, as example, SPF-eggs, “serum eggs” and clean eggs are to be mentioned here. Egg-systems of all bird species are possible; preferably egg systems of chickens, e.g. chicken eggs. It was found that kidney cells of the vervet monkey (African green monkey) are particularly suitable as host cells. In a further aspect, the invention relates to a method for detecting the presence of an antibody in a biological sample, which specifically binds IB-virus, wherein the method comprises: (a) contacting the biological sample with the host cell according to the invention and as described above; and (b) detecting the antibody bound to the cell.

In a further aspect, the invention relates to vaccine preparations, including the IB-virus according to the invention, including recombinant and chimeric forms of the virus, and the nucleic acid molecules or protein sub-units of the virus comprised by the virus. In an embodiment, the vaccine compositions according to the invention comprise the living but attenuated IB-virus with or without pharmaceutically acceptable carriers, including adjuvants. In another, the vaccine compositions of the invention comprise inactivated or dead IB-virus, variants of the IB-virus or a combination thereof with or without pharmaceutically acceptable carriers including adjuvants. Such attenuated or inactivated viruses can be produced by a series of passages of the virus in a host cell, e.g. a cell line, by inactivation methods of by producing recombinant or chimeric forms of the virus. Thus, the present invention relates to methods for producing the recombinant or chimeric forms of the IB-viruses according to the invention as described herein.

In another specific embodiment, the invention relates to a vaccine formulation comprising a therapeutically or prophylactically effective amount of the IB-virus according to the invention and as described above and a pharmaceutically acceptable carrier. In another, the invention relates to a vaccine formulation comprising a therapeutically or prophylactically effective amount of a protein extract of the IB-virus according to the invention and as described above, or a sub-unit thereof and a pharmaceutically acceptable carrier. In a further aspect, the invention relates to a vaccine formulation comprising a therapeutically or prophylactically effective amount of a nucleic acid molecule, comprising the nucleotide sequence comprising one or more nucleic acid sections selected from the group consisting of nucleic acids of the sequences SEC ID NOs: 1, 2, 3, 4, 5, 6, 7, 8 and 9; or comprising a nucleic acid of SEQ ID NO: 10; or comprising a nucleic acid comprising or consisting of a nucleic acid section with an identity of ≥85% to SEQ ID NO: 3 or with an identity of ≥98% to one of the SEQ ID NOs: 1, 2, 4, 5, 6, 7, 8, 9 or 10; or a complement thereof and a pharmaceutically acceptable carrier. Particularly, the above mentioned %-identities also apply for the previous SEQ ID NOs. In another, the invention relates to a vaccine formulation comprising a therapeutically or prophylactically effective amount of a nucleic acid molecule, comprising any of the nucleotide sequences described above and according to the invention, or a complement thereof and a pharmaceutically acceptable carrier. In a further aspect, the invention relates to a vaccine formulation which comprises a therapeutically or prophylactically effective amount of a protein extract of the IB-virus according to the invention and as described above, or a sub-unit thereof and a pharmaceutically acceptable carrier.

In a further aspect, the invention relates to a vaccine formulation comprising a therapeutically or prophylactically effective amount of a nucleic acid molecule, comprising the nucleotide sequence comprising one or more nucleic acid sections selected from the group consisting of nucleic acids of the sequences SEC ID NOs: 1, 2, 3, 4, 5, 6, 7, 8 and 9; or comprising a nucleic acid of SEQ ID NO: 10; or comprising a nucleic acid comprising or consisting of a nucleic acid section with an identity of ≥85% to SEQ ID NO: 3 or with an identity of ≥98% to one of the SEQ ID NOs: 1, 2, 4, 5, 6, 7, 8, 9 or 10; or a complement thereof and a pharmaceutically acceptable carrier. Particularly, the above mentioned %-identities also apply for the previous SEQ ID NOs. In another, the invention relates to a vaccine formulation comprising a therapeutically or prophylactically effective amount of a nucleic acid molecule, comprising any of the nucleotide sequences according to the invention and as described above, and a pharmaceutically acceptable carrier.

In an even further special embodiment, the vaccine compositions of the present invention comprise a nucleic acid or a fragment of the IB80, e.g. the virus with the deposit number 16061601 or with the deposit number 16070701 or nucleic acid molecules one or more nucleic acid sections selected from the group consisting of nucleic acids of the sequences SEC ID NOs: 1, 2, 3, 4, 5, 6, 7, 8 and 9; or comprising a nucleic acid of SEQ ID NO: 10; or comprising a nucleic acid comprising or consisting of a nucleic acid section with an identity of ≥85% to SEQ ID NO: 3 or with an identity of ≥98% to one of the SEQ ID NOs: 1, 2, 4, 5, 6, 7, 8, 9 or 10; or a fragment thereof. In another, the vaccine preparations comprise a protein/polypeptide of the invention which is encoded by a nucleotide sequence as described previously or a fragment thereof. In a special embodiment, the vaccine preparations comprise proteins/polypeptides of the invention, comprising or consisting of an amino acid chain selected from the group of the amino acid sequences SEQ ID NOs: 11, 12, 13, 14, 15, 16, 17, 18, 19 ad 20; or one or more proteins comprising or consisting of an amino acid chain with an identity of ≥85% to the amino acid sequence SEQ ID NO: 13 or to an amino acid sequence selected from the group of amino acid sequences with an identity to ≥98% to one of the amino acid sequences SEQ ID NOs: 11, 12, 14, 15, 16, 17, 18, 19 or 20; or as they are encoded by one of the nucleotide sequences according to the invention as described above or a fragment thereof.

Further, the present invention relates to methods for treating, alleviating, handling or preventing infectious bronchitis (generally also including other symptoms caused by IB80), by administering vaccine preparations of antibodies of the present invention alone or in combination with adjuvants or other pharmaceutically acceptable adjuvants. Further, the present invention relates to methods for treating, alleviating, handling or preventing infectious bronchitis by administering the compositions and formulations according to the invention, including the vaccine compositions or antibodies of the present invention alone or in combination with antiviral drugs (e.g. Amantadin, Rimantadin, Gancyclovir, Aciclovir, Ribavirin, Penciclovir, Oseltamivir, Foscarnet Zidovudin (AZT), Didanosin (DDL), Lamivudin (3TC), Zalcitabin (ddC), Stavudin (d4T), Nevirapin, Delavirdin, Indinavir, Ritonavir, Vidarabine, Nelfinavir, Saquinavir, Relenza, Tamiflu, Pleconaril, Interferone, etc.) with steroids and corticosteroids such as Prednison, Cortison, Fluticason and glucocorticoid, antibiotics, analgesics, bronchodilators or other treatments for respiratory system and/or viral infections.

In a related aspect, the invention relates to an immunogenic formulation comprising an immunogenically effective amount of the IB-virus according to the invention and as described above and a pharmaceutically acceptable carrier.

In another related aspect, the invention relates to an immunogenic formulation, comprising an immunogenically effective amount of a protein extract described above of the IB80-virus according to the invention or a sub-unit thereof and a pharmaceutically acceptable carrier.

In a further related aspect, the invention relates to an immunogenic formulation comprising an immunogenically effective amount of a nucleic acid molecule comprising one or more nucleic acid sections selected from the group consisting of nucleic acids of the sequences SEC ID NOs: 1, 2, 3, 4, 5, 6, 7, 8 and 9; or comprising a nucleic acid of SEQ ID NO: 10; or comprising a nucleic acid comprising or consisting of a nucleic acid section with an identity of ≥85% to SEQ ID NO: 3 or with an identity of ≥98% to one of the SEQ ID NOs: 1, 2, 4, 5, 6, 7, 8, 9 or 10; or a combination thereof or a complement thereof and a pharmaceutically acceptable carrier. Particularly, the above mentioned %-identities also apply for the previous SEQ ID NOs.

In another related aspect, the invention relates to an immunogenic formulation comprising an immunogenically effective amount of a nucleic acid molecule comprising one of the nucleotide sequences according to the invention and as described above or a complement thereof and a pharmaceutically acceptable carrier.

In another related aspect, the invention relates to an immunogenic formulation comprising an immunogenically effective amount of one of the polypeptides according to the invention and as described above.

In another aspect, the present invention relates to pharmaceutical compositions comprising antiviral agents of the present invention and a pharmaceutically acceptable carrier. In a specific embodiment, the antiviral agent of the invention is an antibody, which immunospecifically binds to IB80 or an IB80-epitope. In another specific embodiment, the antiviral agent is a polypeptide or a protein of the present invention or a nucleic acid molecule of the invention.

In another related aspect the invention relates to a pharmaceutical composition comprising a prophylactically or therapeutically effective amount of an anti-IB-virus agent and a pharmaceutically acceptable carrier. In one embodiment of the present invention, the anti-IB80-agent is an antibody or an antigen binding fragment thereof, which immunospecifically binds to the virus of the deposit number 16061601 or 16070701 or to polypeptides or proteins derived therefrom. In another, the anti-IB80-agent is a nucleic acid molecule comprising one or more nucleic acid sections selected from the group consisting of nucleic acids of the sequences SEC ID NOs: 1, 2, 3, 4, 5, 6, 7, 8 and 9; or comprising a nucleic acid of SEQ ID NO: 10; or comprising a nucleic acid comprising or consisting of a nucleic acid section with an identity of ≥85% to SEQ ID NO: 3 or with an identity of ≥98% to one of the SEQ ID NOs: 1, 2, 4, 5, 6, 7, 8, 9 or 10; or a combination thereof or a fragment thereof. In others, the anti-IB80-agent is a polypeptide, which is encoded by a nucleic acid molecule comprising one of the nucleic acid sequences according to the invention and as described above, a combination thereof or a fragment thereof with a biological activity of the polypeptide. Particularly, the above mentioned %-identities also apply for the previous SEQ ID NOs.

The invention also relates to kits, comprising compositions and formulations of the pre sent invention. Thus in another aspect, the invention relates to a kit comprising a container which contains the immunogenic formulation according to the invention and as de scribed above.

In a further aspect, the invention relates to a kit, including a container, containing the pharmaceutical composition according to the invention as described above.

In a further aspect, the invention relates to a kit including a container which contains the vaccine formulation according to the invention and as described above.

In a further aspect, the invention relates to a method for identifying a subject which is infected with the IB80 according to the invention and as described above, including (a) obtaining the total RNA of a biological sample obtained from the subject; (b) reverse transcription of the total RNA to obtain cDNA; and (c) amplifying the cDNA by using a set of primers which are derived from a nucleotide sequence of the IB-virus according to the invention and as described above.

In an embodiment of the present invention, the set of primers is derived from the nucleotide sequence of the genome of the IB-virus with the deposit number 1606161 or 16070701. In another, the set of primers is derived from one of the nucleotide sequences according to the invention and as described above or a complement thereof.

The invention further relates to the use of the sequence information of the isolated IB80-virus for diagnostic and therapeutic methods. In a special embodiment, the invention relates to nucleic acid molecule which are suitable for use as primers, consisting of or including one of the nucleotide sequence according to the invention as described above or a complement thereof or at least a part of the nucleotide sequence thereof. In another specific embodiment, the invention relates to nucleic acid molecules which are suitable for the hybridisation to the IB80-nucleic acid according to the invention, including but not limited to PCR-primers, reverse transcriptase primers, probes with southern analysis, northern blots, probes for real-time PCR or other hybridisation analyses for nucleic acids for detecting IB80-nucleic acids, e.g. consisting of or including one of the nucleotide sequences according to the invention and as described above or a combination thereof, a complement thereof or a part thereof. The invention further comprises chimeric or recombinant IB80-viruses which are completely or partially encoded by nucleotide sequences.

In another aspect, the present invention relates to methods for screening of antiviral agents which inhibit the infectivity or replication of IB80 including variants thereof.

The invention further relates to methods for producing recombinant or chimeric forms of IB80.

In a further embodiment, the invention relates to vaccine preparations including the IB80-virus, including recombinant and chimeric forms of the IB80-virus or sub-units of the IB80-virus. The present invention comprises recombinant and chimeric viruses, which are encoded by viral vectors, which are derived from the genome of the IB80-virus according to the invention and a s described herein or natural variants thereof. In a special embodiment, the recombinant virus is a virus derived from the IB80 virus with the deposit number 16061601 or 16070701. It is recognized that natural variants of the IB80-viruses according to the invention and as described herein, comprise one or more mutations including, but not restricted thereon, point mutations, rearrangements, insertions, deletions etc. in the genomic sequence. It is recognized that the mutations can or cannot lead to a phenotypic change.

In another specific embodiment, a chimeric virus of the invention is a recombinant IB80-virus which further comprises a heterologous nucleotide sequence. In accordance with the present invention, a chimeric virus, can be encoded by a nucleotide sequence, in which heterologous nucleotide sequences were added to the genome or in which endogenous or native nucleotide sequences were replaced by heterologous nucleotide sequences.

According to the present invention, the chimeric viruses are encoded by the viral vectors of the invention, with further comprise a heterologous nucleotide sequence. In accordance with the present invention, a chimeric virus is encoded by a viral vector which may or may not contain nucleic acids, which are non-native to the viral genome. In accordance with the invention, a chimeric virus is preferably encoded by a viral vector in which heterologous nucleotide sequences are added, inserted or substituted for native or non-native sequences. In accordance with the present invention, the chimeric virus can also be encoded by nucleotide sequences derived from different species or of variants of the IB-virus. Particularly, the chimeric virus is encoded by nucleotide sequences which encode for antigenic polypeptides derived from different species or variants of the IB-virus.

A chimeric virus can be of particular use for the production of recombinant vaccines, for protection against two or more viruses (Tao et al., J. Virol. 72, 2955-2961; Durbin et al., 2000, J. Virol. 74, 6821-6831; Skiadopoulos et al., 1998, J. Virol. 72, 1762-1768 (1998); Teng et al., 2000, J. Virol. 74, 9317-9321). For example, it may be intended that a subject, which is vaccinated with a vector derived from the IB80 virus and which encodes one or more proteins of variants of the IB80-virus, is protected against infections of the native IB80-virus as well as of the variant. Attenuated and replication-deficient viruses may be suitable for vaccinations with live vaccines, as it is proposed for other viruses. (See as e.g. WO 02/057302 on pages 6 and 23; and publication of the US patent application US 2008/0069838, which both are incorporated herein by reference).

In accordance with the present invention, the heterologous sequence to be incorporated into the viral vectors, which encodes for the recombinant or chimeric viruses of the invention, includes sequences which can be obtained or derived from different species or variants of IB80.

In special embodiments, the chimeric or recombinant viruses of the invention are encoded by viral vectors which are derived from viral genomes wherein one or more sequences, intergenetic regions, termini-sequences or parts or complete ORFs are replaced by a heterologous or non-native sequence. In special embodiments of the invention, the chimeric viruses of the invention are encoded by viral vectors which are derived from viral genomes wherein one or more heterologous sequences are inserted or added into the vector.

The selection of the viral vector can depend on the species of the subject which is to be treated or protected regarding a viral infection. An attenuated IB80-virus can be used to provide the antigenic sequences.

In accordance with the present invention, the viral vectors can be constructed to provide antigenic substances which impart protection against an infection of the same by the IB80 according to the invention and natural variants thereof. The viral vectors can be constructed such, to provide on, two, three or more antigenic sequences. In accordance with the present invention, the antigenic sequences can be from the same virus, from different species or variants of the same virus-type of different viruses.

The expression products and/or recombinant or chimeric virions which were obtained according to the invention, can be used in advantageous manner in vaccine formulations. The expression products and chimeric virions of the present invention can be constructed to produce vaccines against a broad spectrum of pathogens including viral and bacterial antigens, tumor antigens, allergen antigens and antigens involved in autoimmune diseases. A possibility to reach this aim includes the modification of existing IB80 such that they contain foreign sequences in their respective external domains. Where the heterologous sequences are epitopes or antigens of pathogens, these chimeric viruses can be used to induce a protective immune response against the pathogen of which the determinants are derived from. Particularly, the chimeric virions of the present invention can be constructed to impart vaccines for the protection of a subject from infections with IB80-virus and variants thereof.

Thus the present invention further relates to the use of viral vectors and recombinant and chimeric viruses according to the invention to formulate vaccines against a broad spectrum of viruses and/or antigens. The present invention also comprises recombinant viruses according to the invention including a viral vector derived from the IB80 or variants thereof, which contains sequences which lead to a virus with a phenotype better suited for the use in vaccine formulations, e.g. an milder phenotype or with improved antigenicity. The mutations and modifications can be in the coding regions, in intergenetic regions and in the leader and trailer sequences of the virus.

The invention relates to a host cell with a nuclei acid or a vector according to the invention. Plasmid or viral vectors containing the polymerase components of the IB80-virus are produced in prokaryotic cells for the expression of the components in the respective cell types (bacteria, insect cells, eukaryotic cells). Plasmid or viral vectors containing the full length or partial copies of the IB80-genome, are produced in prokaryotic cells for the expression of viral nucleic acids in vitro or in vivo. The latter vectors contain where applicable other viral sequences for the production of chimeric viruses or chimeric viral proteins, they lack where necessary parts of the viral genome for the production of a replication deficient virus and the may obtain mutations, deletions or insertions for producing attenuated viruses. Furthermore, the present invention relates to a host cell, cell line and/or egg-system e.g. hatching egg, which is infected with an IB80-virus of the deposit number 16061601 or 16070701. Host cells or, respectively, cell lines are generally accessible via cell banks, egg-systems as e.g. hatching eggs are generally accessible via the respective producers.

Infectious copies of the IBV (as wild type, attenuated, replication deficient or chimeric) are optionally produced during co-expression of the polymerase components according to the technologies in prior art as described above.

Additionally, where applicable, eukaryotic cells, which transiently or stably express one or more IB80-proteins in full-length or partially, are used. Preferably, such cells are produced by transfection (protein or nucleic acid vectors), infection (viral vectors) or transduction (viral vectors) and are suitable for complementation of said wild type, attenuated, replication deficient or chimeric viruses.

The viral vectors and chimeric viruses according to the present invention can where applicable modulate the immune system of a subject, by stimulating a humoral immune response, a cellular immune response, or by stimulating a tolerance against an antigen. As used herein, the term subject describes: preferably humans, primates, horses, cows, sheep, pigs, goats, dogs, cats, birds and rodents. Particularly a subject means: birds, particularly chickens.

### Formulation of Vaccines and Virostatic Agents

In a preferred embodiment, the invention relates to a protein-like molecule or an IB80-virus specific viral protein o a functional fragment thereof which is encoded by a nucleic acid according to the invention. Suitable protein-like molecules originate for example from one of the genes or genomic fragments which are derivable from the virus according to the invention. Such molecules or their antigenic fragments as intended herein, are for example suitable in diagnostic methods or kits and in pharmaceutical compositions as sub-unit-vaccines. Particularly suited are polypeptides which are encoded by a nucleotide sequence according to the invention, comprising one or more nucleic acid sections selected from the group consisting of nucleic acids of the sequences SEC ID NOs: 1, 2, 3, 4, 5, 6, 7, 8 and 9; or comprising a nucleic acid of SEQ ID NO: 10; or comprising a nucleic acid comprising or consisting of a nucleic acid section with an identity of ≥85% to SEQ ID NO: 3 or with an identity of ≥98% to one of the SEQ ID NOs: 1, 2, 4, 5, 6, 7, 8, 9 or 10, or antigenic fragments thereof for the inclusion as antigen or immunogen-subunit, but the inactivated complete virus can also be used. Particularly, the above mentioned % identities also apply for the previous SEQ ID NOs. Particularly suitable are also such protein-like substances, which are encoded by the recombinant nucleic acid fragments of the IB80-genome, preferably are those lying within the preferred limits and ranges of ORFs particularly to evoke specific IBV-antibodies or T-cell-reactions, be I in vivo (e.g. for protecting or therapeutic purposes or for the provision of diagnostic antibodies) or in vitro (e.g. by phage-display-technique or another technique suitable for the production of synthetic antibodies).

It is recognized that numerous variants, analogues or homologues of IB80-polypeptides lie within the scope of the present invention, including substitutions, changes, modifications of amino acids or other changes of amino acids, which increase, reduce or do not change the function or immunogenic tendency of the immunogen according to the invention or the vaccine. Several post-translational modifications are similarly within the scope of the present invention, as including incorporation of naturally occurring amino acid(s), phosphorylation, glycosylation, sulfatylation, and the addition of residues as biotinylation, fluorophores, lumiphores, radioactive residues, antigens or other molecules.

Methods for the expression and purification of natural or recombinant peptides and proteins are well known in the state of the art. For illustration purposes, peptides and proteins are recombinantly expressed in eukaryotic cells. Exemplary eukaryotic cells comprise yeast, HeLa-cells, 293-cells, COS-cells, ovarian-cells of the Chinese hamster (CHO) and many other cell types known in the state of the art. Eukaryotic and prokaryotic expression systems and cells are available, e.g. of Invitrogen Corp., Carlsbad, Calif. Of course, cell free expression systems can be used similarly.

In a preferred embodiment, an immunogenic polypeptide is an IB80-protein in full-length. Preferably, an immunogenic IB80-protein in full-length comprises one or more proteins comprising or consisting of an amino acid chain selected from the group of the amino acid sequences SEQ ID NOs: 11, 12, 13, 14, 15, 16, 17, 18, 19 ad 20; or one or more proteins comprising or consisting of an amino acid chain with an identity of ≥85% to the amino acid sequence SEQ ID NO: 13 or to an amino acid sequence selected from the group of amino acid sequences with an identity to ≥98% to one of the amino acid sequences SEQ ID NOs: 11, 12, 14, 15, 16, 17, 18, 19 or 20 or a fragment thereof, as described herein. Particularly, the above mentioned %-identities also apply for the previous SEQ ID NOs. Preferably, an immunogen has a minimum of 5 amino acids. As used herein, an immungen is preferably a polypeptide. In the context of an immunogenic peptide, the terms immunogen, polypeptide and antigen can be used interchangeably.

Modifications and changes in the structure of the immungens according to the invention can be performed, which are subject-matter of the application and still result in a molecule with similar or improved attributes as the wild type sequence (e.g. a conservative amino acid substitution). For example, where applicable, certain amino acids are substituted by other amino acids in a sequence without significant loss of immunogenic activity. As it is the interactive capacity and nature of a polypeptide, defining the biological functional activity, certain amino acid sequence substitutions can be performed and though a polypeptide with similar or improved attributes can be obtained. Optionally, a polypeptide is used which has less or more immunogenic activity compared to the wild type sequence.

When performing such changes, the hydropathy-index of amino acids is preferably taken into account. The meaning of the hydropathic index during imparting the interactive biological function to a polypeptide, is generally known in the state of the art. It is known that certain amino acids can be replaced by other amino acids with a similar hydropathic index or value and still result in a polypeptide with similar biological activity. A hydropathy-index was allocated to each amino acids based on its hydrophobicity and charge characteristics. These indices are for example according to Kyte and Doolittle: Isoleucine (+4,5); Valine (+4,2); Leucine (+3,8); Phenylalanine (+2,8); Cysteine/Cysteine (+2,5); Methionine (+1,9); Alanine (+1,8); Glycine (−0,4); Threonine (−0,7); Serine (−0,8); Tryptophan (−0,9); Tyrosine (−1,3); Proline (−1,6); Histidine (−3,2); Glutamate (−3,5); Glutamine (−3,5); Aspartate (−3,5); Asparagine (−3,5); Lysine (−3,9); and Arginine (−4,5).

It is assumed that the relative hydropathic character of an amino acid determines the secondary structure of the resulting polypeptide, which in turn defines the interaction of the polypeptide with other molecules such as enzymes, substrates, receptors, antibodies, antigens and the like. It is known in the state of the art that an amino aid can be replaced by another amino acid with similar hydropathy-index and still a functionally equivalent immunogen is obtained. In case of such changes, the substitution of amino acids with an hydropathic index within ±2 is preferred, particularly preferably such within ±1 and especially preferably such within ±0.5.

As described above, amino acid substitutions are generally based on the relative similarity of the amino acid residue substituents such as their hydrophobia, hydrophily, charge, size and the like. Exemplary substitutions taking into account different of the previous attributes and well known to the person skilled in the art comprise (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe) and (Val: Ile, Leu). The embodiments of the disclosure thus consider functional or biological equivalents of a polypeptide and immunogen as described above. Particularly the embodiments of the polypeptides comprise where applicable variants with approximately 50%, 60%, 70%, 80%, 90% and 95% sequence identity to the polypeptide of interest.

The invention relates to vaccine formulations for the prevention and treatment of infections with IB80-virus. In certain embodiments, the vaccine of the invention comprises recombinant and chimeric viruses of the IB80-virus. In certain embodiments, the virus is attenuated.

In another embodiment of this aspect of the invention, inactivated vaccine formulations are produced by using typical techniques to “kill” the viruses. Inactivated viruses are “dead” in such a sense that their infectivity was destroyed and no remaining virus which is able to replicate can be detected. Ideally, the infectivity of the virus is destroyed without influencing its immunogenicity. To produce inactivated vaccines, the virus can be bred in cell culture or in an egg-system e.g. in hatch eggs, particularly in the allantois of a bird embryo (as particularly in the allantois of the chicken embryo), for example purified by zonal ultra-centrifugation, e.g. by inactivation and polarizing with formaldehyde, β-propiolactone, and/or other methods. The resulting vaccine can be inoculated intramuscularly, subcutaneously or intranasaly.

Inactivated viruses are, where applicable, formulated with a suitable adjuvant to increase the immunologic response. Such adjuvants comprise, for illustration but not for limitation, mineral gels such as aluminium hydroxide; surface active substances such as lysolecithine, pluronic-polyols, polyanions; peptides; oil emulsions; and potentially useful animal or human adjuvants such as BCG and Corynebacterium parvum.

In another aspect, the present invention also relates to formulations of DNA-vaccines containing a nucleic acid or a fragment of the IS-virus according to the invention, for example the virus of the deposit number 16061601 or 16070701 or nucleic acid molecules comprising one or more nucleic acid sections selected from the group consisting of nucleic acids of the sequences SEC ID NOs: 1, 2, 3, 4, 5, 6, 7, 8 and 9; or comprising a nucleic acid of SEQ ID NO: 10; or comprising a nucleic acid comprising or consisting of a nucleic acid section with an identity of ≥85% to SEQ ID NO: 3 or with an identity of ≥98% to one of the SEQ ID NOs: 1, 2, 4, 5, 6, 7, 8, 9 or 10; or a fragment thereof. Particularly, the above mentioned %-identities also apply for the previous SEQ ID NOs. In another special embodiment, the DNA-vaccine-formulations according to the present invention comprise a nucleic acid or a fragment thereof which encodes antibodies which immunospecifically bind IS-viruses. DNA-vaccine-formulation comprises a vaccine DNA, a viral vector as it is derived from the IS-virus, a bacterial plasmid or another expression vector which carries an insert with a nucleic acid molecule according to the invention, functionally connected with one or more control elements to enable the expression of the proteins encoded by the nucleic acid molecule in a vaccinated subject. Such vectors can be produced by recombinant DNA-technology as recombinant or chimeric viral vectors which carry a nucleic acid molecule of the present invention.

A nucleic acid as used herein relates to single strand or double strand molecules which may be DNA, including the nucleotide bases A, T, C and G or RNA including the bases A, U (replaces T), C and G. The nucleic acid can represent a coding strand or its complement. Nucleic acids are, where applicable, sequence identical with the sequence occur ring naturally, or comprise alternative codons which encode the same amino acid as is found in the naturally occurring sequence. Further, nucleic acids optionally comprise codons which represent conservative substitutions of amino acids, as they are well known in the state of the art.

As used herein, the term “isolated nucleic acid” describes a nucleic acid which is separated of or substantially free of at least many of the other components of the naturally occur ring organism, for example typically the cell structure components and/or other nucleic acids found associated with nucleic acids in a cellular environment. The isolation of nucleic acids is achieved (for illustration) by techniques such as lysis, followed by phenol and chloroform extraction, followed by ethanol precipitation of the nucleic acids. The nucleic acids of this invention are (for illustration) isolated from cells according to methods which are well known for the isolation of nucleic acids in the state of the art. Alternatively, the nucleic acids of the present invention can, where applicable, be synthesized according to standard protocols for synthesizing nucleic acids described in literature. Modifications of the nucleic acids of the invention are also considered, provided that the essential structure and function of the peptide or polypeptide encoded by the nucleic acid is maintained.

The nucleic acid encoding the peptide or polypeptide according to the invention is, where applicable, part of a recombinant nucleic acid construct, comprising a combination of restriction sites and/or functional elements as they are well known in the state o the art to ease the molecular cloning and other recombinant DNA-manipulations. Thus, the present invention also relates to a recombinant nucleic acid construct containing a nucleic acid which encodes a polypeptide of the invention.

Generally, it can be advantageous to use a cDNA version of the (recombinant) polynucleotide. It is assumed that the use of a cDNA version provides advantages in that the size of the genes can generally be substantially smaller and used more easily to transfect the target cell than a genomic gen which is typically a magnitude bigger than the cDNA gene. However, the invention does not exclude the possibility that where desired a genomic version of a certain gene is used.

As used herein, the terms “constructed” and “recombinant” cells are equal to “host cells” and are intended to relate to a cell or cell line in which an exogenous DNA-segment of gene such as a cDNA or a gene is introduced. Thus, constructed cells are distinguishable from naturally occurring cell which do not contain a recombinantly introduced exogenous DNA-segment or gene. A host cell, or cell line, is where applicable a naturally occurring cell or cell line which is transformed or transfected with an exogenous DNA-segment or gene or a cell or cell line which is not modified. A host cell typically does not have a naturally occurring encoding spike protein gene. Constructed cells or cell lines are thus cells which have a gene or genes introduced by human hand. Recombinant cells comprise (for illustration) such that have an introduced cDNA or genomic DNA or RNA, and also comprise genes which are positioned neighboured to a promotor which is naturally not associated with the certain introduced gene.

To express a recombinantly encoded polypeptide in accordance with the present invention, an expression vector, where applicable, is produced which contains a polynucleotide under control of one or more promotors.

To put an encoding sequence “under control” of a promotor, the 5′-end of the translation initiation site of the reading frame is generally positioned between 1 and 50 nucleotides “downstream” of (i.e. 3′ of) the selected promotor. The promotor “upstream” stimulates the transcription of the inserted DNA and promotes the expression of the encoded recombinant protein. This is the meaning of “recombinant expression” as used herein in this context.

Many standard techniques are available to construct expression vectors containing the respective nucleic acids and transcription/translation control sequences to achieve protein or peptide expression in a plurality of host expression systems. Available cell types for expression comprise, but are not limited to, bacteria such as E. coli and B. subtilis, transformed with recombinant phage-DNA, plasmid-DNA or Cosmid-DNA-expression vectors. Eukaryotic expression systems can also be used such as yeasts, baculoviruses and insect cells or HEK cells.

### The Attenuation of 1680-Virus

The IB80-virus according to the invention or variants thereof are optionally genetically constructed such that the have an attenuated phenotype. Particularly the viruses of the invention show an attenuated phenotype in a subject, to which the virus was administered as vaccine. Attenuation can be achieved by any method known to a skilled person. With out being limited by theory, the attenuated phenotype of the viruses of the invention is achieved, for example by using a virus which is naturally not well replicated in an intended host species, e.g. by reduced replication of the viral genome, by a reduced ability of the virus to infect a host cell or by a reduced ability of the viral proteins to assemble an infectious viral particle relative to the wild type species of the virus.

Attenuation (weakening, reducing) or also reduction of virulence, is understood in microbiology as the targeted reduction of the disease evoking attributes of a pathogen (virulence), wherein simultaneously its replication ability is maintained or only slightly reduced. Within the attenuation, it is also intended to maintain the surface attributes of the pathogen which are substantial for the immune response (epitopes) and thus maintain its immunogenicity. Thus the attenuation is a possibility to produce live vaccines for an active immunisation. The methods used therefore are well known to the person skilled in the art.

During attenuation, the natural attribute of the pathogen of being able to slightly repro duce in a host which is unfavourable for the pathogen, however, not evoking a disease, is used. In viruses it is explained by that those receptors of the viral surface which enable an introduction into a specific target cell are not adapted to the new cells of the new host. Furthermore, the genes for immune evasion and some virulence factors are unnecessary in cell culture and are thus often deleted.

After several passages (e.g. in a cell culture, chicken embryo or in a living animal) those mutants of the pathogen are selected which can still reproduce themselves and are replicated in case of a transfer to the original host (such as the human) in lower amounts or with weaker disease symptoms. For attenuation, the cultivation of the pathogen at more unfavourable, lower temperatures (approximately 25° C.) can be used, at which the pathogens can also lose their virulence.

### The Weakening of the IB80-Virus

The IB80-virus or strains of it according to the invention, are optimally genetically constructed in that way, that they present a weakened phenotype. Especially the viruses of the invention show a weakened phenotype in a subject, which was given the virus as a vaccination. Weakening can be reached by every method, which is known by a person skilled in the art. Without being bound to a theory, the weakened phenotype of this invention is caused by, e.g. by usage of a virus, which is naturally not well replicated in an intended host cell, e.g. by diminished replication of the viral genome, by a reduced ability of the virus to infect a host cell or by a reduced ability of the viral proteins to mount an infectious viral particle relative to the wild type species of the virus.

Under attenuation (weakening, diminishing) or also virulence weakening, attenuation, one understands in the microbiology the targeted diminishing of the pathogenic properties of a pathogen (virulence), wherein though its reproducibility is maintained or only slightly decreased. During the attenuation, it is also aimed to maintain the surface properties of the pathogen (epitopes) and therefore maintain its immunogenicity. Therefore, the attenuation is one possibility to generate live vaccines for an active immunization. The for this purpose used methods are very well known to the skilled person in the art.

During the attenuation, the natural ability of the pathogen is used to also replicate itself slightly in a, for itself unfavorable, host, but often not causing a disease. This is explained in viruses the way, that the receptors on the virus surface, which enable the uptake in a specific target cell, are not adapted to the cells of the new host. Furthermore, the genes for the immune evasion and some virulence factors are unnecessary in cell cultures and are therefore often deleted.

After several passages (e.g. in a cell culture, chicken embryo or a living animal), mutants of the pathogen are selected, which can still replicate or are replicated during the transfer to the original host (such as the human) in small number or with diminished disease symptoms. For the attenuation a breeding of the pathogen at unfavorable, lower temperatures (approximately 25° C.) can/will be used, at which the pathogen also can lose their virulence.

### Production of Antibodies

Technologies for the production of antibodies or as antigenically acting fragments of it, are well known to the person skilled in the art. For example, a monoclonal antibody is targeted against a specific epitope of an antigen. At first, as it was described with the polyclonal antibody production, animals must be immunized and then their plasma cells (from spleen or lymph nodes) are obtained. As the plasma cells have lost the ability for cell division, at first a merge with tumor cells has to be made. The so generated cell hybrids (hybridoma-technology) receive the ability from the plasma cells to produce a certain antibody and to secrete and from the tumor cell the ability to divide itself almost infinitely and therefore to theoretically live infinitely. Due to several separations (cloning) a strain of cells was obtained, which traces back to a single hybridoma-cell and therefore to a single plasma cell. The so obtained cell lines can be now expanded in culture infinitely strongly and therefore also produce theoretically indefinitely large amounts of antibody. As all cells are traced back to a single cell, all cells of a culture are identical copies of on single cell. Because of that, all cells are producing one single, defined antibody, which allows according to his properties (e.g. binding site at the antigen, strength of the binding etc.) a precise definition and is producible on theoretically unlimited amount.

### Pharmaceutical Composition and Kits

The present invention comprises pharmaceutical mixtures (compositions), including antiviral agents of the present invention. In a specific embodiment, the antiviral agent is preferably an antibody, which binds and neutralizes immune specifically the IB80-virus or strains of it or any thereof derived proteins. In another specific embodiment, the antiviral agent is a polypeptide or nucleic acid molecule of the invention. The pharmaceutical compositions are useful as antiviral prophylactic agents, are (for illustration) administered to a subject, if the subject was exposed or is expected to have been exposed to the virus.

There are known a variance of different delivery systems to the person skilled in the art and he understands to manufacture these to prescribe the pharmaceutical mixture (composition) of the invention. Illustrating are named: Encapsulation into liposomes, micro particles, micro capsules, recombinant cells, which express the mutant viruses, and receptor-mediated endocytosis. Methods for the contribution comprise, but are not limited to these, intradermal, intramuscular, intraperitoneal, intravenous, subcutaneous, intranasal, epidural or oral ways. The agents and pharmaceutical mixtures (compositions) of the invention can be prescribed through every suitable way, e.g. trough infusion or bolus injection, trough absorption, through epithelia- or mucus lining (e.g. oral mucosa, cloacal and intestinal mucosa and so on), if needed also together with other biologically active substances. The prescription is systemic or local. In a preferred embodiment it is wishful to introduce the agents and pharmaceutical mixtures (compositions) of the invention in the lung using every suitable way. Pulmonary prescription can also be made, e.g. by usage of an inhalator or nebulizer, and the formulation with an aerosolisation agent.

In the scope of the present invention are preferably present during the mass application of live vaccines at commercial poultry the administration via drinking water or the prescription via spray (aerosol) and are namely IB-viruses, e.g. the viruses according to the invention (IB80), matters. Also suitable is the in the scope of the present invention vaccine application via the eye, the so called “eye-drop method”, wherein a single drop of the vaccination solution, which contains the agent and pharmaceutical mixtures (compositions) of the invention, is dropped into the eye.

### Detection Assays

The present invention concerns also a method for the detection of an antibody which binds immune specifically to the IB80-virus according to the invention, e.g. in a biological sample including blood, serum, plasma, saliva, urate, feces, and so on, of a subject, which is suffering from an IBV-infection. In such a method a sample, e.g. with a IB80-virus or a genomic nucleic acid sequence according to invention immobilized in contact directly on a substrate and the virus-bound antibody is detected directly or indirectly with a suitable method. Detection methods are well known to the person skilled in the art and comprise e.g. hybridization methods, marking methods, detection probes, immunofluorescence assay. It can be in vitro- or in vivo-methods.

### Screening Assay for the Identification of Antiviral Agents

The invention finally concerns a method for the identification of agents and a substance, which inhibits the ability of the IBV virus to infect a host or a host cell. In specific embodiments, the invention concerns methods for the identification of agents or substances, which inhibit the ability of the IBV-virus, especially of the IB80 to replicate in a host or a host cell. Every technology, which is known to a person skilled in the art, can be used herein.

In specific embodiments, the invention concerns methods for the identification of agents or substances, which inhibit the ability of the IB-virus, especially of the IB80, to replicate in a bird species or especially in poultry, e.g. chickens. More precisely, the invention concerns methods for the identification of agents and substances, which inhibit the ability of the IBV-virus, especially the of the IB80, to infect a bird species or especially poultry, e.g. chickens. In specific embodiments, the invention concerns methods for the identification of agents or substances, which inhibit the ability of the IBV-virus, especially of the IB80, to replicate in host ells, cell lines and/or egg systems, e.g. in hatching eggs.

### EXAMPLES

### Example 1: Virus Isolation

The material used fort the virus isolation derived from a monitoring examination of an, at this time clinical inconspicuous, chicken herd. For the cultural cultivation, a pool of caecal tonsils was used. This organ material was added in 15 mL tubes with 1×PBS, pH 7-7.4 with 50 μg/mL Gentamycin and 2.5 μg/ml Amphotericin B in the ratio 1:10. After addition of three stainless steel beads (diameter 6 mm) the sample was homogenized for 30 sec at room temperature in the FastPrep-24 (MP Biomedicals). For clarification of the organ material, it was centrifuged subsequently at 2,000×g and 4° C. for 20 minutes and the supernatant was filtered with a 0.45 μm filter. Subsequently, an incubation of the filtrate at room temperature for 30 minutes follows.

### Example 2: Virus Proliferation

The virus proliferation is made through direct passages of the filtrate of the processed caecal tonsils in SPF chicken eggs (VALO BioMedia). For this purpose, 200 μL each of this material was injected into the Allantois cave (AC) of eggs (hatching day 10) with a cannula (0.5×16 mm) in the sterile working bench (clean room class A). Afterwards, the opening in the egg was closed with Uhu all-purpose glue. The incubation was made for 7 days at 37-38° C. and a relative humidity of approximately 60%.

The inoculated eggs are candled regularly regarding deaths.

After the 7 days, the eggs were cooled at 4° C. for approximately 4 hours. Afterwards, the opening if the eggs was made with a sterile scissor in the area of the air bubble and the harvest of the Allantois liquid was made through a transfer pipette. The harvest of the inoculated eggs was pooled.

Until the further passaging, the harvest was stored at −20° C.

After the unfreezing, the harvest material of the first passage was diluted with 1×PBS, pH 7-7.4 under sterile conditions and again inoculated with 200 μL per egg in the Allantois cave of SPF hatching eggs.

This was closed with Uhu all-purpose glue and incubated for 4 days at 37° C. and a relative humidity of approximately 50% for 4 days and daily candled. A death in the first 24 hours was discarded as unspecific. The remaining eggs was cooled at day 4 for approximately 4 hours at 4° C. and afterwards, the Allantois liquid was harvested as pool under sterile conditions.

After the intermediate storing at −80° C., the Allantois liquid was diluted again with 1×PBS in the ratio of 1:100, as described inoculated into the Allantois cave of SPF-hatching eggs and afterwards their Allantois liquid was harvested under sterile conditions as a pool and frozen in 1-2 mL-aliquots at −80° C.

### Example 3: Virus Detection

The RNA purification from the harvested Allantois liquid of a sample was made with the QIAamp Viral RNA Mini Kit® (Qiagen) following the instructions of the supplier. Afterwards, the IBV status of the samples was checked with a Real-Time PCR with the Kylt® IB virus kit (Kylt) following the instructions of the supplier. The IBV result was positive at a CT-value of 13.86. Following, the samples were examined for the IBV strains 4/91, Arkansas, Massachusetts, D1466, D274, QX, Italy02, Variant 02 and IB80 with the Kylt Real-Time PCRs Kylt® IBVstrain 4/91, Kylt® IBV strain Arkansas, Kylt® IBV strain Massachusetts, Kylt® IBV strain D1466, Kylt® IBV strain D274, Kylt® IBV strain QX, Kylt® IBV strain Italy02, Kylt® IBVstrain 02 and Kylt® IBV strain IB80, following the instructions of the supplier. Only with the Kylt® IBV strain IB80 Kit, a positive result was obtained (CT 18.6), all other IBV strains could not be detected in this sample.

### Example 4: Genome Sequencing

The genome sequencing of the IB virus isolate was made by the Dideoxy method of Sanger (Sanger and Coulsen, 1977) and was conducted with a 2-step RT-PR protocol: The reverse transcription was made with the GoScript™ Reverse Transcription System (Promega) following the instructions of the supplier. Therefore, the reverse transcription was either conducted with the included in the kit random primers (cDNA A) or with 14 IBV specific primers (cDNA B, Franzo et al., 2015, modified, see table 1). For the reverse transcription 20 pmol of the primer mix (˜1.43 pmol per primer) was used on a reaction preparation of 20 μL. The final protocol for the 20 μL preparation is composed as following: 4μ of the RNA purification of the sample, 1 μL primer mix (random-primer or IBV specific primer mix respectively), 4μ GoScript™ 5× reaction buffer, 2.4 μL 25 mM MgCl₂, 1 μL PCR nucleotide mix, 0.5 μL recombinant RNasin® ribonuclease inhibitor, 1 μL GoScript™ reverse transcriptase, 6.1 μL nuclease free water. The temperature profile complies with the instructions of the supplier.

TABLE 1
IBV specific primer for the
reverse transcription

| | | SEQ |
|---|---|---|
| | | ID |
| Primer name | Sequence | No |
| RT-IBVG-1R | TTTAGTAAAAAGACCACC | 21 |
| RT-IBVG-2R | CATACTTTTGCGCATC | 22 |
| RT-IBVG-3R | AGAAAACCTACACCAG | 23 |
| RT-IBVG-4R | GTAAAGAATGTACTAACC | 24 |
| RT-IBVG-5R | ATAGTATCAAAGACTACAGG | 25 |
| RT-IBVG-6R | CTCCATAAGAATCCTG | 26 |
| RT-IBVG-7R | TAAAACTTGGTTGTTCC | 27 |
| RT-IBVG-8R | TTCACATAAAGCATCAAC | 28 |
| RT-IBVG-9R | GTCATACTCAAACTGC | 29 |
| RT-IBVG-10R | CAAAATGCATTACTCGC | 30 |
| RT-IBVG-11R | CATATCTTCTTTTTGACC | 31 |
| RT-IBVG-12R | TTTGAATCATTAAACAGAC | 32 |
| RT-IBVG-13R | ACCAACTTTAGGTGGC | 33 |
| RT-IBVG-14R | TTGCTCTAACTCTATAC | 34 |

For the following amplification of the genome, 14 primer pairs were used (Franzo et al., 2015, modified, see table 2). The 14 amplicons include overlapping sections each to display the whole genome sequence.

TABLE 2
Primer for the genome amplification

| | | Product | SEQ |
|---|---|---|---|
| | | size | ID |
| Primer name | Sequence | (approx.) | No |
| P-IBVG-1F | GCGCTAGATTTCCAACTTAACAAAACG | 1999 bp | 35 |
| P-IBVG-1R | GACTTGCGAAACAAGATGCCAAATGCC | | 36 |
| P-IBVG-2F | TGGAGGCTTGCATATGGAAAAGTGCG | 2333 bp | 37 |
| P-IBVG-2R | GGAATGAAGAGAATTTCTTTATCCTCA | | 38 |
| | ACATCATC | | |
| P-IBVG-3F | CGGAGGATGGTGTTAAATACCGC | 2013 bp | 39 |
| P-IBVG-3R | CAAATAATATTAGAAAGACCAAATA | | 40 |
| | AAGCCAATTCC | | |
| P-IBVG-4F | GATTCTTTTGATGTGTTACGCTAT | 2153 bp | 41 |
| | TGTGCAG | | |
| P-IBVG-4R | CCTGGTTTAGTATACTCACATAC | | 42 |
| | ACTACC | | |
| P-IBVG-5F | CCTAATGGTGTTAGGCTTATAGTTCC | 2162 bp | 43 |
| P-IBVG-5R | GTATCAAAGACTACAGGATCATACC | | 44 |
| | ATTG | | |
| P-IBVG-6F | CAGTTATTATTGGAGTTTGTGCTGAAG | 2077 bp | 45 |
| P-IBVG-6R | GAATCCTGATCCGGAGTTGGACTTGGC | | 46 |
| P-IBVG-7F | GTGGCAGCAGGTAATCAACCTTTAGG | 2150 bp | 47 |
| P-IBVG-7R | ATAAAACTTGGTTGTTCCAATAAC | | 48 |
| | TACAGG | | |
| P-IBVG-8F | GTGTCTATCCTTTCTACTATGACT | 2074 bp | 49 |
| | AATAGGC | | |
| P-IBVG-8R | CACATAAAGCATCAACAGCTGCATGAG | | 50 |
| P-IBVG-9F | GGCAAGCAGAAGCGTACTACAGTAC | 2064 bp | 51 |
| P-IBVG-9R | CTGCTTGACATTGGGTACTATT | | 52 |
| | GGATTC | | |
| P-IBVG-10F | CGTTGTCTATGATATAGGCAACCC | 1756 bp | 53 |
| | TAAAGG | | |
| P-IBVG-10R | GTATTGACAGAGTTGTGTATACTT | | 54 |
| | TGCC | | |
| P-IBVG-11F | GTAACAGTGTCAATTGATTACCAT | 2757 bp | 55 |
| | AGC | | |
| P-IBVG-11.2R | TCCATACGCGTTTGTATGTACTCA | | 56 |
| | TCTG | | |
| P-IBVG-12.2F | CATAGGCGTAGAAGGTCTATTAGTG | 2011 bp | 57 |
| P-IBVG-12.2R | TTATTTGCTGGAGTGCTATAACAC | | 58 |
| | ACTC | | |
| P-IBVG-13F | CATTATGCCTCTAATGAGTAAGTG | 2245 bp | 59 |
| | TGG | | |
| P-IBVG-13R | AACTTTAGGTGGCTTTGGTCCTCC | | 60 |
| P-IBVG-14F | GAAAAGCGCGAATTTATCTGAGAG | 1845 bp | 61 |
| | AAGG | | |
| P-IBVG-14R | CATAGCCAATTAAACTTAACTTAAA | | 62 |
| | CTAAAATTTAGCTC | | |

The amplification was made with the KOD Hot Start DNA Polymerase® Kit (Novagen) following the instructions of the supplier. The amplification with the single primer pairs (1-14) was made each with the cDNA A, which was synthesized with the random primers, and also with the CDNA B, which was generated with the IBV specific primers. The protocol for the 20 μL preparation is the following: 1 μL cDNA, 2μ 10× buffer for KOD Hot Start DNA Polymerase®, 1.2 μL 25 mM MgSO₄, 2 μL dNTPs (2 mM each), 0.8 μL For ward Primer (10 μM), 0.8 μL reverse primer (10 μM), 0.4 μL KOD Hot Start DNA Polymerase®, 0.8 μL reverse primer (10 μM), 0.4 μL KOD Hot Start DNA Polymerase® (1 U/μL), 10.8 μL nuclease-free water. After the initial denaturation for 2 minutes at 95° C., 40 cycles at 95° C. for 20 sec, 60° C. for 10 sec and 70° C. for 40 seconds each were made (Deviations for defined primer pairs can be seen in table 3)

TABLE 3
Deviations regarding the PCR amplification

| PCR product | Deviations from the standard protocol |
|---|---|
| 1 | None |
| 2 | Extension 50 sec |
| 3 | None |
| 4 | Extension 50 sec, Annealing 56° C. |
| 5 | None |
| 6 | None |
| 7 | None |
| 8 | None |
| 9 | Extension 50 sec, Annealing 56° C. |
| 10 | None |
| 11 | Extension 50 sec |
| 12 | Extension 45 sec |
| 13 | Extension 50 sec, Annealing 58° C. |
| 14 | None |

The success of the amplification was checked afterwards with capillary electrophoresis with the QIAxcel System® (Qiagen) and the QIAxcel DNA Screening Kit. Depending on the quality of the bands, either the product, which was generated on the basis of the cDNA and the random primer, selected or that, which was generated on the basis on the cDNA with the IBV-specific primers (cDNA B) (see table 4).

TABLE 4
Selection of PCR products for the sequencing

| PCR product | cDNA |
|---|---|
| 1 | B |
| 2 | A |
| 3 | B |
| 4 | B |
| 5 | A |
| 6 | B |
| 7 | A |
| 8 | B |
| 9 | B |
| 10 | A |
| 11 | A |
| 12 | A |
| 13 | B |
| 14 | A |

The purification of the PCR product was made with the QIAquick PCR Purification Kit (Qiagen) following the instructions of the supplier.

The DNA concentration of the purified PCR products was determined with the NanoDrop® measurement principle and a NanoDrop® 2000c spectrophotometer. The sequencing reaction was made by MWG Eurofins (Ebersbach, Germany) with the Mix2Seq Kit following the instructions of the supplier. Each PCR product was sequenced with the PCR primers (forward and reverse) and also in general with an additional primer, which lays more in the middle of the product. For the PCR product 10, no additional internal primer was needed. For the PCR products 2 and 11, two internal primers were used for the sequencing (see table 5).

TABLE 5
Sequences, which lay in the
middle of the product

| | | SEQ |
|---|---|---|
| | | ID |
| Primer name | Sequence | No |
| S-IBVG-1F | CCTAAGGATTACGCTGAAGCCTTTGC | 63 |
| S-IBVG-2F | CACACCAATGTCTCAACTTGGTGC | 64 |
| S-IBVG-2R | TCCAATGCTTTGGCAATCACTACCGT | 65 |
| S-IBVG-3.2F | GTAGAAGCCTCACTACCATATCTGT | 66 |
| S-IBVG-4F | GTTAAACCTACAGCATATGCTTACC | 67 |
| S-IBVG-5F | GTATGATGGCAACGAGTTTGTTGG | 68 |
| S-IBVG-6F | TCCTTGCATCTGATGATGTTGGAGAG | 69 |
| S-IBVG-7F | TGACCCAAAGGATTGTGAAGATCTC | 70 |
| S-IBVG-8F | CAAGGTCTTGTAGCAGATATTTCTGG | 71 |
| S-IBVG-9F | CATGAAAGTGGTTCAGCCTACAAC | 72 |
| S-IBVG-11F | TGCAAGTGCAAAGGTTAAAGTTAGTG | 73 |
| S-IBVG-11R | GTAAGCATAACAGCAAGTATGAGATG | 74 |
| S-IBVG-12.2F | CTAGTTCATTAGTAGCCTCAATGGCT | 75 |
| S-IBVG-13F | CTTGGTACTGAACAAGCAGTTCAGC | 76 |
| S-IBVG-14F | TCGTGCAGCAAAGATTATTCGGGAC | 77 |

The evaluation of the sequencing was made with the software DNASTAR of the company Lasergene. Herein, the single chromatograms were checked before and cut to size. The single fragments were afterwards aligned to a reference sequence to assemble the whole genome sequence.

Literature to the examples mentioned above:
Giovanni Franzo, Valeria Listorti, Clive J. Naylor, Caterina Lupini, Andrea Laconi, Viviana Felice, Michele Drigo, Elena Catelli, Mattia Cecchinato, 2015. Molecular investigation of a full-length genome of a Q1-like IBV strain isolated in Italy in 2013. Virus Research 210, 77-80

### Example 5: Test System for the Immune Specificity

### Method:

1. Coating of the plates (e.g. 96-well immune-plates Maxisorb® Nunc, Wiesbaden) with antigen: antigens (could be e.g. Allantois liquid, egg skin, embryo homogenized, cell culture material each loaded with IB80 (or preferably IB80 particles)) mixed 1:4 (vol/vol) with coating buffer or undiluted if the tissue homogenization was made in coating buffer. Pipette 100 μL in each well; cover with adhesive film (also in all following steps); hold plates at 30° C. for 2 hours.
2. Washing: Pouring off. Wash plates 2× with wash buffer. Tap plates dry on top of a staple of towels.
3. Blocking: Pipette 200 μL of blocking solution in each well; Hold plates at 30° C. for 2 hours.
4. Washing: Pour off blocking solution, Wash plates 3× carefully, tap dry shortly.
5. Addition of the to be tested antibody in suitable solution, in doubt in PBS, pH 7.4
6. Binding. Plates 2 hours at room temperature.
7. Washing. Wash plates 3× with washing buffer; tap dry shortly.
8. Detection of the antibody-antigen binding with a suitable detection system, preferred with a secondary antibody
9. Evaluation: The evaluation is made against a testing row, which was treated the same way as the antigen loaded testing row, in which however an antigen is not contained (control). If a high antibody binding is observed with the antigen loaded samples, immune specificity of the tested antibody is present.

### Used Solutions:

PBS, pH 7,4
One liter of solution contains:
8.0 g Sodium chloride (NaCl)
5 0.2 g Potassium chloride (KCl)
1.42 g Disodiumhydrogenphosphate (Na2HPO4)
OR 1.78 g Disodiumhydrogenphosphate Dihydrate (Na2HPO4·2H2O)
0.27 g Potassiumhydrogenphosphat (KH2PO4)
Wash buffer
Tween 20 0.05% (Sigma Aldrich, Munich)
in PBS
Coating buffer
Na2CO3 1.59 g/L
NaHCO3 2.93 g/L
15 NaN3 0.2 g/L
pH 9.6
Blocking Solution
PBS with 2% Skim-milk powder or 0.1% BSA (bovine serum albumin)

1. An isolated Infectious Bronchitis (IB)-virus according to one of the deposit numbers 16061601 and 16070701 at the National Collection of Pathogenic Viruses (NCPV), UK, or comprising
a) one or more nucleic acid sections selected from the group consisting of nucleic acids having a nucleotide sequence as set forth in any of SEQ ID NOs: 1, 2, 3, 4, 5, 6, 7, 8 and 9,
b) a nucleic acid having the nucleotide sequence set forth as SEQ ID NO: 10,
c) one or more proteins, comprising or consisting of an amino acid sequence selected from the group consisting of the amino acid sequences set forth as SEQ ID NOs: 11, 12, 13, 14, 15, 16, 17, 18, 19 and 20,
d) a nucleic acid, comprising or consisting of a nucleotide sequence with an identity of ≥85% to the nucleotide sequence set forth as SEQ ID NO: 3 or with an identity of ≥98% to one of the nucleotide sequences set forth as SEQ ID NOs: 1, 2, 4, 5, 6, 7, 8, 9 or 10
and/or
e) one or more proteins, comprising or consisting of an amino acid sequence with an identity of ≥85% to the amino acid sequence set forth as SEQ ID NO: 13 or comprising or consisting of an amino acid sequence selected from the group consisting of amino acid sequences with an identity of ≥98% to one of the amino acid sequences set forth as SEQ ID NOs: 11, 12, 14, 15, 16, 17, 18, 19 or 20.

2. An isolated nucleic acid as defined in claim 1.

3. An isolated protein as defined in claim 1 and/or coded by a nucleic acid as defined in claim 1.

4. A vector, comprising a nucleic acid section as defined in claim 1.

5. An isolated antibody or isolated antigen binding fragment thereof, binding immunospecifically to a virus, a protein or a nucleic acid, each as defined in claim 1.

6. An isolated IB-virus according to claim 1 which is dead or attenuated.

7. A vaccine, comprising a therapeutically and/or prophylactically active amount of an IB-virus according to claim 1 and a pharmaceutically acceptable carrier.

8. A method for preventing symptoms or diseases caused by a virus according to claim 1 in a subject, comprising administering to the subject a vaccine comprising a therapeutically and/or prophylactically active amount of an IB-virus according to claim 1.

9. Use of an IB-virus according to claim 1 for the production of monoclonal or polyclonal antibodies.

10. A pharmaceutical composition, comprising a therapeutically and/or prophylactically active amount of an antibody or of an isolated fragment thereof binding to an antigen for the therapeutic or prophylactic treatment of symptoms or diseases caused by a virus according to claim 1.

11. A vaccine, comprising a therapeutically and/or prophylactically active amount of an IB-virus according to claim 6 and a pharmaceutically acceptable carrier.

12. A vaccine, comprising a therapeutically and/or prophylactically active amount of one or more nucleic acids according to claim 2 and a pharmaceutically acceptable carrier.

13. A vaccine, comprising one or more proteins according to claim 3 and a pharmaceutically acceptable carrier.

14. A method for preventing symptoms or diseases caused by a virus according to claim 1 in a subject, comprising administering to the subject a vaccine comprising a therapeutically and/or prophylactically active amount of an IB-virus according to claim 6.

15. A method for preventing symptoms or diseases caused by a virus according to claim 1 in a subject, comprising administering to the subject a vaccine comprising a therapeutically and/or prophylactically active amount of one or more nucleic acids according to claim 2.

16. A method for preventing symptoms or diseases caused by a virus according to claim 1 in a subject, comprising administering to the subject a vaccine comprising a therapeutically and/or prophylactically active amount of one or more proteins according to claim 3.

17. Use of an IB-virus according to claim 6 for the production of monoclonal or polyclonal antibodies.

18. Use of one or more nucleic acids according to claim 2 for the production of monoclonal or polyclonal antibodies.

19. Use of one or more proteins according to claim 3 for the production of monoclonal or polyclonal antibodies.
